(19) United States
(12) Patent Application Publication
(10) Pub. No.: US 2025/0340912 A1
(43) Pub. Date: Nov. 6, 2025

(54) METHOD FOR PRODUCING 2,4-DIHYDROXY BUTYRATE OR L-THREONINE USING A MICROBIAL METABOLIC PATHWAY

(71) Applicant: TECHNISCHE UNIVERSITÄT DRESDEN, 01069 Dresden (DE)

(72) Inventors: THOMAS WALTHER, Dresden (DE); CLAUDIO FRAZAO, Dresden (DE)

(73) Assignee: TECHNISCHE UNIVERSITÄT DRESDEN, 01069 Dresden (DE)

(21) Appl. No.: 18/272,987

(22) PCT Filed: Jan. 18, 2022
(86) PCT No.: PCT/DE2022/100042

§ 371 Date: Jul. 18, 2023

(30) Foreign Application Priority Data
Jan. 19, 2021 (DE) 10 2021 101 004.7

(51) Int. Cl. C12P 13/08; C12N 1/20; C12N 9/04; C12N 9/10; C12N 9/12; C12N 9/18; C12N 9/88; C12P 7/42; C12R 1/19

(52) U.S. Cl. CPC C12P 13/08; C12N 1/20; C12N 9/0006; C12N 9/1096; C12N 9/1205; C12N 9/18; C12N 9/88; C12P 7/42; C12R 2001/19; C12Y 101/0103; C12Y 101/01122; C12Y 206/01021; C12Y 207/01039; C12Y 301/01025; C12Y 401/02004; C12Y 402/03001; C12Y 403/01019

(57) ABSTRACT

A method for producing 2,4-dihydroxybutyrate (DHB) or L-threonine using a microbial metabolic pathway is disclosed, by expressing the metabolic pathway in a microbial production strain which was previously modified with respect to its natural wild type form by introducing at least one of the genes necessary for the expression of those enzymes used for the enzymatic conversions into the production strain.

The invention relates to a method for producing 2,4-dihydroxy butyrate (DHB), which may be present in the form of a 2,4-dihydroxy butyrate salt or in the form of the acid 2,4-dihydroxy butyric acid, or L-threonine using a microbial metabolic pathway.

There is a central bioeconomic and ecological interest in reducing the dependence on fossil raw materials by enabling a biologically sustainable production of fuels and chemicals on a biological basis. In this context, there is also a huge increase in the industry's efforts in the biosynthesis of (L)-2,4-dihydroxybutyrate (DHB) due to its importance as a starting material for the synthesis of methionine analogues for animal nutrition.

The amino acid methionine and the methionine analogue (D/L)-2-hydroxy-4-(methylthio)butyrate (HMTB) are mainly used as a feed additive in chicken breeding and generate an annual turnover of approximately 3 billion euros on the market. Methionine is currently produced exclusively from the fossil raw materials petroleum and natural gas. The amino acid threonine is employed as a feed additive in pig fattening. Manufacturers of methionine have a strong interest in converting their chemical methionine production processes to sustainable microbial production processes, since a strong increase in the costs of these processes is to be expected with the increasing price of CO2 emissions from chemical processes.

The amino acid L-threonine is currently prepared on an industrial scale by microbial production processes from the sugars glucose or sucrose. The amino acid D/L-methionine and the analogue D/L-2-hydroxy-4(methylthio)butyrate (HMTB), which can be used equivalently, are currently produced exclusively from petroleum and natural gas on an industrial scale.

The chemical synthesis of methionine from petroleum and natural gas is not sustainable and must be replaced by processes which use renewable raw materials. The microbial synthesis of threonine and methionine from sugars is significantly more sustainable. However, these processes compete with food production.

The object the invention is based on is to provide a method which opens the way for a sustainable method for the preparation of the amino acids methionine and threonine.

The object of the invention is achieved with a method having the features of claim 1. Further developments are stated in the dependent claims.

The solution is a method for producing 2,4-dihydroxy butyrate or L-threonine using a microbial metabolic pathway comprising at least the following steps:
a step of enzymatic conversion of glycolaldehyde to threose using a threose-aldolase,
a step of enzymatic conversion of threose to threono-1,4-lactone using a threose dehydrogenase,
a step of enzymatic conversion of threono-1,4-lactone to threonate using a threono-1,4-lactonase and
a step of enzymatic conversion of threonate to 2-keto-4-hydroxybutyrate (OHB) using a threonate-dehydratase,
wherein the metabolic pathway further comprises a step of enzymatically converting OHB to 2,4-dihydroxybutyrate using an OHB reductase or a step of enzymatically converting OHB to L-homoserine using an L-homoserine transaminase, followed by a step of enzymatically converting L-homoserine to O-phospho-L-homoserine using a homoserine kinase under ATP consumption and a step of enzymatically converting O-phospho-homoserine to L-threonine using an L-threonine synthase, and wherein the metabolic pathway is expressed in a microbial production strain which is modified beforehand with respect to its natural form (wild type) by introducing at least one of the genes necessary for the expression of said enzymes into the production strain in a suitable manner. In accordance with the concept of the invention, the metabolic pathway proceeds via glycol aldehyde, which is obtainable in different ways. Thus, glycolaldehyde can be provided via a synthetic metabolic pathway from xylose, as is described in Cam et al./2016/ACS Synth Biol5/607-618. Furthermore, glycolaldehyde can also be represented from ethylene glycol. This can in turn be easily recovered from synthesis gas, which is just as state of the art as the recovery of synthesis gas from CO2 emissions. In addition, ethylene glycol is also obtainable via the chemical hydrogenolysis of sugars, as described by Zheng et al./2017/ACS Catal/7/1939-1954. Hydrogenolysis of sugars to ethylene glycol is an innovative method for the chemical digestion of plant waste, which can potentially be more efficient than conventional acid- or enzyme-based digestion methods. Last but not least, ethylene glycol is a main component of the plastic polyethyleneterephthalate (PET), so that the method according to the invention also opens upa path for the recycling of plastic waste.

According to the concept of the invention, a metabolic pathway was developed which enables the carbon-preserving conversion of glycolaldehyde, which is in turn readily obtainable from ethylene glycol, to L-threonine or HMTB.

A further substantial advantage is that only few by-products are formed in the ethylene glycol-based production. This is to be expected due to the better separation of the metabolic pathway used for production from the natural metabolism. Due to the presence of few by-products in ethylene glycol-based processes, the purification of the resulting valuable substances threonine and DHB can be created in a relatively simple manner.

In the invention, a metabolic pathway can be realized which does not exist in nature in this form. This metabolic pathway is partly based on enzymatic activities which were not known up to now. Surprisingly, these two unknown enzyme activities could be found through screenings. In addition, the newly found enzyme activities together with already known activities from various other microorganisms could be expressed together in a single production strain. Thus, a previously unknown reaction sequence or a previously unknown metabolic pathway was constructed. According to an advantageous embodiment of the invention, the production strain already has one or more enzymes required for the metabolic pathway in its natural form. Advantageously, a strain of the species Escherichia coli can be used as a production strain, preferably E. coli ΔyqhD ΔaldA. This strain is advantageously suitable as a production strain because it has an inactivation of the enzymes aldehyde dehydrogenase (AldA) and glycol aldehyde reductase (YqhD) which competes with the conversion of glycol aldehyde to D-threose. Furthermore, a strain of the species Pseudomonas putidais also suitable, especially since strains of this species have a very suitable ethylene glycol dehydrogenase.

In one embodiment of the invention, glycolaldehyde is converted to D-threose using a D-threose aldolase. Accordingly, D-threose is then enzymatically converted to D-threono-1,4-lactone using a D-threose dehydrogenase. This is followed by a step of enzymatic conversion of D-threono-1,4-lactone to D-threonate using a D-threono-1,4-lactonase. In the next step in this embodiment there is the enzymatic conversion of D-threonate to 2-keto-4-hydroxybutyrate (OHB) using a D-threonate-dehydratase.

In a particularly preferred embodiment of the method according to the invention, the genetic information expressing the enzyme D-threo-aldose-1-dehydrogenase from Paraburkholderia caryophylli (Pc.TadH) and/or from Xanthomonas campestris (Xc.Fdh) is introduced into the genome of the production strain for the expression of the D-threose dehydrogenase in the production strain. Similarly, for the provision of the D-threose dehydrogenase in the production strain, a genetic information expressing the enzyme D-arabinose dehydrogenase from Saccharomyces cerevisiae (Sc. Ara1) or from Acidovorax avenae (Aa. TadH) or a genetic information expressing the enzyme L-fucose dehydrogenase from Burkholderia Multivorans (Bm. Fdh) can be introduced into the genome of the production strain. Thus, the D-threose dehydrogenase can be represented by one of the amino acid sequences SEQ ID No. 113, SEQ ID No. 117, SEQ ID No. 123, SEQ ID No. 125 and SEQ ID No. 131.

The expression of the D-threonate dehydratase in the production strain can advantageously be realized in that the genetic information expressing the enzyme D-arabinonate dehydratase from Acidovorax avenae (Aa.AraD) and/or Herbaspirillum huttiense (Hh.AraD) and/or Paraburkholderia mimosarum (Pm.AraD) and/or from the optimized mutant Hh.AraD C434S in introduced into the genome of the production strain. The following amino acid sequences can thus represent the D-threonate dehydratase: SEQ ID No. 151, SEQ ID No. 153, SEQ ID No. 155 and SEQ ID No. 159.

For the expression of the D-threose aldolase in the production strain, preferably the genetic information expressing the enzyme D-fructose-6-phosphate aldolase from Escherichia coli (Ec.FsaA) and/or the genetic information of the mutated variant Ec.FsaA L107Y:A129G (Ec.FsaA^{TA}) is introduced into the genome of the production strain. Thus, the D-threose aldolase can be represented by one of the amino acid sequences SEQ ID NO. 109 or SEQ ID No. 111.

For the expression of the threono-1,4-lactonase in the production strain, the genetic information expressing the enzyme gluconolactonase from Thermogutta terrifontis (Tt.Lac11) and/or, particularly preferably, the genetic information of a truncated variant of this enzyme (Tt.Lac11 v1), which leads to a considerable improvement in the expression, can be introduced into the genome of the production strain. Therefore, the threono-1,4-lactonase can be represented by one of the amino acid sequences SEQ ID No. 133 and SEQ ID No. I35.

According to a particularly advantageous design of the method according to the invention, in addition to the enzymes of the respective metabolic pathway, a threonate-importing enzyme is expressed in the production strain. This can be realized, for example, by introducing the genetic information expressing D-threonate-importing permease from Cupriavidus necator (Re.kdgT) into the genome of the production strain. Therefore, the threonate-importing enzyme can be represented, for example, by the amino acid sequence SEQ ID No. 165.

According to the invention, an OHB reductase is used for the conversion of OHB to DHB. In one embodiment variant of the invention, the NADH-dependent OHB reductase Ec.Mdh^{5Q} which is known from Frazão, C. J. R.; Topham, C. M.; Malbert, Y.; François, J. M.; Walther, T. Rational Engineering of a Malate Dehydrogenase for Microbial Production of 2,4-Dihydroxybutyric Acid via Homoserine Pathway. Biochem. J. 2018, 475 (23), 3887-3901 is used as OHB reductase. The OHB reductase can be represented by the amino acid sequence SEQ ID No. 163.

Surprisingly, the reduction of OHB to DHB can be improved by using the co-factor NADPH instead of NADH in the reduction of OHB to DHB. By introducing mutations into the NADH-dependent Ec.Mdh^{5Q} enzyme, it is possible to change its co-factor specificity in favor of the NADPH. This is remarkable in that no NADPH-dependent OHB reductases have existed so far.

In an advantageous embodiment of the invention, a NADPH-dependent variant of the Ec.Mdh^{5Q} enzyme is expressed in the production strain in the biosynthesis of DHB, which variant has a mutation in at least one of the positions D34 or I35. That is, for the expression of the NADPH-preferring OHB reductase in the production strain, the genetic information expressing a mutated variant of the enzyme L-malate dehydrogenase from Escherichia coli (Ec.Mdh) is introduced into the genome of the production strain, wherein the mutated enzyme, in addition to five point mutations of the variant Ec.Mdh^{5Q} (Ec.Mdhl12V:R81A:M85Q:D86S:G179D) mutated relative to the wild type enzyme Ec.Mdh, in which at position 12 isoleucine is replaced by valine (I12V), at position81 arginine is replaced by alanine (R81A), at position 85 methionine is replaced by glutamine (M85Q), at position 86 aspartic acid is replaced by serine (D86S) and at position 179 glycine is replaced by aspartic acid (G179D), has another mutation in at least one of positions D34 and I35. In doing so, D34 denotes the position corresponding to position 34 in the wild-type enzyme which is occupied by aspartic acid, and I35 denotes the position corresponding to the position in the wild-type enzyme which is occupied by isoleucine. Preferably, for the expression of the NADPH-preferring OHB reductase in the production strain, the genetic information expressing one of the following enzymes is introduced into the genome of the production strain: Ec.Mdh I12V:R81A:M85Q:D86S:G179D:D34G (Ec.Mdh^{5Q} D34G), in which aspartic acid in position 34 is replaced by glycine, represented by the amino acid sequence SEQ ID No. 173, Ec.Mdh I12V:R81A:M85Q:D86S:G179D:I35S (Ec.Mdh^{5Q I}35S), in which isoleucine in position 35 is replaced by serine, represented by the amino acid sequence SEQ ID No. 175, Ec.Mdh I12V:R81A:M85Q:D86S:G179D:D34G:I35K (Ec.Mdh^{5Q} D34G I35K), in which aspartic acid in position 34 is replaced by glycine and isoleucine in position 35 is replaced by lysine, represented by the amino acid sequence SEQ ID No. 177, Ec.Mdh I12V:R81A:M85Q:D86S:G179D:D34G:I35R (Ec.Mdh^{5Q} D34G I35R=Ec.Mdh^{7Q}), in which aspartic acid in position 34 is replaced by glycine and isoleucine in position 35 is replaced by arginine, represented by the amino acid sequence SEQ ID No. 179, Ec.Mdh I12V:R81A:M85Q:D86S:G179D:D34G:13 5S (Ec.Mdh^{5Q} D34G I35S), in which aspartic acid in position 34 is replaced by glycine and isoleucine in position 35 is replaced by serine, represented by the amino acid sequence SEQ ID No. 181, and Ec.Mdh I12V:R81A:M85Q:D86S:G179D:D34G:I35T (Ec.Mdh^{5Q} D34G I35T), in which aspartic acid in position 34 is replaced by glycine and isoleucine in position 35 is replaced by threonine, represented by the amino acid sequence SEQ ID No. 183. In a particularly advantageous embodiment of the invention, the enzyme Ec.Mdh I12V:R81A:M85Q:D86S:G179D:D34G:I35R (Ec.Mdh^{7Q}) is expressed as NADPH-dependent OHB reductase.

A corresponding NADPH-dependent variant of the Ec.Mdh^{5Q} enzyme with 2-keto-4-hydroxybutyrate (OHB) reductase activity, which catalyzes the conversion of 2-keto-4-hydroxybutyrate (OHB) to 2,4-dihydroxybutyrate (DHB) and represents a mutant of the L-malate dehydrogenase from Escherichia coli (Ec.Mdh) and, in addition to the five point mutations I12V, R81A, M85Q, D86S and G179D in at least one of positions D34 and I35 has a further mutation compared to the wild type enzyme, also represents an independent object within the invention with the mentioned embodiments. The same also applies to the use of such an enzyme for the conversion of OHB into 2,4-DHB, wherein the use of the enzyme is also included in methods which use metabolic pathways which produce DHB other than those already mentioned above, but which, like these, also comprise an enzymatic conversion of 2-keto-4-hydroxybutyrate (OHB) to 2,4-dihydroxybutyrate (DHB) using an OHB reductase, such as, for example, the method according to WO 2014/009435 A1. As a rule, a correspondingly modified microorganism expresses the genes required for the catalysis of the steps of the selected DHB-producing metabolic pathway as a production strain for the preparation of 2,4-dihydroxybutyrate (DHB). Since each one of these metabolic pathways comprises the enzymatic conversion of 2-keto-4-hydroxybutyrate (OHB) to 2,4-dihydroxybutyrate (DHB) using an OHB reductase, the genetic information expressing one of the above-mentioned enzymes can be introduced into the genome of the microorganism for the expression of the OHB reductase.

Alternatively, starting from the glycolaldehyde, a conversion of the glycolaldehyde to L-threose instead of D-threose can also take place. For this purpose, activities of aldolases selected from enzymes of the known enzyme classes D-threonine aldolase (enzyme class 4.1.2.42), L-allo-threonine aldolase (4.1.2.49), L-threonine aldolase (4.1.2.5), 4-hydroxy-2-oxoglutarate aldolase (4.1.3.16) and 2-dehydro-3-deoxy-D-pentonate aldolase (4.1.2.28) can be used. All further stages of the above-described metabolic pathway up to the 2-keto-4-hydroxybutyrate are possible analogously with the L-form. So, Kim, Suk Min, Hyun Seung Lim, and Sun Bok Lee. “Discovery of a RuBisCO-like Protein that Functions as an Oxygenase in the Novel D-Hamamelose Pathway.” Biotechnology and bioprocess engineering 23.5 (2018): 490-499 could already demonstrate L-threose dehydrogenase activity with hamamelose dehydrogenase from Ochrobactrum anthropi (Oa.HamH). A lactonase enzyme with activity on L-threono-1,4-lactone is described, for example, in Westlake, A. “Thermostable Enzymes Important For Industrial Biotechnology.” (2019). There, gluconolactonase from Thermogutta terrifontis (Tt.Ara11) showed a corresponding activity. Dehydratase enzymes with activity on L-threonate are known, for example dihydroxy acid dehydratase from Sulfolobus solfataricus, as is described in the publication Kim, S.; Lee, S. B. Catalytic Promiscuity in Dihydroxy-Acid Dehydratase from the Thermoacidophilic Archaeon Sulfolobus solfataricus. J. Biochem. 2006, 139 (3), 591-596.

According to a further design of the invention, the above-mentioned methods comprise at least one further, preceding step for the microbial production of glycolaldehyde, for example from ethylene glycol, methanol or xylose. In doing so, glycolaldehyde can be derived from ethylene glycol via a metabolic pathway which, for the conversion of ethylene glycol, either uses the enzyme activities of the pyrroloquinoline quinone (PQQ)-dependent ethylene glycol dehydrogenase (membrane-bound), reported by Mückschel, B.; Simon, O.; Klebensberger, J.; Graf, N.; Rosche, B.; Altenbuchner, J.; Pfannstiel, J.; Huber, A.; Hauer, B. Ethylene Glycol Metabolism by Pseudomonas Putida. Appl. Environ. Microbiol. 2012, 78 (24), 8531-8539, or the NAD(P)-dependent ethylene glycol dehydrogenase (cytosolic), known from Lu, Z.; Cabiscol, E.; Obradors, N.; Tamarit, J.; Ros, J.; Aguilar, J.; Lin, E. C. Evolution of an Escherichia Coli Protein with Increased Resistance to Oxidative Stress. J. Biol. Chem. 1998, 273 (14), 8308-8316, and Zhang, X.; Zhang, B.; Lin, J.; Wei, D. Oxidation of Ethylene Glycol to Glycolaldehyde Using a Highly Selective alcohol Dehydrogenase from Gluconobacter Oxydans. J. Mol. Catalysis B 2015, 112, 69-75.

Glycolaldehyde can also be derived from methanol via a metabolic pathway which successively uses the enzyme activities of the methanol dehydrogenase for the conversion of methanol into formaldehyde and the glycolaldehyde synthase for the conversion of formaldehyde into glycolaldehyde, as described in publication Lu, X.; Liu, Y.; Yang, Y.; Wang, S.; Wang, Q.; Wang, X.; Yan, Z.; Cheng, J.; Liu, C.; Yang, X.; et al. Constructing a Synthetic Pathway for Acetyl-Coenzyme A from One-Carbon through Enzyme Design. Nat Commun 2019, 10 (1), 1378.

Glycolaldehyde can also be derived from xylose via a multistage metabolic pathway, which successively uses the enzyme activities of xylose isomerase for the conversion of D-xylose into D-xylulose, of xylulose-1-kinase for the conversion of D-xylulose into D-xylulose-1P and of xylulose-1P-aldolase for the conversion of xylose-1P-aldolase into glycolaldehyde, known from Cam et al./2016/ACS Synth Biol/5/607-61.

One advantage of using methanol is that, just like ethylene glycol, it can easily be derived from synthesis gas. The biosynthesis of threonine or HMTB via DHB from ethylene glycol can therefore rightly be described as a particularly sustainable production method.

Further details, features and advantages of embodiments of the invention result from the figures and the following description of exemplary embodiments. Wherein

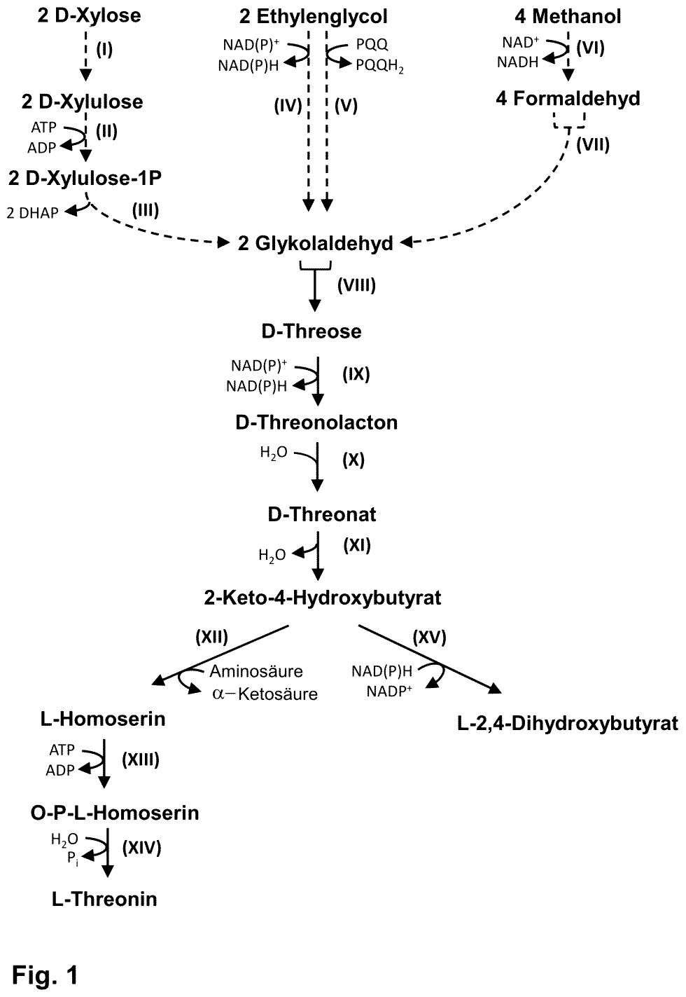

FIG. 1: shows a schematic representation of a draft of a five-stage metabolic pathway for the conversion of glycolaldehyde to DHB,

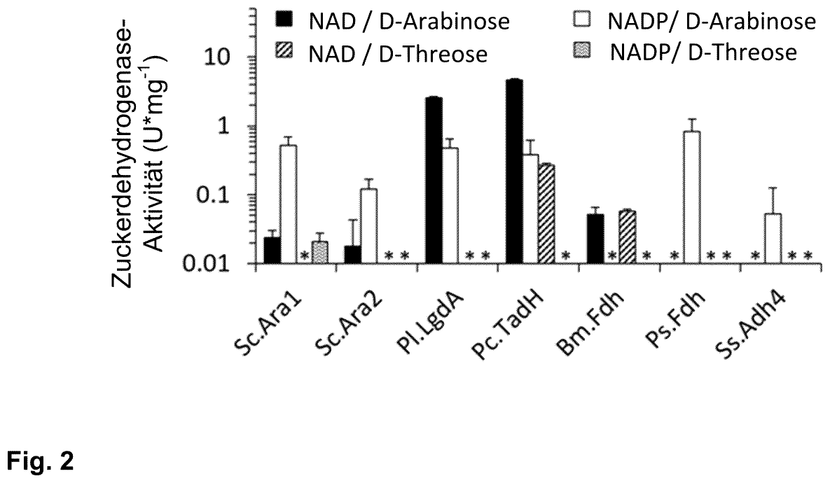

FIG. 2: shows a column diagram with the results of the screening of candidate enzymes for NAD(P)-dependent D-threose dehydrogenase activity,

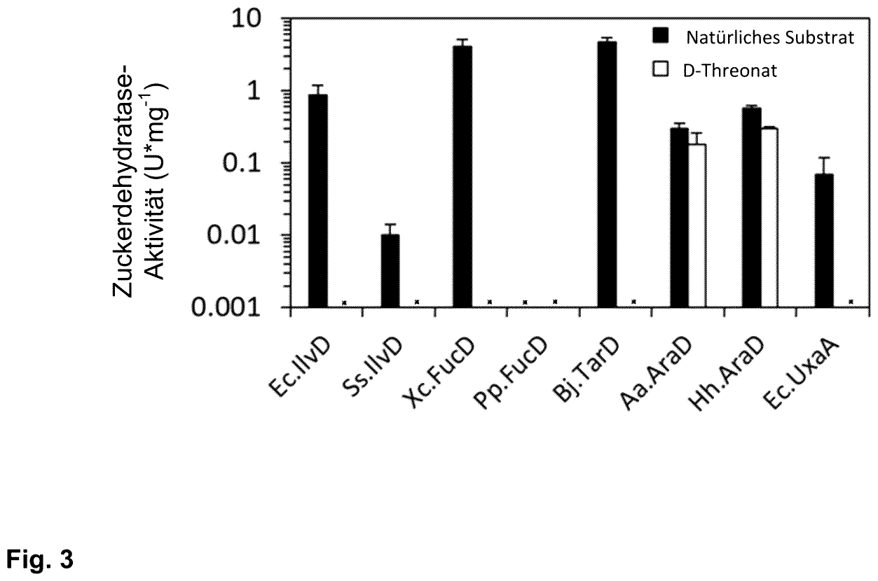

FIG. 3: shows a column diagram with the results of the screening of candidate enzymes for D-threonate dehydratase activity,

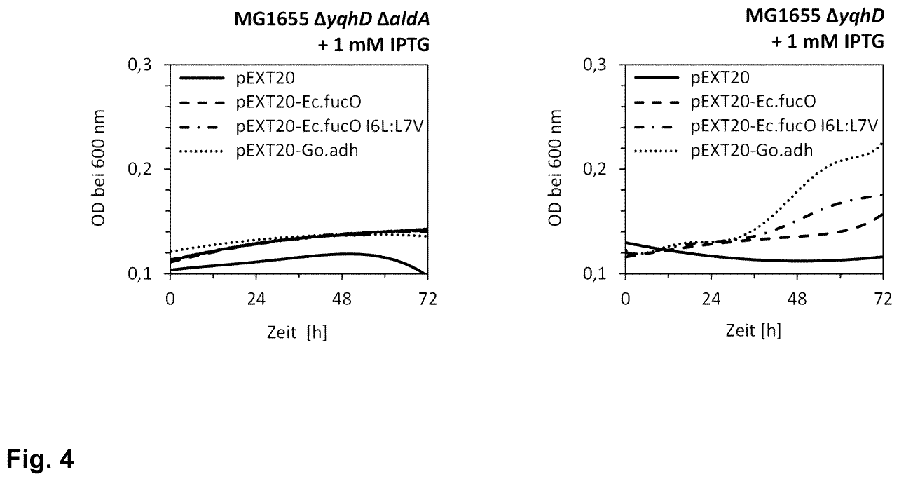

FIG. 4: shows a diagram for representing the growth pattern of E. coli strains expressing various ethylene glycol dehydrogenases, and

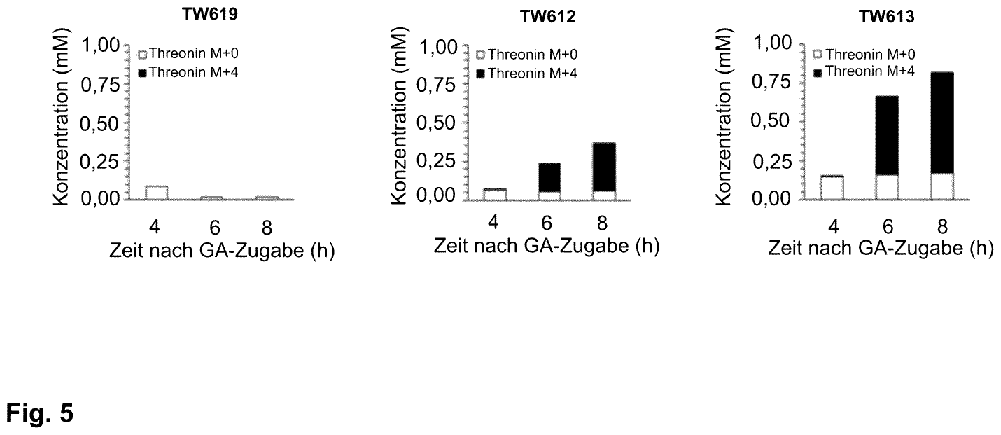

FIG. 5: shows column diagrams with the results of a ¹³C-based metabolic flow analysis showing the biosynthesis of L-threonine from glycolaldehyde (GA) via the synthetic metabolic pathway.

In FIG. 1, various methods for the preparation of 2,4-dihydroxybutyrate (DHB) or L-threonine from glycolaldehyde using microbial metabolic pathways are schematically represented, wherein all these microbial metabolic pathways have in common the four reaction stages which proceed in succession and are catalyzed by threose aldolase, threose dehydrogenase, threono-1,4-lactonase and threonate dehydratase.

The metabolic pathway is expressed in a microbial production strain, preferably of the type E. coli, which is modified beforehand with respect to its natural form (wild type) by introducing at least one of the genes necessary for the expression of said enzymes into the production strain.

In the case of the production of L-2,4-dihydroxybutyrate, two molecules of glycolaldehyde can be converted to 2-keto-4-hydroxybutyrate (OHB) by the above-mentioned four successive reaction stages and finally to L-2,4-dihydroxybutyrate (DHB) by a subsequent fifth reaction stage without loss of carbon.

In the case of the production of L-threonine, glycol aldehyde is first converted to 2-keto-4-hydroxybutyrate (OHB) by these four successive reaction stages, followed by a step of the enzymatic conversion of 2-keto-4-hydroxybutyrate to L-homoserine, by a step of the enzymatic conversion of L-homoserine to O-phospho-L-homoserine (O-P-L-homoserine) and by a step of the enzymatic conversion of O-phospho-L-homoserine to L-threonine.

Both metabolic pathways are compatible with the use of ethylene glycol, methanol or D-xylose as starting materials. Glycolaldehyde-producing reactions are shown as dashed arrows in FIG. 1. The different enzymes or enzyme activities of the metabolic pathways are indicated by Roman numerals in FIG. 1.

Glycolaldehyde can be derived from xylose via a multistage metabolic pathway, which uses the enzyme activities of xylose isomerase (I) in succession for the conversion of D-xylose to D-xylulose, xylulose-1-kinase (II) for the conversion of D-xylulose to D-xylulose-1P and xylulose-1P-aldolase (III) for the conversion of D-xylulose-1P to glycolaldehyde.

Glycolaldehyde can be derived from ethylene glycol via a metabolic pathway which uses either the enzyme activities of the PQQ-dependent ethylene glycol dehydrogenase (membrane-bound) (IV) or of the NAD(P)-dependent ethylene glycol dehydrogenase (cytosolic) (V) for the conversion of ethylene glycol.

Glycolaldehyde can be derived from methanol via a metabolic pathway which uses the enzyme activities of the methanol dehydrogenase (VI) for the conversion of methanol to formaldehyde and the glycol aldehyde synthase (VII) for the conversion of formaldehyde to glycol aldehyde in succession.

The production of the metabolic product DHB from glycolaldehyde in Escherichia coli was possible by designing a metabolic pathway with five successive reaction stages which are catalyzed by the enzyme activities of D-threose aldolase (VIII), D-threose dehydrogenase (IX), D-threono-1,4-lactonase (X), D-threonate dehydratase (XI) and OHB reductase (XV). In the first stage, two molecules of glycolaldehyde (GA) are bonded to form a molecule D-threose. The resulting four-carbon sugar is then oxidised by a D-threose dehydrogenase (IX) to D-threono-1,4-lactone, which is converted to the corresponding sugar acid or D-threonate in a reaction catalyzed by a D-threono-1,4-lactonase (X). In the last two enzymatic steps, D-threonate is dehydrated to OHB by a D-threonate dehydratase (XI), which is ultimately reduced to DHB in a reaction catalyzed by OHB reductase (XV).

In the production of L-threonine, glycol aldehyde is first converted to 2-keto-4-hydroxybutyrate (OHB) by the mentioned four reaction stages in succession, followed by a step of the enzymatic conversion of 2-keto-4-hydroxybutyrate to L-homoserine using an L-homoserine transaminase (XII), followed by a step of the enzymatic conversion of L-homoserine to O-phospho-L-homoserine with ATP consumption and using an L-homoserine kinase (XIII) and a step of the enzymatic conversion of O-phospho homoserine to L-threonine using an L-threonine synthase (XIV).

Most of the enzymatic activities mentioned were already known and the necessary genes, if not already contained in the production strain, could be introduced into the production strain in a suitable manner, but others had to be identified by screening.

Both D-threose aldolase and OHB reductase activities have already been described in literature. In particular, according to publication Szekrenyi, A.; Soler, A.; Garrabou, X.; Guérard-Hélaine, C.; Parella, T.; Joglar, J.; Lemaire, M.; Bujons, J.; Clapés, P. Engineering the Donor Selectivity of D-Fructose-6-Phosphate Aldolase for Biocatalytic Asymmetric Cross-Aldol Additions of Glycolaldehyde. Chemistry 2014, 20 (39), 12572-12583, in the case of D-fructose-6-phosphate aldolase from Escherichia coli (Ec. FsaA), the in vitro catalysis of the reversible enzymatic homo-aldol addition of glycolaldehyde to D-threose could already be shown. In addition, it was known that the mutated variant Ec. FsaA L107Y: A129G (Ec.FsaA^{TA}) has an activity which is increased by three orders of magnitude in comparison with the wild type for the production of D-threose. This mutated enzyme could therefore advantageously be used in the above-mentioned metabolic pathway. In addition, the mutated malate dehydrogenase Ec. Mdh^{5Q} obtained by the introduction of 5 point mutations in the L-malate dehydrogenase enzyme of E. coli (Ec. Mdh I12V:R81A:M85Q:D86S:G179D), was also described as highly active in publication Frazão, C. J. R.; Topham, C. M.; Malbert, Y.; François, J. M.; Walther, T. Rational Engineering of a Malate Dehydrogenase for Microbial Production of 2,4-Dihydroxybutyric Acid via Homoserine Pathway. Biochem. J. 2018, 475 (23), 3887-3901. This enzyme could therefore be selected as OHB reductase in order to catalyze the last conversion step of the DHB synthesis pathway.

By means of references from literature, it was also possible to determine an enzyme which, inter alia, also had D-threono-1,4-lactonase activity. Westlake, A. Thermostable Enzymes Important For Industrial Biotechnology. Date: Jun. 10, 2019, reported that the gluconolactonase from Thermogutta terrifontis, abbreviated here as Tt.Lac11, is active on a large plurality of lactones. The enzymes were also described as active on D-threono-1,4-lactone, although only a low reaction rate was reported. The kinetic properties were newly analyzed by the inventors and surprisingly comparable catalytic activities were determined for both the natural substrate L-Fucono-1,4-lactone and for D-Threono-1,4-lactone. Since the enzyme has a high affinity for D-threono-1,4-lactone (Km=2.92 mM), it is suitable for the metabolic pathway used according to the invention.

Several enzymes are known which catalyze a NAD-dependent oxidation of ethylene glycol to glycol aldehyde. These include the 1,2-propanediol dehydrogenase from E. coli (Ec.FucO), see Boronat, A.; Caballero, E.; Aguilar, J. Experimental Evolution of a Metabolic Pathway for Ethylene Glycol Utilization by Escherichia Coli. J. Bacteriol. 1983, 153 (1), 134-139, and the alcohol dehydrogenase GOX0313 from Gluconobacter oxidans (Go.Adh), see Zhang, X.; Zhang, B.; Lin, J.; Wei, D. Oxidation of Ethylene Glycol to Glycolaldehyde Using a Highly Selective alcohol Dehydrogenase from Gluconobacter Oxydans. J. Mol. Catalysis B 2015, 112, 69-75. Furthermore, it is known that through the mutation of the Ec. FucO in the positions Ile6Leu and Leu7Val, a higher oxygen resistance of the resulting enzyme (Ec.FucO 16L: L7V) can be achieved, see Lu, Z.; Cabiscol, E.; Obradors, N.; Tamarit, J.; Ros, J.; Aguilar, J.; Lin, E. C. Evolution of an Escherichia Coli Protein with Increased Resistance to Oxidative Stress. J. Biol. Chem. 1998, 273 (14), 8308-8316. The resulting enzyme is also known under the name Ec. FucO^{OR} wherein OR is the abbreviation for oxygen resistant.

As enzymes with L-homoserine transaminase activity for step (XII) of the enzymatic conversion of 2-keto-4-hydroxybutyrate to L-homoserine, aspartate aminotransferase from E. coli (Ec. AspC) and glutamate-pyruvate aminotransferase of the mutated variant Ec.AlaC A142P: Y275D are known, see Bouzon, M.; Perret, A.; Loreau, O.; Delmas, V.; Perchat, N.; Weissenbach, J.; Taran, F.; Marliére, P. A Synthetic Alternative to Canonical One-Carbon Metabolism. ACS Synth Biol 2017, 6 (8), 1520-1533. Enzymes with L-homoserine kinase activity for the step (XIII) of converting L-homoserine to O-phospho-L-homoserine are also known, in particular homoserine kinase from E. coli (EcThrB). Threonine synthase from E. coli (Ec. ThrC) has L-threonine synthase activity for step (XIV) of the enzymatic conversion of O-phospho-L-homoserine to L-threonine.

Of the enzyme activities of the schematically represented metabolic pathway mentioned above and represented in FIG. 1, those with D-threose dehydrogenase activity (IX) and D-threonate dehydratase activity (XI) had not yet been described. By screening a selection of candidate enzymes, however, such activities could be identified.

Concerning the materials and methods used, the following should be noted: All chemicals and solvents were purchased from Sigma-Aldrich, unless other companies are stated. The restriction endonucleases and the DNA-modifying enzymes were acquired from the company New England Biolabs (NEB) and employed in accordance with the manufacturer's instructions. The DNA plasmid isolation was carried out by means of a Monarch® plasmid miniprep kit from the company NEB. The DNA extraction from the agarose gel and the purification of the product of the polymerase chain reaction (PCR), a method for multiplying the genetic substance (DNA) in vitro, were carried out by means of the Monarch® DNA gel extraction kit from the company NEB. DNA sequencing was performed by the company Eurofins SAS (Ebersberg, Germany).

All plasmids and host strains constructed and employed for the studies are listed in Table 1. The primers are listed in Table 2 and in Table 12.

TABLE 1
Used strains and plasmids

| Name | Description | |
|---|---|---|
| of strain | Genotype | Origin |
| DH5α | E. coli fhuA2 Δ(argF-lacZ)U169 phoA glnV44 | NEB |
| | Φ80 Δ(lacZ)M15 gyrA96 recA1 relA1 endA1 | |
| | thi-1 hsdR17 | |
| BL21(DE3) | E. coli fhuA2 [Ion] ompT gal (λ DE3) [dcm] | NEB |
| | ΔhsdS λ DE3 = λ sBamHlo ΔEcoRI- | |
| | Bint::(lacl::PlacUV5::T7 gene1) i21 Δnin5) | |
| MG1655 | E. coli F- λ- ilvG- rfb-50 rph-1 | ATCC ® |
| | | 47076 ™ |
| TW63 | MG1655 ΔyqhD | JMF |
| TW64 | MG1655 ΔyqhD ΔaldA | JMF |
| TW145 | TW64/pEXT20-Ec.fsaA^{TA} | constructed |
| TW146 | TW64/pEXT20-Ec.fsaA^{TA}-Pc.tadH | constructed |
| TW253 | TW64/pACT3-Ec.fsaA^{TA}-Pc.tadH | constructed |
| TW334 | TW64/pEXT22/pEXT21 | constructed |
| TW336 | TW64/pEXT22/pEXT21-Re.kdgT | constructed |
| TW335 | TW64/pEXT22-Ec.mdh^{5Q}/pEXT21-Re.kdgT | constructed |
| TW338 | TW64/pEXT22-Ec.mdh^{5Q}-Aa.araD/pEXT21- | constructed |
| | Re.kdgT | |
| TW339 | TW64/pEXT22-Ec.mdh^{5Q}-Hh.araD/pEXT21- | constructed |
| | Re.kdgT | |
| TW337 | TW64/pEXT22-Ec.mdh^{5Q}-Hh.araD/pEXT21 | constructed |
| TW288 | TW253/pEXT22 | constructed |
| TW290 | TW253/pEXT22-Ec.mdh^{5Q}-Hh.araD | constructed |
| TW293 | TW64 pACT3-Ec.fsaA^{TA}-Pc.tadH- | constructed |
| | Tt.lac11 pEXT22-Ec.mdh^{5Q}-Hh.araD | |
| TW304 | TW64 pACT3-Ec.fsaA^{TA}-Pc.tadH- | constructed |
| | Tt.lac11 pEXT22-Ec.mdh^{5Q}-Hh.araD pEXT21- | |
| | Re.kdgT | |
| TW354 | TW64 pACT3-Ec.fsaA^{TA}-Pc.tadH- | constructed |
| | Tt.lac11^{v1} pEXT22-Ec.mdh^{5Q}- | |
| | Hh.araD pEXT21-Re.kdgT | |
| TW363 | TW64 pACT3-Go.adh-Ec.fsaA^{TA}-Pc.tadH- | constructed |
| | Tt.lac11^{v1}pEXT22-Ec.mdh^{5Q}-Hh.araD | |
| | pEXT21-Re.kdgT | |
| TW444 | TW64 + pACT3-Ec.fsaA^{TA}-Aa.tadH- | constructed |
| | Tt.lac11^{v1} pEXT22-Ec.mdh^{5Q}- | |
| | Hh.araD pEXT21-Re.kdgT | |
| TW445 | TW64 + pACT3-Ec.fsaA^{TA}-Xc.Fdh- | constructed |
| | Tt.lac11v1 pEXT22-Ec.mdh^{5Q}- | |
| | Hh.araD pEXT21-Re.kdgT | |
| TW446 | TW64 + pACT3-Ec.fsaA^{TA}-Ppi.TadH- | constructed |
| | Tt.lac11v1 pEXT22-Ec.mdh^{5Q}- | |
| | Hh.araD pEXT21-Re.kdgT | |
| TW452 | TW64 + pACT3-Ec.fsaA^{TA}-Ppi.TadH- | constructed |
| | Tt.lac11v1 pEXT22-Ec.mdh5Q- Hh.araDC434S | |
| TW453 | TW64 + pACT3-Ec.fsaA^{TA}-Ppi.TadH- | constructed |
| | Tt.lac11v1 pEXT22-Ec.mdh^{5Q}- | |
| | Ca.araD pEXT21-Re.kdgT | |
| TW454 | TW64 + pACT3-Ec.fsaA^{TA}-Ppi.TadH- | constructed |
| | Tt.lac11v1 pEXT22-Ec.mdh5Q- | |
| | Pm.araD pEXT21-Re.kdgT | |
| TW559 | TW64 + pACT3-Ec.fsaA^{TA}- | constructed |
| | Tt.lac11v1 pEXT22- Ec.mdh5Q-Pm.araD pEXT21- | |
| | Re.kdgT | |
| TW462 | MG1655 ΔyqhD ΔaldA thrBC^{proD} rhtB^{proD} | constructed |
| TW469 | TW64 pACT3-Ec.fsaA^{TA}-Pc.tadH- | constructed |
| | Tt.lac11^{v1} pEXT22-Ec.mdh^{7Q}- | |
| | Hh.araD pEXT21-Re.kdgT | |
| TW612 | TW64 pEXT22-Ec.aspC-Hh.araD | constructed |
| | pEXT21-Re.kdgT | |
| | pACT3-Ec.fsaA^{TA}-Pc.tadH-Tt.lac11^{v1} | |
| TW613 | TW462 + pEXT22-Ec.aspC-Hh.araD + | constructed |
| | pEXT21-Re.kdgT + | |
| | pACT3-Ec.fsaA^{TA}-Pc.tadH-Tt.lac11^{v1} | |
| TW619 | TW64 + pEXT22 leer + | constructed |
| | pEXT21-Re.kdgT + | |
| | pACT3-Ec.fsaA^{TA}-Pc.tadH-Tt.lac11^{v1} | |

| Name | Description | |
|---|---|---|
| of plasmid | relevant characteristic | |
| pET28a(+) | Kan^{R}, f1 ori; IPTG-inducible promoter T7 | Novagen ™ |
| pEXT22 | Kan^{R}, R100 ori; IPTG-inducible tac promoter | (Dykxhoorn et |
| | | al, 1996) |
| pACT3 | ChmR; p15A ori; IPTG-inducible tac promoter | (Dykxhoorn et |
| | | al, 1996) |
| pEXT20 | AmpR; colE1 ori; IPTG-inducible tac promoter | (Dykxhoorn et |
| | | al, 1996) |
| pEXT20-Ec.fsaA | pEXT20 derivative which carries Ec.fsaA | constructed |
| pEXT20- | pEXT20 derivative which carries | constructed |
| Ec.fsaA^{TA} | Ec.fsaAL107Y:A129G | |
| pEXT20- | pEXT20 derivative which carries | constructed |
| Ec.fsaA^{TA}- | Ec.fsaAL107Y:A129G, Pc.tadH (codon- | |
| Pc.tadH | optimized) | |
| pACT3- | pACT3 derivative which carries | constructed |
| Ec.fsaA^{TA}- | Ec.fsaAL107Y:A129G, Pc.tadH (codon- | |
| Pc.tadH | optimized) | |
| pACT3- | pACT3 derivative which carries | constructed |
| Ec.fsaA^{TA}- | Ec.fsaAL107Y:A129G, Pc.tadH (codon- | |
| Pc.tadH-Tt.lac11 | optimized); Tt-lac11 (codon-optimized) | |
| pACT3- | pACT3 derivative which carries | constructed |
| Ec.fsaA^{TA}- | Ec.fsaAL107Y:A129G, Pc.tadH (codon- | |
| Pc.tadH- | optimized); Tt-lac11 (codon-optimized; Δ1-38 | |
| Tt.lac11^{v1} | aa) | |
| pACT3-Go.adh- | pACT3 derivative which carries | constructed |
| Ec.fsaA^{TA}- | Ec.fsaAL107Y:A129G, Pc.tadH (codon- | |
| Pc.tadH- | optimized); Tt-lac11 (codon-optimized; Δ1-38 | |
| Tt.lac11^{v1} | aa) | |
| pEXT22- | pEXT22 derivative which carries Ec.mdh^{5Q} | constructed |
| Ec.mdh^{5Q} | (=Ec.mdhl12V:R81A:M85Q:D86S:G179D) | |
| pEXT22- | pEXT22 derivative which carries | constructed |
| Ec.mdh^{5Q}- | Ec.mdh^{5Q}, Hh.araD (=Hh.e2k99_19880) | |
| Hh.araD | | |
| pEXT22- | pEXT22 derivative which carries Ec.mdh^{5Q} | constructed |
| Ec.mdh^{5Q}- | and the Cys434Ser mutant of Hh.araD | |
| Hh.araDC434S | (=Hh.e2k99_19880) | |
| pEXT22- | pEXT22 derivative which carries | constructed |
| Ec.mdh^{5Q}- | Ec.mdh^{5Q}, Ca.araD | |
| Ca.araD | | |
| pEXT22- | pEXT22 derivative which carries | constructed |
| Ec.mdh^{5Q}- | Ec.mdh^{5Q}, Pm.araD | |
| Pm.araD | | |
| pEXT22- | pEXT22 derivative which carries Ec.mdh^{5Q}, | constructed |
| Ec.mdh^{5Q}- | Aa.araD | |
| Aa.araD | (=Aa.acav1654) | |
| pEXT22- | pEXT22 derivative which carries Ec.mdh^{7Q}, | constructed |
| Ec.mdh^{7Q}- | Hh.araD | |
| Hh.araD | (=Hh.e2k99_19880) | |
| pEXT22- | pEXT22 derivative which carries Ec.aspC, | constructed |
| Ec.aspC- | Hh.araD | |
| Hh.araD | (=Hh.e2k99_19880) | |

TABLE 2
Sequences of used primers

| Primer | Sequence (5′-3′) |
|---|---|
| 222 (fw_xhoi_Ecfuco*) | taagcactcgaggtttaactttaagaaggagatataccATGGCT |
| | AACAGAATGCTGGTGA |
| | (SEQ ID No. 1) |
| 223 (rv_Ecfuco_xbai) | tgcttaTCTAGATTACCAGGCGGTATGGTAAAGC |
| | (SEQ ID No. 2) |
| 224 (fw_xhoi_Ecfuco) | taagcactcgaggtttaactttaagaaggagatataccatgGCTA |
| | ACAGAATGATTCTGA |
| | (SEQ ID No. 3) |
| 226 (rv_gox0313_xbai) | tgcttaTCTAGATTAGGACCGGAAGTCGA |
| | (SEQ ID No. 4) |
| 315 | taagcactcgaggtttaactttaagaaggagatataccATGGCT |
| (fw_xhoi_rbs+gox0313) | GATACAATGCTC |
| | (SEQ ID No. 5) |
| 209 (fw_xbai_pzs13) | taagcactcgagtgtgtgaaattgttatccg |
| | (SEQ ID No. 6) |
| 284 | TTCGAGCTCGGTACCC |
| (pext20_MCSpos1_fw) | (SEQ ID No. 7) |
| 326 (bamhi_fsaA^{TA}_fw) | taagcaGGATCCGtttaactttaagaaggagatataccATGG |
| | AACTGTATCTGGATACTTCAG |
| | (SEQ ID No. 8) |
| 327 (Rv_Ecfsa_xbai) | tgcttaTCTAGATTAAATCGACGTTCTGCCAAAC |
| | (SEQ ID No. 9) |
| 328 (fsaA^{TA}_swai_rv) | tgcttaATTTAAATTTAAATCGACGTTCTGCCAAAC |
| | (SEQ ID No. 10) |
| 303 (Swai_tadH_fw) | taagcaATTTAAATGtttaactttaagaaggagatataccATG |
| | TCTACCGATAGTTTACAACAG |
| | (SEQ ID No. 11) |
| 304 (Tadh_xbaI_rv) | tgcttaTCTAGATTATGCCGGAACCGGTG |
| | (SEQ-ID-No. 12) |
| 313 (Kpnl_gox0313_f) | taagcaGGTACCgtttaactttaagaaggagatataccATGG |
| | CTGATACAATGCTC |
| | (SEQ ID No. 13) |
| 314 (BamHl_gox0313_r) | tgcttaGGATCCTTAGGACCGGAAGTCGAG (SEQ |
| | ID No. 14) |
| 438(xbai_Tt- | taagcaGGTACCgtttaactttaagaaggagatataccATGC |
| thte1497opt_fw) | GCAAACTGCTTGGCAG |
| | (SEQ ID No. 15) |
| 439 (Tt- | tgcttagtcgaCTTAGAACCCCAGTCCTTTGGTTTTC |
| thte1497opt_sali_rv) | G |
| | (SEQ ID No. 16) |
| 305 (Sacl_ecmdh5q_fw) | taagcaGAGCTCGtttaactttaagaaggagatataccATGA |
| | AAGTCGCAGTCCTC |
| | (SEQ ID No. 17) |
| 258 (Rv_ecmdh5q_bamhi) | tgcttaGGATCCTTACTTATTAACGAACTCTTCGC |
| | (SEQ ID No. 18) |
| 551 (bamhi_acav1654_fw) | taagcaGGATCCGtttaactttaagaaggagatataccATGT |
| | CGACTGATGCACTGGC |
| | (SEQ ID No. 19) |
| 552 (Rv_acav1654_xbai) | tgcttaTctagaTCACATCACCGCGCCGAG |
| | (SEQ ID No. 20) |
| 553 (bamhi_hh-e2k99_fw) | taagcaGGATCCGtttaactttaagaaggagatatacc |
| | ATGAAAGCCAACTCTCCCG (SEQ |
| | ID No. 21) |
| 554 (Rv_hh-e2k99_xbai) | tgctta TctagaTCAGGTGTAGACGCCGATG |
| | (SEQ ID No. 22) |
| 454 (bamhi_kdgt_fw) | taagcaggatccgtttaactttaagaaggagatataccATGCAG |
| | ATTTCTATCAAACGCGCCA |
| | (SEQ ID No. 23) |
| 455 (hindiii_kdgt_rv) | tgcttaAagcttTCATGCGGCGGTCCTCA |
| | (SEQ ID No. 24) |
| 667 (Swai_aa-tadH_fw) | taagcaATTTAAATGtttaactttaagaaggagatataccATG |
| | AAGGTCACAGAAACACGCC |
| | (SEQ ID No. 25) |
| 668 (Aa-Tadh_xbaI_rv) | tgcttaTCTAGATCATGGCGCGGCTCCG |
| | (SEQ ID No. 26) |
| 671 (Swai_Xc-fdH_fw) | taagcaATTTAAATGtttaactttaagaaggagatataccATG |
| | AATACACGTCGCCAATTCCTGTCTG |
| | (SEQ ID No. 27) |
| 672 (Xc-fdh_xbaI_rv) | tgcttaTCTAGATCATCCAGCCGCCGGCAC |
| | (SEQ ID No. 28) |
| 716 (Swai_ppi.tadh_fw) | taagcaATTTAAATGtttaactttaagaaggagatataccATG |
| | AATCGGCGCACAGG |
| | (SEQ ID No. 29) |
| 717 (Ppi.tadh_xbaI_rv) | tgcttaTCTAGACTAAACGGGCTCGTGTGT |
| | (SEQ ID No. 30) |
| 718 (Sdm-hh.e2k99- | GGTTCTGCCTATGGCAGCGCACCGGCACCGTC |
| C434S_fw) | G (SEQ ID No. 31) |
| 719 (Sdm-hh.e2k99- | CGACGGTGCCGGTGCGCTGCCATAGGCAGAA |
| C434S_rv) | CC (SEQ ID No. 32) |
| 724 (bamhi_Ca.araD_fw) | taagcaGGATCCGtttaactttaagaaggagatataccATGA |
| | AAAATGTTATAAAGATAAATGAAAAAGATAATG |
| | (SEQ ID No. 33) |
| 725 (Rv_Ca.araD_xbai) | tgcttaTCTAGATTATAGTGTTACACCGTTTTTAAATA |
| | TAGATATTTC (SEQ-ID-No. 34) |
| 732 (bamhi_pm.araD_fw) | taagcaGGATCCGtttaactttaagaaggagatataccATGA |
| | AGACCTCAACAGCAGAC |
| | (SEQ ID No. 35) |
| 733 (Rv_pm.araD_xbai) | tgcttaTctagaTCAGGTGATCGCGCCGATC |
| | (SEQ ID No. 36) |
| 622 (thrB-KAN-fw) | TGTCTTTGCTGATCTGCTACGTACCCTCTCATG |
| | GAAGTTAGGAGTCTGACGTGTAGGCTGGAGCT |
| | GCTTC |
| | (SEQ ID No. 37) |
| 623 (thrB-KAN-rev) | GATAGGGACGACGTGGTGTTAGCTGTGCATAT |
| | GAATATCCTCCTTAG |
| | (SEQ ID No. 38) |
| 624 (kan-proD-fw) | CACAGCTAACACCACGTCGT |
| | (SEQ ID No. 39) |
| 625 (thrB-proD-rev) | AACCCGACGCTCATATTGGCACTGGAAGCCGG |
| | GGCATAAACTTTAACcatATAATACCTCCTAAAGT |
| | TAAACAAAATTATTTGTAG |
| | (SEQ ID No. 40) |
| 626 (ver_proD-thrB_fw) | ATTGCCGAAGTGGATGGTAA |
| | (SEQ ID No. 41) |
| 627 (ver_proD-thrB_rev) | GTGACTACATCTCCGAGCAA |
| | (SEQ ID No. 42) |
| 752 (rhtB-KAN-fw) | TGCGACAGTAGCGTATTGTGGCACAAAAATAGA |
| | CACACCGGGAGTTCATCGTGTAGGCTGGAGCT |
| | GCTTC |
| | (SEQ ID No. 43) |
| 753 (rhtB-proD-rev) | CTTAAAATGATCGATGTCAGCAGGTAGGCAAAC |
| | CACCATTCTAAGGTcatATAATACCTCCTAAAGTT |
| | AAACAAAATTATTTGTAG |
| | (SEQ ID No. 44) |
| 754 (ver_proD-rhtB_fw) | CATGGTAAAAGCAGCAAACGCGT |
| | (SEQ ID No. 45) |
| 755 (ver_proD-rhtB_rev) | TATGAATCGCCAGTCCGGTCTGA |
| | (SEQ ID No. 46) |
| 805 (Sacl_ECaspC_fw) | TAAGCAGAGCTCGTTTAACTTTAAGAAGGAGAT |
| | ATACCATGTTTGAGAACATTACCGC |
| | (SEQ ID No. 47) |
| 806 (Rv_EcaspC_bamhi) | TGCTTAGGATCCTTACAGCACTGCCACAATC |
| | (SEQ ID No. 48) |

In Table 2, the restriction sites are underlined in the primer sequences, the coding start/stop sequences are bold and the RBS sequences are marked in italics. The plasmids pEXT20-Ec.fucO, pEXT20-Ec.fucOl6L: L7V and pEXT20-Go.adh were constructed by PCR amplification of the Ec.fucO wild type, Ec.fucO16L: L7V and of the codon-optimized Go.adh gene using the primer pairs 224/223, 222/223 and 315/226, respectively. Genomic DNA from Escherichia coli MG1655 and synthetic genes served as template DNA for genes derived from Ec.fucO and Go.gox0313. All primers introduced certain restriction sites which flanked the respective genes. In addition, the primers introduced a ribosome binding sequence (RBS) immediately before the coding sequence.

Escherichia coli K-12 substr. MG1655 ΔyqhD ΔaldA was used as the starting strain for the construction of threonine-producing strains. The expression of the endogenous thrBC and rhtB genes was made constitutive by replacing the native chromosomal 5′-UTR of each operon or gene by the synthetic constitutive and isolated promoter proD Davis, J. H.; Rubin, A. J.; Sauer, R. T.: Design, construction and characterization of a set of insulated bacterial promoters. In: Nucleic Acid Res., 2011, 3, pp. 1131-1141. The proD sequence was preceded by a chloramphenicol resistance cassette (FRT-cat-FRT-PproD), the elements of which were first amplified from the plasmids pTOPO-proD and pKD with the primers listed in Table 2. The PCR products were digested with Dpnl, purified and assembled by fusion PCR using primers which had a homology of about 50 bp to the flanking region of the genomic target locus. The resulting DNA fragment was transformed into the respective target strains which expressed the A-red recombinase from the pKD46 plasmid in order to replace the natural gene promoter in these strains. Chloramphenicol-resistant clones were selected on LBagar plates which were enriched with the antibiotic and it was confirmed by PCR analysis (primers see Table 2) that they contained the corresponding insert size. The integrated promoter sequences were checked for correct sequencing by DNA sequencing. The cat cassette was removed from the genome by expressing FLP recombinase from the pCP20 plasmid Cherepanov P. P.; Wackernagel, W.: Gene disruption in Escherichia coli: TcR and KmR cassettes with the option of Flp-catalyzed excision of the antibiotic-resistance determinant. In: Gene, 1995, 158 (1), pp. 9-14, and the correct excision of the cassette was checked by PCR using locus-specific primers (Table 2). The plasmids were transformed into the target E. coli strains with the help of standard protocols.

The high-copy plasmid pEXT20 was amplified using the primer pair 209/284. The PCR products were digested with Xhol/Xbal restriction enzymes and ligated into the vector backbone using T4 DNA ligase (company NEB).

The plasmids pEXT20-Ec.fsaA and pEXT20-Ec.fsaA^{TA} were constructed by amplification of the Ec.fsaA wild type and Ec.fsaAL107Y: A129G genes using the primer pair 326/327. The genomic DNA of Escherichia coli MG1655 and synthetic genes served as template DNA. The resulting PCR products and the pEXT20 expression vector were digested with BamHI/Xbal and ligated. The plasmid Ec.fsaA^{TA}-Pc.tadH was constructed by PCR amplification of Ec-fsaAL107Y: A129G and of the codon-optimized synthetic Pc.tadH gene using the primer pairs 326/328 and 303/304, respectively. The resulting PCR products were each digested with BamHI/Swal and Swal/Xbai restriction enzymes and ligated into the pEXT20 vector digested with BamHI/Xbal.

A similar procedure was used for the construction of pACT3-Ec.fsaA^{TA}-Pc.tadH, but in which the medium-copy plasmid pACT3 served as the backbone. The plasmid pACT3-Ec.fsaA^{TA}-Pc.tadH-Tt.lac11 was constructed by amplification of the codon-optimized synthetic Tt.lac11 gene using the primer pair 438/439. The PCR product and the pACT3-Ec.fsaA^{TA}-Pc.tadH vector were then digested with Xbal/Sall restriction enzymes and ligated. Shorter versions of Tt.thte1497op genes where the signal sequence for the periplasmatic export at the N-terminus (1:115-1,068 nt; 2:154-1.068 nt) is missing, were also amplified with PCR.

For the construction of the plasmids pACT3-Ec.fsaA^{TA}-Aa.tadH-Tt.lac11^{V1}, pACT3-Ec.fsaA^{TA}-Xc.fdh-Tt.lac11^{V1} and pACT3-Ec.fsaA^{TA}-Ppi.tadH-Tt.lac11^{V1}, the genes Aa.tadH, Xc.fdh and Ppi.tadH were PCR-amplified with the help of the primer pairs 667/668, 671/672 or 716/717. Genomic DNAs of the strains A. avenae DSM7227, X. campestris DSM3586 and a synthetic gene for Ppi.tadH served as template. The resulting PCR products and the vector pACT3-Ec.fsaA^{TA}-Pc.tadH-Tt.lac11^{V1} were digested with Swal/Xbal and then ligated.

For the construction of the plasmid pACT3-Go.adh-Ec.fsaA^{TA}-Pc.tadH-Tt.lac11^{V1}, the genes Go.adh, Ec.fsaA^{TA}, Pc.tadH and Tt.lac11^{V1} were amplified with the help of the primer pairs 313/314, 326/328, 303/304 and 600/439. The resulting PCR products were digested with Kpnl/BamHI, Bamhl/Swal, Swal/Xbal or Xbal/Sall and ligated into the pACT3 vector digested in Kpnl/Sall.

The plasmid pEXT22-Ec.mdh^{5Q} was constructed by PCR amplification of the OBH reductase coding gene Ec.mdh^{5Q} (synthetic gene) using the primer pair 305/258. The resulting PCR product and the low-copy vector pEXT22 were then digested with Sacl/Bamhl and litigated. The plasmid pEXT22-Ec.mdh^{7Q} was constructed by PCR amplification of the gene Ec.mdh^{7Q} encoding OHB reductase, produced by mutation of Ec.mdh^{5Q} as described below, using the primer pair 305/258. The plasmid pET28-Ec.mdh^{7Q} served as template DNA. The resulting PCR product and the low-copy vector pEXT22 were then digested with Sacl/Bamhl and ligated.

The plasmids pEXT22-Ec.mdh^{5Q}-Aa.araD, pEXT22-Ec.mdh^{5Q}. Hh.araD and pEXT22-Ec.mdh^{7Q}-Hh.araD were constructed by amplification of Aa.araD and Hh.araD genes using the primer pairs 551/552 and 553/554, respectively. Genomic DNA of Acidovorax avenae DSM7227 and Herbaspirillum huttiense DSM10281 were used as the respective template DNAs. The resulting PCR products were digested with BamHI/Xbal and ligated individually into the corresponding sites in the vectors pEXT22-Ec.mdh^{5Q} or pEXT22-Ec.mdh^{7Q} digested with BamHI/Xbal. The plasmid pEXT21-Re.kdgT was constructed by amplification of the Re.kdgT gene from the genomic DNA of Cupriavidus necator H16 DSM428 using the primer pair 454/455. The PCR product and the pEXT21 vector backbone were digested with BamHI/Hindlll restriction enzymes and ligated.

For the construction of the plasmid pEXT22-Ec.aspC-Hh.araD, the genes Ec.aspC and Hh.araD were first PCR-amplified with the primer pairs 805/806 or 553/554. The used primers are listed in Table 1. Genomic DNA of E. coli MG1655 and Herbaspirillum huttiense DSM10281 were used as corresponding templates. The resulting PCR products were digested with Sacl/BamHI or BamHI/Xbal and ligated individually into the corresponding sites in the vector pEXT22 digested with Sacl/Xbal.

The plasmid pEXT22-Ec.mdh^{5Q}-Hh.araDC434S was constructed by inverse PCR on the template pEXT22-Ec.mdh^{5Q}-Hh.araD, which generated a mutation from cysteine to serine in position 434 using the primer pair 718/719. For the construction of the plasmids pEXT22-Ec.mdh^{5Q}-Ca.araD and pEXT22-Ec.mdh^{5Q}-Pm.araD, the genes Ca.araD and Pm.araD were PCR-amplified using the primer pairs 724/725 and 732/733, respectively. The genomic DNAs of Clostridium acetobutylicum DSM1731 Paraburkholderia mimosarum DSM21841 served as templates. The resulting PCR products and the plasmid pEXT22-Ec.mdh^{5Q}-Hh.araD were digested with BamHI/Xbal, purified and ligated.

All resulting constructions were transferred into chemically competent E. coli cells (DH5α, NEB) and verified by DNA sequencing with respect to the correct incorporation of the target genes. The plasmids were then transformed into the respective E. coli strain selected as production strain using standard methods, as known from Sambrook, J.; Fritsch, E. F.; Maniatis, T. Molecular Cloning: A Laboratory Manual. Mol. cloning a Lab. manual. 1989, No. Ed. 2.

In the following, the enzymatic tests are described: The protein concentrations were determined by the method of Bradfort (Roti®-Quant, Roth) before the enzymatic tests (assays). Unless otherwise described, all enzymatic tests were carried out for 20 minutes at 37° C. in 96-well microtiter plates in a total volume of 250 μL. The maximum reaction rate (vmax) and the values for the Michaelis constant (Km) were determined by adapting the kinetic data for at least five different substrate concentrations to the Michaelis-Menten equation. Adaptation was by non-linear regression in Matlab® R2015a.

### Determination of the Activity of the Ethylene Glycol Dehydrogenase:

The enzyme activity was determined in the oxidative direction by measuring the reduction of NAD+ at 340 nm (ε=6.22 mM-1 cm-1) during the oxidation of ethylene glycol. The reaction mixture for the activity determination contained 100 mM sodium glycine (pH 9.5), 0.5 mM NAD+ and corresponding amounts of purified enzyme or of the crude protein extract. The reactions were started by the addition of corresponding concentrations of substrate. A unit U of the ethylene glycol dehydrogenase activity was defined as the amount of enzyme which catalyzes the conversion of 1.0 μmol of NAD+ per minute.

### Determination of the Activity of D-Threose Aldolase:

The enzyme activity was determined by coupling the homo-aldol addition of glycolaldehyde with the NAD-dependent oxidation of D-threose, catalyzed by purified D-threo-aldose-1-dehydrogenase from Paraburkholderia caryophylli (Pc.TadH). The reaction mixture for the activity determination contained 60 mM 2-(4-(2-hydroxyethyl)-1-piperazinyl)-ethanesulfonic acid (HEPES) with pH 8, 10 mM NAD+, 100 μg mL-1 auxiliary enzyme and suitable amounts of purified enzyme or of the crude protein extract. The reactions were started by the addition of corresponding concentrations of substrate. A unit U of D-threose aldolase activity was defined as the amount of enzyme which catalyzes the conversion of 1.0 μmol of D-threose per minute.

### Determination of the Activity of the Sugar Dehydrogenase:

The enzyme activity was determined in the oxidative direction by measuring the reduction of NAD(P)+ at 340 nm during the oxidation of candidate sugars. The reaction mixture for the activity determination contained 50 mM HEPES (pH 8), 10 mM NAD(P)+ and suitable amounts of purified enzyme or of the crude protein extract. The reactions were started by the addition of various concentrations of (D)-arabinose or (D)-threose (Carbosynth, UK). A unit U of the sugar dehydrogenase activity was defined as the amount of enzyme which catalyzes the conversion of 1.0 μmol of sugar per minute.

### Determination of the Activity of the Lactonase:

The enzyme activity was carried out by measuring the concentration of protons which are released from the carboxylate product during the hydrolysis of lactones, using the colorimetric pH indicator bromothymol blue (ε=1.14 mM-1 cm-1) at 616 nm. The reaction mixture for the activity determination contained 2.5 mM HEPES (pH 7.1), 200 mM NaCl, 1% (v/v) DMSO, 0.1 mM bromothymol blue and suitable amounts of purified enzyme. The reaction was started by adding various amounts of (L)-fucono-1,4-lactone or (D)-threono-1,4-lactone. A unit U of the lactonase activity was defined as the amount of enzyme which catalyzes the hydrolysis of 1.0 μmol of lactone per minute.

### Determination of the Activity of the Sugar Acid Dehydratase:

The enzyme activity was determined by converting the 2-ketoacid reaction product to a semicarbazone whose concentration was measured at 250 nm. A calibration curve was obtained using pyruvate as 2-ketoacid (ε=2.24 mM-1 cm-1). The reaction mixture for the activity determination contained 60 mM HEPES (pH 7.3), 50 mM KCl, 10 mM MgCl2 and suitable amounts of purified enzyme or of the crude protein extract. The 1 mL reaction was started by addition of various sugar acids (L-fuconate, 2R-dihydroxyvalerate, D-altronate, D-tartrate, D-arabinonate or D-threonate) and incubated at 37° C. Aliquots of 200 μl were removed during the reaction after 0, 10, 20 and 40 minutes and admixed with 100 μl of 2 M HCl. The samples were then supplemented with 300 μl of semicarbazide solution (10 g*L⁻¹ semicarbazide hydrochloride and 15 g*L⁻¹ sodium acetate) and incubated at 30° C. for 10 minutes. Finally, 500 μL of distilled water were added to the derivatised product, wherein the absorption was measured immediately using a quartz cuvette. A unit U of the sugar acid dehydratase activity (U) was defined as the amount of enzyme which catalyzes the formation of 1.0 μmol of 2-ketoacid per minute.

### Determination of the Activity of the D-Threonate Dehydratase:

The enzyme activity was determined by coupling the dehydration of D-threonate with the NADH-dependent reduction of 2-keto-4-hydroxybutyrate (OHB) by purified OHB reductase Ec. Mdh^{5Q}, which is known from publication Frazão, C. J. R.; Topham, C. M.; Malbert, Y.; François, J. M.; Walther, T. Rational Engineering of a Malate Dehydrogenase for Microbial Production of 2,4-Dihydroxybutyric Acid via Homoserine Pathway. Biochem. J. 2018, 475 (23), 3887-3901. The reaction mixture contained 60 mM HEPES (pH 7.3), 50 mM KCl, 10 mM MgCl2, 0.25 mM NADH, 100 μg mL-1 auxiliary enzyme and suitable amounts of purified enzyme. The reaction was started by addition of different amounts of substrate. A unit U of the D-threonate dehydratase activity was defined as the amount of enzyme which catalyzes the formation of 1.0 μmol OHB per minute Candidate enzymes which were tested for D-threose dehydrogenase or D-threonate dehydratase activity are listed in Table 3.

### Determination of the Activity of the OHB Reductase:

The enzyme activity was determined in a reductive direction by measuring the oxidation of NAD(P)H at 340 nm during the reduction of OHB to DHB. The reaction mixture for the activity determination contained 60 mM HEPES (pH 7), 5 mM MgCl2, 50 mM KCl, 0.25 mM NADH or NADPH, 2 mM OHB and suitable amounts of purified enzyme. The reactions were started by adding OHB. A unit U of the OHB reductase activity was defined as the amount of enzyme which catalyzes the conversion of 1.0 μmol of NAD(P)H per minute. In order to determine the Km values for the co-factors and for OHB, the initial concentration of the one substrate was suitably varied, while the initial concentration of the other substrate was kept constant.

TABLE 3
Candidate enzymes tested for D-threose dehydrogenase and D-threonate dehydratase activity

| | | | | |
|---|---|---|---|---|
| | | Functional | UniProt | Codon |
| Enzymes | Origin | description | reference | optimiztion |

Candidate enzymes for D-threose dehydrogenases

| | | | | |
|---|---|---|---|---|
| Sc.Ara1 | Saccharomyces | NADP-dependent | D-P38115 | No |
| | cerevisiae | arabinose dehydrogenase | | |
| Sc.Ara2 | Saccharomyces | NAD-dependent D- | Q04212 | No |
| | cerevisiae | arabinose-1- dehydrogenase | | |
| Pc.TadH | Paraburkholderia | D-threo-aldose-1- | A0A1X7DDC2 | Yes |
| | caryophylli | dehydrogenase | | |
| Pl.LgdA | Paracoccus | Scyllo-inositol | K7ZP76 | Yes |
| | laeviglucosivorans | dehydrogenase | | |
| Ps.Fdh | Pseudomonas | D-threo-aldose-1- | Q52472 | Yes |
| | sp. 1143 | dehydrogenase | | |
| Xc.Fdh | Xanthomonas | L-fucose | A0A3E1KMH9 | No |
| | campestris | dehydrogenase | | |
| Aa.TadH | Acidovorax | D-threo-aldose-1- | F0Q4S3 | No |
| | avenae | dehydrogenase | | |
| Ppi.TadH | Paraburkholderia | D-threo-aldose-1- | A0A1N7S9V4 | Yes |
| | piptadeniae | dehydrogenase | | |
| Ss.Adh4 | Sulfolobus | D-arabinose | Q97YM2 | Yes |
| | solfataricus | dehydrogenase | | |
| Bm.Fdh | Burkholderia | L-fucose | A0A0H3KNE7 | No |
| | multivorans | dehydrogenase | | |

Candidate enzymes for D-threonate dehydratases

| | | | | |
|---|---|---|---|---|
| Ec.IlvD | Escherichia coli | Dihydroxy acid | P05791 | No |
| | | dehydratase | | |
| Ss.IlvD | Sulfolobus | Dihydroxy acid | Q97UB2 | Yes |
| | solfataricus | dehydratase | | |
| Xc.FucD | Xanthomonas | L-fuconate | Q8P3K2 | Yes |
| | campestris | dehydratase | | |
| Pp.FucD | Pseudomonas | L-fuconate | Q88J18 | No |
| | putida | dehydratase | | |
| Bj.TarD | Bradyrhizobium | D-tartrate | Q89FH0 | No |
| | japonicum | dehydratase | | |
| Aa.AraD | Acidovorax | D-arabinonate | F0Q4R8 | No |
| | avenae | dehydratase | | |
| Hh.AraD | Herbaspirillum | D-arabinonate | A0A4P7ADP2 | No |
| | huttiense | dehydratase | | |
| Hh.AraDC434S | Herbaspirillum | D-arabinonate | A0A4P7ADP2 | No |
| | huttiense | dehydratase | mutated in | |
| | | | position | |
| | | | C434S | |
| Ca.AraD | Clostridium | D-arabinonate | Q97L66 | No |
| | acetobutylicum | dehydratase | | |
| Pm.AraD | Paraburkholderia | D-arabinonate | NCBI acc | No |
| | mimosarum | dehydratase | no. | |
| | | | WP_028214996.1 | |
| Ec.UxaA | Escherichia coli | D-altronate | P42604 | No |
| | | dehydratase | | |

Table 3 also states whether codon-optimization was carried out.

In the following, the cloning, expression and purification of candidate enzymes are described:

The corresponding coding genes were amplified by PCR and cloned into the corresponding sites of the expression vector pET28a (company Novagen) using the cloning techniques and primer pairs listed in Table 4, wherein an N-terminal hexa-His tag was attached to the target sequence. The resulting plasmids were transformed into competent E. coli DH5a cells (NEB). The correct insertion was verified by isolating the plasmids and DNA sequencing before the plasmids thus obtained were transformed into the expression strain E. coli BL21 (DE3) (NEB). Depending on their suitability, the proteins were expressed in 50 mL LB medium or in auto induction medium (Studier F. W./2005/Prot Expr Purif/41/207-234/Protein production by auto-induction in high density shaking cultures)). After a sufficient incubation time, the cells were separated from the medium by centrifugation, which took place at 1700×g and 4° C. for 15 minutes. The cell pellets thus obtained were stored at −20° C. until further processing. The enzymes were purified by taking up the cell pellets in 1 mL of HEPES buffer (50 mM, pH 7) and subsequent ultrasound treatment in four intervals of 20 s each (UDS 751, Topas GmbH, output 40%). Cell debris was separated by centrifugation for 15 minutes at 13000×g and 4° C. and the subsequent transfer of the clear supernatant into a new reaction vessel. The target proteins were purified from the crude protein extract thus obtained by affinity chromatography in accordance with the manufacturer's instructions for cobalt resin (talon). The purified enzymes were then characterized with respect to their activity on the natural substrate (positive control) and the target substrate. The protein purification was carried out starting from the frozen cell spheres.

TABLE 4
Primer sequences, techniques and restriction sites used for cloning
the target genes into the pET28 expression vector

| Target | Cloning | Flanking | | |
|---|---|---|---|---|
| gene | technique | RE | Primer sequences (5′-3′)^{a} | |
| Ec.fsaA | PCR | NdeI | | agatatCATATGGAACTGTATC |
| | Restriction | EcoRI | | TGGATACTTCAGAC (SEQ |
| | | | | ID No. 49) |
| | | | | Ec-FsaA_pET_fw |
| | | | | agatatGAATTCTTAAATCGAC |
| | | | | GTTCTGCCAAACGC (SEQ |
| | | | | ID No. 50) |
| | | | | Ec-FsaA_pET_rv |
| Ec.fsaA^{TA} | PCR | NdeI | | agatatCATATGGAACTGTATC |
| | Restriction | | | TGGATACTTCAGAC (SEQ |
| | | | | ID No. 49) |
| | | | | Ec-FsaA_pET_fw |
| | | EcoRI | | agatatGAATTCTTAAATCGAC |
| | | | | GTTCTGCCAAACGC (SEQ |
| | | | | ID No. 50) |
| | | | | Ec-FsaA_pET_rv |
| Ec.mdh5Q | PCR | NdeI | | agatatCATATGAAAGTCGCA |
| | Restriction | | | GTCCTCGGC (SEQ ID No. |
| | | | | 51) |
| | | EcoRI | | Ec-Mdh22_pET_fw |
| | | | | agatatGAATTCTTACTTATTAA |
| | | | | CGAACTCTTCGCCCAG |
| | | | | (SEQ ID No. 52) |
| | | | | Ec-Mdh22_pET_rv |
| Tt.lac11 | HiFi | NdeI | 470 | ctggtgccgcgcggcagcCATATG |
| (N-tag) | | | | CGCAAACTGCTTGGC (SEQ |
| | | | | ID No. 53) |
| | | BamHI | 471 | gtcgacggagctcgaattcGGATCC |
| | | | | TTAGAACCCCAGTCCTTTG |
| | | | | G |
| | | | | (SEQ ID No. 54) |
| Tt.lac11 | HiFi | NcoI^{b} | 570 | actttaagaaggagatataCCATGC |
| (C-tag) | | | | GCAAACTGCTTGG (SEQ ID |
| | | | | No. 55) |
| | | HindIII | 572 | ggtgctcgagtgcggccgcAAGCTT |
| | | | | GAACCCCAGTCCTTTGGTTT |
| | | | | TC |
| | | | | (SEQ ID No. 56) |
| Tt.lac11v | HiFi | NcoI | 571 | actttaagaaggagatataCCATGG |
| 1 (C-tag) | | | | AACCGAGTCAGAATCC |
| | | | | (SEQ ID No. 57) |
| | | HindIII | 572 | ggtgctcgagtgcggccgcAAGCTT |
| | | | | GAACCCCAGTCCTTTGGTTT |
| | | | | TC |
| | | | | (SEQ ID No. 56) |
| Tt.lac11v | HiFi | NcoI | 573 | actttaagaaggagatataCCATGG |
| 2(C-tag) | | | | AACGCGCAGATC (SEQ ID |
| | | | | No. 57) |
| | | | | ggtgctcgagtgcggccgcAAGCTT |
| | | HindIII | 572 | GAACCCCAGTCCTTTGGTTT TC |
| | | | | (SEQ ID No. 56) |
| Tt.lac11v | HiFi | NcoI^{b} | 574 | actttaagaaggagatataCCATGT |
| 3 (C-tag) | | | | GGAGCGAAGGTCC (SEQ ID |
| | | | | No. 58) |
| | | HindIII | 572 | ggtgctcgagtgcggccgcAAGCTT |
| | | | | GAACCCCAGTCCTTTGGTTTTC |
| | | | | (SEQ ID No. 56) |
| Sc.ara1 | HiFi | NdeI | 153 | CAGCCATATGTCTTCTTCAG |
| | | | | TAGCCTCAACCGAAAAC (SEQ ID No. 60) |
| | | BamHI | 154 | gaattcGGATCCTTAATACTTT |
| | | | | AAATTGTCCAAGTTTGG (SEQ ID No. 61) |
| Sc.ara2 | HiFi | NdeI | 155 | cagcCATATGGTTAATGAAAA |
| | | | | AGTGAATCCATTCGAC |
| | | | | CTGGATGAGGAATACC |
| | | | | (SEQ ID No. 62) |
| | | EcoRI | 156 | agctcGAATTCTTATATCATTT |
| | | | | (SEQ ID No. 63) |
| Pc.tadH | HiFi | NdeI | 145 | catcacagcagcggcctggtgccgcgc |
| | | | | ggcagcCATATGTCTACCGAT |
| | | | | AGTTTACAACAGTTTC |
| | | | | (SEQ ID No. 64) |
| | | EcoRI | 146 | tgcggccgcaagcttgtcgacggagctc |
| | | | | GAATTCTTATGCCGGAACC |
| | | | | GGTGCAC (SEQ ID No. 65) |
| PI.lgdA | HiFi | NdeI | 147 | catcacagcagcggcctggtgccgcgc |
| | | | | ggcagcCATATGAGTAATGCC |
| | | | | GAAAAAGCAC (SEQ ID No. |
| | | | | (66) |
| | | EcoRI | 148 | tgcggccgcaagcttgtcgacggagctc |
| | | | | GAATTCTTAAAAATTCACCG |
| | | | | GCTG |
| | | | | (SEQ ID No. 67) |
| Ps.fdh | HiFi | NdeI | 149 | catcacagcagcggcctggtgccgcgc |
| | | | | ggcagcCATATGTCTTCTACT |
| | | | | GAACCTGC (SEQ ID No. 68) |
| | | EcoRI | 150 | tgcggccgcaagcttgtcgacggagctc |
| | | | | GAATTCTTACGGGGTCGGA |
| | | | | ATTAAG |
| | | | | (SEQ ID No. 69) |
| Ss.adh4 | HiFi | NdeI | 151 | catcacagcagcggcctggtgccgcgc |
| | | | | ggcagcCATATGGAGAACGTG |
| | | | | AATATGGTG (SEQ ID No. 70) |
| | | EcoRI | 152 | tgcggccgcaagcttgtcgacggagctc |
| | | | | GAATTCTTACGGGGTGATAA |
| | | | | CTTG |
| | | | | (SEQ ID No. 71) |
| Bm.fdh | PCR | NdeI | 468 | gcaggagcCATATGGATCTGA |
| | Restriction | | | ATCTGCAGGACAAGGTCGT |
| | | | | (SEQ ID No. 72) |
| | | HindIII | 469 | gcaggagcAAGCTTTCAGACG |
| | | | | AGCGCACGATCGAGATGCG |
| | | | | TAT |
| | | | | (SEQ ID No. 73) |
| Ec.ilvD | PCR | NheI | | agatatGCTAGCATGCCTAAGT |
| | Restriction | | | ACCGTTCCGCC |
| | | | | (SEQ ID No. 74) |
| | | | | Ec-IlvD_pET_fw |
| | | EcoRI | | agatatGAATTCTTAACCCCCC |
| | | | | AGTTTCGATTTATCG |
| | | | | (SEQ ID No. 75) |
| | | | | Ec-IlvD_pET_rv |
| Ss.ilvD | HiFi | NdeI | 180 | catcacagcagcggcctggtgccgcgc |
| | | | | ggcagcCATATGCCGGCAAAA |
| | | | | TTAAA |
| | | | | (SEQ ID No. 76) |
| | | EcoRI | 181 | tgcggccgcaagcttgtcgacggagctc |
| | | | | GAATTCTTAAGCGGGACGG |
| | | | | (SEQ ID No. 77) |
| Xc.fucD | HiFi | NdeI | 182 | catcacagcagcggcctggtgccgcgc |
| | | | | ggcagcCATATGCGTACCATT |
| | | | | ATCGC |
| | | | | (SEQ ID No. 78) |
| | | EcoRI | 183 | tgcggccgcaagcttgtcgacggagctc |
| | | | | GAATTCTTAGGCTTTGGCTT |
| | | | | (SEQ ID No. 79) |
| Pp.fucD | HiFi | NdeI | 486 | ctggtgccgcgcggcagcCATATGA |
| | | | | ACAGTGCCCCCGAC (SEQ ID No. 80) |
| | | BamHI | 487 | gtcgacggagctcgaattcGGATCC |
| | | | | TCAGGAACGGTTGATGTCG |
| | | | | (SEQ ID No. 81) |
| Bj.tarD | HiFi | NdeI | 339 | ctggtgccgcgcggcagcCATATGT |
| | | | | CCGTCCGCATCGTC |
| | | | | (SEQ ID No. 82) |
| | | BamHI | 340 | gtcgacggagctcgaattcGGATCC |
| | | | | TTACTCCGCCAGCGCCTT |
| | | | | (SEQ ID No. 83) |
| Aa.araD | HiFi | NdeI | 498 | ctggtgccgcgcggcagcCATATGT |
| | | | | CGACTGATGCACTGGC |
| | | | | (SEQ ID No. 84) |
| | | | | cggagctcgaattcGGATCCTCAC |
| | | BamHI | 499 | ATCACCGCGCCGAG |
| | | | | (SEQ ID No. 85) |
| Hh.araD | HiFi | NdeI | 500 | ctggtgccgcgcggcagcCATATGA |
| | | | | AAGCCAACTCTCCCG |
| | | | | (SEQ ID No. 86) |
| | | BamHI | 501 | cggagctcgaattcGGATCCTCA |
| | | | | GGTGTAGACGCCGATG |
| | | | | (SEQ ID No. 87) |
| Ec.uxaA | HiFi | NdeI | 482 | ctggtgccgcgcggcagcCATATG |
| | | | | CAATACATCAAGATCCATGC |
| | | | | (SEQ ID No. 88) |
| | | BamHI | 483 | gtcgacggagctcgaattcGGATCC |
| | | | | TTATAGCGTTACGCCGCTTT |
| | | | | TG |
| | | | | (SEQ ID No. 89) |

^{a}restriction sites are underlined in bold and the codon start/stop sequences are marked
^{b}restriction sites are lost after the cloning process

The identification of enzymes with D-threose dehydrogenase activity was carried out as follows:

In order to identify the D-threose dehydrogenase activity, the enzymes D-threo-aldose-1-dehydrogenase from Paraburkholderia caryophylli (Pc.TadH), D-arabinose dehydrogenases from Saccharomyces cerevisiae, Sc.Ara1 and Sc.Ara2, Scyllo-inositol-2-dehydrogenase from Paracoccus laeviglucosivorans (PI.LgdA), D-threo-aldose-1-dehydrogenase from Pseudomonas sp. 1143 (Ps.Fdh), D-arabinose dehydrogenase from Sulfolobus solfataricus (Ss.Adh4) and L-fucose dehydrogenase from Burkholderia multivorans (Bm.BmulJ04919), referred to here as Bm.Fdh, were amplified from genomic DNA using the primers of genomic DNA listed in Table 4 or starting from synthetic genes (see Table 3). The cloning into the expression vector pET28a was carried out using the methods specified in Table 4. In Table 4, the restriction sites are underlined, and the coding start/stop sequences are marked in bold.

FIG. 2 schematically shows the results of the screening of the candidate enzymes for NAD(P)-dependent D-threose dehydrogenase activity in the form of a column diagram. For the screening, substrate concentrations of 10 mM co-factor and sugar were set. In the absence of activity, the enzymes in FIG. 2 were marked with an asterisk (*). The enzyme activities are represented on a logarithmic scale in the column diagram. The results are the mean value of at least two independent biological experiments. The error bars correspond to the standard deviation from the mean value. The exact values are represented in Table 5. Table 5 shows the specific activities of the N-His-tagged enzyme suitable as D-threose dehydrogenase, expressed in U per mg of the purified enzyme, which was measured with a fixed amount (10 mM) of the substrates, D-arabinose or D-threose, and of the co-factors, NAD⁺ or NADP⁺. The abbreviation “n.d.” means “not detected”.

TABLE 5
Dehydrogenase activity of candidate enzymes on D-arabinose andD-threose

| | D-arabinose dehydrogenase | | D-threose dehydrogenase | |
|---|---|---|---|---|
| | in U*mg⁻¹ | | in U*mg⁻¹ | |
| Enzymes | NAD⁺ | NADP⁺ | NAD⁺ | NADP⁺ |
| Sc.Ara1 | 0.02 (±0.006) | 0.52 (±0.18) | n.d. | 0.02 (±0.007) |
| Sc.Ara2 | 0.018 (±0.025) | 0.12 (±0.05) | n.d. | n.d. |
| Pl.LgdA | 2.54 (±0.17) | 0.48 (±0.18) | n.d. | n.d. |
| Pc.TadH | 4.65 (±0.20) | 0.382 (±0.240) | 0.27 (±0.02) | n.d. |
| Bm.Fdh | 0.05 (±0.01) | n.d. | 0.06 (±0.005) | n.d. |
| Ps.Fdh | n.d. | 0.84 (±0.43) | n.d. | n.d. |
| Ss.Adh4 | n.d. | 0.053 (±0.075) | n.d. | n.d. |

Of a total of seven candidate enzymes, Sc.Ara1, Pc.TadH and Bm.Fdh showed a measurable activity on D-threose with one of the co-factors NAD⁺ or NADP⁺, which is also evident from FIG. 2 and Table 5. Since Pc. TadH showed the highest specific activity at 0.27 U*mg⁻¹, further kinetic analyses were carried out with this enzyme. A km value of 26.63 mM was determined for the substrate D-threose. Since Pc. TadH had the highest activity on D-threose and had a clear expression in E. coli, this enzyme was preferred for the construction of the metabolic pathway for synthesis.

The identification of enzymes with D-threonate dehydratase activity was possible, as was shown, with the help of in vitro tests, the results of which are described below. While dehydratase enzymes with activity on L-threonate, as already mentioned, are sufficiently known, such enzyme activities have not been reported on the corresponding D-stereoisomer up to now. Candidate enzymes with known activities on sugar acids with a (2S,3R) configuration were therefore selected analogously to the strategy used for the demonstration of the D-threose dehydrogenase activities. The selection of the candidate enzymes included the L-fuconate dehydratases from Xanthomonas campestris (Xc.FucD) and Pseudomonas putida (Pp.FucD), D-arabinonate dehydratases from Acidovorax avenae (Aa.AraD) and Herbaspirillum huttiense (Hh.AraD), D-tartrate dehydratase from Bradyrhizobium japonicum (Bj.TarD) and D-altronate dehydratase from Escherichia coli (Ec.UxaA). In addition, D-hydroxy acid dehydratases from E. coli (Ec. IIvD) and Sulfolobus solfataricus (Ss. IIvD) were included in the investigations. The corresponding genes were cloned into the pET28a vector by means of the primers and techniques described in Tables 3 and 4. After expression and purification of the enzymes in accordance with the methods described above, they were tested for their activity on D-threonate and other sugar acids as described above.

FIG. 3 shows a column diagram with the results of the screening of the mentioned candidate enzymes for D-threonate dehydratase activity. All purified candidate enzymes were tested on D-threonate and the corresponding natural substrate using the mentioned semicarbazide test, which recognises 2-keto acids. The substrate concentrations were adjusted to 10 mM, except for Aa.AraD and Hh.AraD, where 1 mM natural substrate was used. In the absence of activity, the enzymes are marked with an asterisk (*). The enzyme activities are represented on a logarithmic scale in the column diagram. The exact values of the activities are represented in Table 6. Table 6 shows the specific activities of the tested N-His-tagged enzymes. The results are indicated as mean value (+/−standard deviation) of at least two biological replicas. The abbreviation “n.d.” means “not detected”.

TABLE 6
Activity of various dehydratases on D-
threonate and their natural substrates

| | | Activity | |
|---|---|---|---|
| | | towards | Activity |
| | | natural | against (D)- |
| | | substrate | Threonate |
| Enzyme | Natural substrate | (U mg⁻¹) | (U mg⁻¹) |
| Ec.IlvD | 2R-dihydroxyvalerate | 0.88 (±0.32) | n.d. |
| Ss.IlvD | 2R-dihydroxyvalerate | 0.01 (±0.004) | n.d. |
| Xc.FucD | L-fuconate | 4.03 (±1.07) | n.d. |
| Pp.FucD | L-fuconate | n.d. | n.d. |
| Bj.TarD | D-tartrate | 4.68 (±0.67) | n.d. |
| Aa.AraD | D-arabinonate | 0.30 (±0.06)^{a} | 0.18 (±0.08) |
| Hh.AraD | D-arabinonate | 0.58 (±0.04)^{a} | 0.30 (±0.02) |
| Ec.UxaA | D-altronate | 0.07 (±0.05)^{a, b} | n.d. |

^{a}Enzyme activity tested on the substrate at a final concentration of 1 mM.
^{b} Enzyme activity in the presence of FeSO4 (0.5 mM). No activity was observedin the presence of MgCl2 (10 mM) or MnSO4 (10 mM).

Among the enzymes tested, Hh.Arad and Aa.AraD showed a significant activity on D-threonate, wherein they had specific activities of 0.30 U mg⁻¹ and 0.18 U mg⁻¹, respectively, as shown in Table 6 and FIG. 3.

The construction of a variant of the threono-1,4-lactonase Tt.Lac11 from T. terrifontis with improved expression in E. coli is described below. The lactonase Tt.Lac11 from T. terrifontis could be expressed in E. coli only with great difficulty, which resulted in a low yield of purified enzyme for kinetic characterisation, and a low activity of this enzyme in the production strain could be expected. An analysis of the amino acid sequence of the lactonase identified an N-terminal signal sequence which could affect the export of the enzyme into the periplasm. In order to improve the cytosolic expression of Tt.Lac11, N-terminally truncated variants of this protein were prepared and tested for their expressibility in E. coli.

It could be shown that using a variant of the enzyme truncated by 38 amino acids (A1-38), an expression increased by a factor of 34 can be achieved, as shown in Table 7. This variant is referred to below as Tt.Lac11v1, while a variant truncated by 51 amino acids is referred to as Tt.Lac11v2 and a variant truncated by 76 amino acids is referred to as Tt.Lac11v3. Table 7 shows yields and activities of truncated variants of the poly-His-tagged Tt.Lac11-lactonase on expression with pET28 in E. coli BL21 (DE3). The results correspond to mean value and standard deviation from two independent biological replicates.

TABLE 7
Yields and activities of truncated variants of the poly-His-tagged
Tt.Lac11-lactonase on expression with pET28 in E. coli BL21(DE3)

| | | | Activity (U mg⁻¹) on | |
|---|---|---|---|---|
| | | Concentration | 4 mM substrate | |
| Position His- | Expressed | after purification | L-fucono-1,4- | D-threono-1,4- |
| Tag | protein | (mg*mL⁻¹) | lactone | lactone |
| N-term | wt | 0.142 | 1.21 (±0.22) | 1.49 (±0.23) |
| C-term | wt | 0.211 | 4.22 (±0.28) | 4.71 (±1.83) |
| C-term | Δ1-38 | 4.886 | 16.46 (±2.01) | 12.35 (±1.97) |
| C-term | Δ1-51 | 2.076 | 12.10 (±0.85) | 7.65 (±0.04) |
| C-term | Δ1-76 | 0.134 | n.d. | 0.21 |

The biosynthesis of DHB from glycolaldehyde was demonstrated by simultaneous expression of the entire metabolic pathway in a production strain. For this purpose, the starting strain E. coli TW64 (MG1655 ΔyqhD ΔaldA) was used in all experiments and transformed with plasmids which ensured the expression of the synthetic metabolic pathway either completely or in part. The cells were cultivated in 250 mL shaking flasks on mineral salt medium, which was complemented with 10% (v/v) LB medium, at 37° C. and 220 rpm in an incubator (Infors). IPTG (0.5 mM) was added after the cultures reached an OD600 value of approximately 0.6. Glycolaldehyde (20 mM) was added to the cultures when the OD600 value of the cultures was about 2.0. The incubation time was 48 hours. The results were represented as mean value (+/−standard deviation) of at least two biological replicas. The concentrations of DHB and of the metabolic intermediates and glycolaldehyde were determined on an HPLC system (K-2600, Knaur) which was equipped with a UV-Vis detector (HP 1047A, Hewlett-Packard, USA). The injection volume was 20 μL and the substances were separated on a Rezex RoA-organic acid H+ column equipped with a SecurityGuard cartridge (Phenomenex, USA) using 0.5 mM H2SO4 as a mobile phase at a flow rate of 0.5 mL/min. The determination of D-glucose, D-threose, glycolaldehyde, D-threonate and acetate was carried out at 35° C., while DHB and ethylene glycol were measured at 80° C. Since D-threonolactone and D-threonate cannot be dissolved with this method, the concentrations of these metabolites are given as pooled values for “D-threonate/lactone”.

The results of the experiments on a bioconversion of glycolaldehyde (GA) to DHBare represented in Table 8.

TABLE 8
Results of bioconversion of 20 mM glycolaldehyde to DHB

| | | | | D- | |
|---|---|---|---|---|---|
| | | | | threonate/ | |
| | | | D-threose | lactone | DHB |
| | | GA (mM) | (mM) | (mM) | produced |
| Strain | Plasmids | (consumed) | (produced) | (produced) | (mM) |
| TW293 | pACT3- | 14.02 (±2.33) | 0.20 (±0.09) | 2.69 (±0.18) | — |
| | Ec.fsaA^{TA}- | | | | |
| | Pc.tadH-Tt.lac11/ | | | | |
| | pEXT22- | | | | |
| | Ec.mdh^{5Q}- | | | | |
| | Hh.araD | | | | |
| TW304 | pACT3- | 12.77 (±6.41) | 1.59 (±0.19) | 2.39 (±1.59) | 0.08 |
| | Ec.fsaA^{TA}- | | | | |
| | Pc.tadH-Tt.lac11/ | | | | |
| | pEXT22- | | | | |
| | Ec.mdh^{5Q}- | | | | |
| | Hh.araD/ | | | | |
| | pEXT21-Re.kdgT | | | | |
| TW354 | pACT3- | 15.61 (±2.44) | 2.99 (±0.41) | 1.41 (±0.14) | 0.16 (±0.04) |
| | Ec.fsaA^{TA}- | | | | |
| | Pc.tadH- | | | | |
| | Tt.lac11^{v1}/ | | | | |
| | pEXT22- | | | | |
| | Ec.mdh^{5Q}- | | | | |
| | Hh.araD/ | | | | |
| | pEXT21-Re.kdgT | | | | |

In strain TW293 all enzyme activities were expressed which are necessary according to the proposed metabolic pathway in order to convert glycol aldehyde to DHB. Surprisingly, the intermediate stages D-threose and D-threonate/lactone could be detected after 48 hours of cultivation, but no DHB (Table 8).

Since the lactonase Tt-Lac11 has a signal sequence which could affect the transport of this enzyme into the periplasm, it was suspected that the ring cleavage of the lactone to threonate takes place in the periplasm and thus a re-import of the resulting D-threonate becomes necessary. In order to verify this hypothesis, in addition to all enzymes of the metabolic pathway, the D-threonate-importing permease (Re.kdgT) from Cupriavidus necator was expressed in strain TW304. In fact, 0.08 mM of DHB from glycolaldehyde could be produced with this strain. This provides the proof of the function of the proposed metabolic pathway according to FIG. 1.

In order to eliminate the need for export or import of metabolic intermediates, three truncated forms of TtLac11 were produced, from which sections of their N-terminal sequences of different lengths were removed (Table 7). The lactonase variant Tt.Lac11v1 (Δ1-38 aa) showed a 34-fold improved expression in E. coli and a 10-fold improved specific activity of D-threono-1,4-lactone. This construct was therefore selected in order to provide the required cytoplasmic lactonase activity in the synthesis pathway in this exemplary embodiment. The strain (TW354) which expressed the improved lactonase was able to accumulate 0.16 mM DHB.

Identification of Enzymes with D-Threose Dehydrogenase Activity with the Help of Whole Cell Biotransformation's of Glycolaldehyde to DHB

In the preceding exemplary embodiment, it was shown that it is possible with the help of the proposed metabolic pathway to convert glycol aldehyde to DHB in a whole cell biotransformation. The expression of alternative candidate enzymes for individual reaction steps in the described metabolic pathway and the simultaneous measurement of the resulting DHB concentration can therefore serve to identify additional or more suitable enzymes with the desired activities. According to this strategy, a strain was first constructed which does not express D-threose dehydrogenase, but otherwise contains the entire metabolic pathway including the threonate permease. In addition, the strains TW 354, TW444, TW445 and TW446 were constructed which additionally expressed various candidate enzymes for the D-threose dehydrogenase activity. The strains were cultivated as in the above-described exemplary embodiment and the concentrations of DHB and other intermediates were measured after incubation for 48 hours.

As expected, the strain E. coli TW559 without D-threose dehydrogenase was not able to convert the D-threose synthesized from glycolaldehyde to DHB or the metabolic products D-threonate/lactone, as the results represented in Table 9 show. If the candidate enzymes Pc. TadH, Xc.Fdh or Aa. TadH were additionally expressed, the production of DHB could be detected, which showed that these enzymes have a D-threose dehydrogenase activity. In contrast, no DHB could be measured on expression of the Ppi. TadH, which showed that this enzyme either has no D-threose dehydrogenase activity or cannot be expressed sufficiently in E. coli.

TABLE 9
Results of the bioconversion of 10 mM glycolaldehyde
to DHB depending on the varied candidate enzymes
for D-threose dehydrogenase activity

| | | Metabolite concentrations after 48 h [mM] | | | |
|---|---|---|---|---|---|
| | | | D- | | |
| | Varied | | threonate/ | | |
| Strain | threose DH | D-threose | lactone(*) | DHB | EG |
| TW559 | none | 2.5 | n.d. (n.d.) | n.d. | 0.65 |
| TW354 | Pc.TadH | 0.05 (1.46) | n.d. (1.34) | 0.45 | 0.45 |
| TW444 | Aa.TadH | 0.37 (1.27) | n.d (0.11) | 0.29 | 1.46 |
| TW445 | Xc.fdh | n.d. (0.75) | n.d (0.08) | 1.13 | 0.34 |
| TW446 | Ppi.TadH | 0.34 (2.13) | n.d (0.13) | n.d | 0.69 |

(*)The values shown in brackets correspond to the concentrations of these substances after 24 h

Identification of Enzymes with D-Threonate Dehydratase Activity with the Help of Whole Cell Biotransformations of Glycolaldehyde to DHB

Similar to the preceding example, the identification of D-threonate dehydratases is possible by quantifying the DHB formation on glycolaldehyde after exchange of the Hh.AraD with other candidate enzymes in the production strain. A further criterion for the identification of improved D-threonate dehydratases is the rate of the D-threonate degradation. In accordance with this strategy, the mutant Hh.AraD C434S, the Ca.AraD and the enzyme Pm.AraD were expressed instead of the previously used Hh.AraD in the production strains TW452, TW453 and TW454, respectively, and investigated with regard to their influence on the rates of threonate degradation and DHB formation. As can be seen from the results represented in Table 10, the enzyme Pm.AraD does not provide any D-threonatedehydratase activity or cannot be sufficiently expressed in E. coli, since no DHB production can be detected in the production strain when this enzyme is used. In contrast, both the mutants Hh.AraD C434S and Pm.AraD allow the production of DHB, which proves their D-threonate dehydratase activity. In addition, the results show that the mutant Hh.AraD C434S degrades D-threonate more rapidly, which leads to an increased production of DHB.

TABLE 10
Results of the bioconversion of 20 mM glycolaldehyde
to DHB depending on the varied candidate enzymes
for D-threonate dehydratase activity

| | | Metabolite concentrations after 48 h [mM] | | | |
|---|---|---|---|---|---|
| | | | D- | | |
| | Variated threonate | | threonate/ | | |
| Strain | dehydratase | D-threose | lactone | DHB | EG |
| TW445 | Hh.AraD | n.d. | 0.52 | 0.52 | 0.31 |
| TW452 | Hh.AraD C434S | n.d. | 0.35 | 0.87 | 0.23 |
| TW453 | Ca.AraD | n.d. | 5.39 | n.d. | 0.41 |
| TW454 | Pm.AraD | n.d. | 1.31 | 0.35 | 0.21 |

### Identification of a Suitable NAD-Dependent Ethylene Glycol Dehydrogenase

In order to achieve the conversion of ethylene glycol (EG) to DHB or threonine with the help of the described metabolic pathway, the metabolic pathway must be expanded by a reaction which allows the oxidation of ethylene glycol to glycol aldehyde. As already mentioned, several enzymes are known which catalyze a NAD-dependent oxidation of ethylene glycol to glycol aldehyde. In order to test which enzyme is most suitable for complementing the described metabolic pathway, a growth-dependent test system was employed in which the growth rate of the test strain depends on the in vivo activity of the ethylene glycol dehydrogenase. It is known that E. coli naturally does not express ethylene glycol dehydrogenase and therefore cannot grow ethylene glycol as the sole carbon source on the substrate. However, the expression of an ethylene glycol dehydrogenase permits the conversion of ethylene glycol to glycol aldehyde and thus a growth on this substrate. Therefore, the three candidate enzymes Go.Adh, Ec.FucO and Ec.FucO 16L: L7V were expressed with the help of a pEXT20 vector in the E. coli strains E. coli ΔyqhD and E. coli ΔyqhD ΔaldA. The constructs based on the double mutant E. coli ΔyqhD ΔaldA served as a control, since these strains cannot grow on ethylene glycol as a result of the deletion of the glycolaldehyde dehydrogenase AldA. The test strains were incubated on mineral salt medium which had the same composition as in the experiments described above. In this medium, only the glucose as sole carbon source was replaced by 100 mM ethylene glycol. The test strains were incubated in microtiter plates (250 μL medium per well) at a shaking frequency of 880 rpm and a temperature of 37° C. in a microtiter plate reader (Tecan). The growth rates were determined by regular measurement of the OD600. As is shown in FIG. 4, the ethylene glycol dehydrogenase Go.Adh enabled the fastest growth, followed by Ec. FucO/6L: L7V and Ec.FucO. For this reason, the Go.Adh was subsequently employed as ethylene glycol dehydrogenase for the conversion of ethylene glycol to DHB with the help of the described metabolic pathway.

Demonstration of the Synthesis of DHB Starting from Ethylene Glycol

The biosynthesis of DHB from ethylene glycol was demonstrated by the simultaneous expression of the entire metabolic pathway including the ethylene glycol dehydrogenase in a production strain. For this purpose, the starting strain E. coli TW64 (MG1655 ΔyqhD ΔaldA) was used in all experiments and transformed with plasmids which ensured the expression of the synthetic metabolic pathway including the ethylene glycol dehydrogenase Go.Adh. The cultivation of the production strain TW363 constructed in this way and the measurement of the substrate and product concentrations took place as in the above-mentioned exemplary embodiment for the synthesis of DHB from glycolaldehyde. In contrast to the example mentioned, no glycol aldehyde was added to the cultures, but ethylene glycol was added to the cultures in various initial concentrations after an OD of 0.6 was reached. The results of these experiments are represented in Table 11.

TABLE 11
Results of the bioconversion of ethylene glycol (EG) to DHB by strain TW363

| | | Products formed [mM] | | | |
|---|---|---|---|---|---|
| Initial | | | | D- | |
| concentration | Consumption of | Glycolal- | D- | threonate/ | |
| EG (mM) | EG [mM] | dehyde | threose | lactone | DHB |
| 0 | — | 0.00 | 0.06 | 0.00 | 0.00 |
| 160 | 7.20 | 0.00 | 0.05 | 0.00 | 0.00 |
| 320 | 10.3 | 0.42 | 0.05 | 0.17 | 0.36 |
| 640 | 17.0 | 3.87 | 0.29 | 1.57 | 0.19 |
| 1280 | 30.8 | 6.34 | 1.08 | 2.09 | 0.00 |

Construction of a OHB Reductase with a Higher Specifity for NADPH than for NADH

In order to construct an OHB reductase with a preference for the co-factor NADPH, amino acid exchanges were carried out individually or simultaneously in the already described NADH-dependent OHB reductase Ec.Mdh^{5Q} in positions D34 and I35. The construction of the template plasmid pET28-Ec.Mdh^{5Q} and the method used therefore was described in Frazão, C. J. R.; Topham, C. M.; Malbert, Y.; François, J. M.; Walther, T. Rational Engineering of a Malate Dehydrogenase for Microbial Production of 2,4-Dihydroxybutyric Acid via Homoserine Pathway. Biochem. J. 2018, 475 (23), 3887-3901. The additional mutations were introduced using the same method by inverse PCR with the primers listed in Table 12. The PCR products were digested with Dpnl to remove the template plasmid and transformed into E. coli DH5alpha cells. The resulting plasmids were isolated and the correctness of the DNA sequence was verified by sequencing.

TABLE 12
Used primers for the introduction of mutations into the template
enzyme Ec.Mdh^{5Q}

| Mutations | Primer sequences (5′-3′) | |
|---|---|---|
| D34G | 261 (fw_sdm_Ecmdh_D34G) | TCAGAACTCTCTCTGTATGGCAT |
| | | CGCTCCAGTGACTCCCGG |
| | | (SEQ ID NO. 90) |
| | 262 (rv_sdm_Ecmdh_D34G) | CCGGGAGTCACTGGAGCGATG |
| | | CCATACAGAGAGAGTTCTGA |
| | | (SEQ ID NO. 91) |
| 135S | 263 (fw_sdm_Ecmdh_i35s) | GAACTCTCTCTGTATGATTCTGC |
| | | TCCAGTGACTCCCGGTG |
| | | (SEQ ID NO. 92) |
| | 264 (rv_sdm_Ecmdh_i35s) | CACCGGGAGTCACTGGAGCAG |
| | | AATCATACAGAGAGAGTTC |
| | | (SEQ ID NO. 93) |
| D34G | 456 | GAACTCTCTCTGTATGGCTCTG |
| 135S | (fw_sdm_Ecmdh_D34G_i35s) | CTCCAGTGACTCCCGGTG |
| | | (SEQ ID NO. 94) |
| | 457 | CACCGGGAGTCACTGGAGCAG |
| | (rv_sdm_Ecmdh_D34G_i35s) | AGCCATACAGAGAGAGTTC |
| | | (SEQ ID NO. 95) |
| D34G | 558 | GAACTCTCTCTGTATGGCAAAG |
| 135K | (fw_sdm_Ecmdh_D34G_i35k) | CTCCAGTGACTCCCGGTG |
| | | (SEQ ID NO. 96) |
| | 559 | CACCGGGAGTCACTGGAGCTT |
| | (rv_sdm_Ecmdh_D34G_i35k) | TGCCATACAGAGAGAGTTC |
| | | (SEQ ID NO. 97) |
| D34Gv | 560 | GAACTCTCTCTGTATGGCCGTG |
| 135R | (fw_sdm_Ecmdh_D34G_i35r) | CTCCAGTGACTCCCGGTG |
| | | (SEQ ID NO. 98) |
| | 561 | CACCGGGAGTCACTGGAGCAC |
| | (rv_sdm_Ecmdh_D34G_i35r) | GGCCATACAGAGAGAGTTC |
| | | (SEQ ID NO. 99) |
| D34G | 562 | GAACTCTCTCTGTATGGCACCG |
| 135T | (fw_sdm_Ecmdh_D34G_i35T) | CTCCAGTGACTCCCGGTG (SEQ |
| | | ID NO. 100) |
| | 563 | CACCGGGAGTCACTGGAGCGG |
| | (rv_sdm_Ecmdh_D34G_i35T) | TGCCATACAGAGAGAGTTC |
| | | (SEQ ID NO. 101) |

The enzyme variants thus obtained were expressed in E. coli as described above, purified and characterized with respect to their kinetic parameters. It could be shown that the mutation D35G alone or in combination with mutations in position I35 shifts the co-factor specificity of the OHB reductase in the direction of NADPH, as shown in Table 13. The enzyme variant with the highest activity and specificity on NADPH (Ec.Mdh^{5Q} D35G:I35R) is referred to below as Ec-Mdh^{7Q}. The kinetic parameters of this enzyme were determined in detail and are listed in Table 14.

TABLE 13
Co-factor specificity of OHB reductase mutants

| | VNADH | VNADPH | VNADPH/ |
|---|---|---|---|
| Enzyme | [U mg⁻¹] | [U mg⁻¹] | VNADH |
| Ec.Mdh^{5Q} | 47.3 | 1.5 | 0.03 |
| Ec.Mdh^{5Q} D34G | 15.0 | 33.5 | 2.2 |
| Ec.Mdh^{5Q} I35S | 44.9 | 2.2 | 0.03 |
| Ec.Mdh^{5Q} D34G | 6.9 | 32.2 | 4.7 |
| I35K | | | |
| Ec.Mdh^{5Q} D34G | 7.5 | 50.8 | 6.7 |
| I35R | 3.6 | 18.5 | 5.2 |
| Ec.Mdh^{5Q} D34G | 6.1 | 18.9 | 3.1 |
| I35S | | | |
| Ec.Mdh^{5Q} D34G | | | |
| I35T | | | |

The specific activities were determined at constant initial concentrations of the substrates OHB (2 mM) and NAD(P)H (0.25 mM). The enzyme Ec.Mdh^{5Q} is the already described NADH-dependent OHB reductase Ec.Mdh I12V:R81A:M85Q:D86S:G179D.

It was shown that Ec.Mdh^{7Q} has a specificity for NADPH which is greater by a factor of 8600 than the starting enzyme Ec.Mdh^{5Q}.

TABLE 14
Analysis of the kinetic parameters of the OHB
reductase variants of Ec.Mdh^{5Q} and Ec.Mdh^{7Q}
(Ec.MdhI12V:R81A:M85Q:D86S:G179D:D34G:I35R)

| Substrate | Ec.Mdh^{5Q} | Ec.Mdh^{7Q} |
|---|---|---|
| NADH ^{a} | | |
| Km [mM] | 0.02 | 0.42 |
| Vmax [U mg⁻¹] | 81.3 | 21.0 |
| Vmax/Km [U mg⁻¹ mM⁻¹] | 4064 | 50 |
| NADPH ^{a} | | |
| Km [mM] | 0.51 | 0.10 |
| Vmax [U mg⁻¹] | 4.4 | 86.0 |
| Vmax/Km [U mg⁻¹ mM⁻¹] | 9 | 859 |
| OHB ^{b} | | |
| Km [mM] | 2.26 | 0.66 |
| Vmax [U mg⁻¹] | 103.1 | 73.5 |
| Vmax/Km [U mg⁻¹ mM⁻¹] | 46 | 111 |
| Co-factor specifity | | |
| (vmax/Km)NADPH/(vmax/Km) | 0.002 | 17.2 |
| NADH | | |
| Catalytic efficiency | | |
| (vmax/Km)OHB * | 186.944 | 95.349 |
| (vmax/Km)NAD(P)H | | |

The OHB reductase activity was determined at a constant OHB concentration (2 mM) and a varied NAD(P)H concentration (2-0.002 mM).

The OHB reductase activity was determined at a constant NAD(P)H concentration (0.25 mM) and a varied OHB concentration (10-0.05 mM).

The kinetic parameters Km and Vmax were determined by adapting the measured initial reaction rates to the Michaelis-Menten model with the help of Matlab.

### Increased DHB Production Through the Use of a NADPH-Dependent OHB Reductase

In the following, the suitability of the use of a NADPH-dependent OHB reductase for improving the DHB production was investigated. For this purpose, the strains TW354 and TW469, which differ only by the expression of the NADH- or NADPH-dependent OHB reductase, were cultivated under identical conditions (starting substrate 10 mM glycolaldehyde, further details on experimental and analytical conditions see above) and the DHB accumulation was compared after 48 hours. It could be shown that the use of a NADPH-dependent OHB reductase significantly improves the DHB production, as shown in Table 15.

TABLE 15
Results of the bioconversion of 10 mM glycolaldehyde
to DHB by E. coli strains expressing either
a NADH- or a NADPH-dependent OHB reductase

| | | Products formed after 48 h (in mM) | | | |
|---|---|---|---|---|---|
| Strain | Enzyme | D-threose | D-threonate | DHB | EG |
| TW354 | Ec.Mdh^{5Q} | 0.05 | n.d. | 0.45 | 0.45 |
| TW469 | Ec.Mdh^{7Q} | n.d. | n.d. | 1.34 | 0.44 |

n.d.—not detectable

Demonstration of the Synthesis of L-Threonine Starting from Glycolaldehyde (GA)

The biosynthesis of L-threonine from glycolaldehyde was achieved by the simultaneous expression of the entire metabolic pathway including the enzymes for conversion of OHB to threonine. For this purpose, the starting strain E. coli TW64 (MG1655 ΔyqhD ΔaldA) was used in all experiments and transformed with plasmids which ensured the expression of the synthetic metabolic pathway including the homoserine transaminase Ec.AspC. The resulting strain was named TW612. In order to further improve threonine production, threonine-exporting permease RhtB, homoserine kinase Ec. ThrB and threonine synthase Ec. ThrC were overexpressed in this strain. This was achieved by replacing the native promoter of the respective genes in the chromosome by the strong constitutive promoter proD Davis, J. H.; Rubin, A. J.; Sauer, R. T.: Design, construction and characterization of a set of insulated bacterial promoters. In: Nucleic Acid Res., 2011, 3, pp. 1131-1141. The strain thus obtained was named TW613.

Threonine can be synthesized in the production strain both via the synthetic metabolic pathway and via the natural metabolic pathway. In order to demonstrate unequivocally that threonine has been synthesized via the synthetic metabolic pathway according to the invention, on the one hand, a control experiment was carried out using the strain TW619 which expresses only an incomplete and therefore non-functional variant of the synthetic metabolic pathway. In particular, this strain contained no genetic information for the expression of a threonate dehydratase. Furthermore, in the experiments, completely ¹³C-labeled glycol aldehyde (Omicron Biochemicals) was employed as a substrate, and the proportion of labeled and unlabeled threonine in the culture medium was compared with one another. By detecting completely labeled threonine, it can be demonstrated that the corresponding carbon is derived from glycolaldehyde.

The stem cultures were carried out at 37° C. on a rotary shaker (Infors HT, Germany) at 220 rpm. The precultures were incubated in 5 mL LB in 50 mL Falcon tubes. After about 10 hours, 500 μL of these cultures were used for inoculating a second preculture (10 mL of 90% v/v M9 mineral medium and 10% v/v LB in 50 mL Falcon tubes) which was cultivated overnight. The biomass required for the production of main cultures with a starting OD600 of 0.2 was transferred into a medium of 90% (v/v) mineral M9 medium and 10% (v/v) LB. Antibiotics were added to all media in standard concentrations (chloramphenicol, 35 μg mL-1; kanamycin, 50 μg mL-1; spectinomycin, 100 μg mL-1). IPTG (0.5 mM) was added when the OD600 reached ˜0.6. After reaching an OD600 of 2, completely ¹³C-labeled glycol aldehyde was added. The samples for the analysis of the extracellular metabolites were taken on a regular basis. A 1 mL culture sample was centrifuged (at 13,000 g for 5 min.) and the supernatant was stored at −20° C. until further use. The samples were filtered with 0.2 μm PTFE membrane syringe filters before the measurement.

LC/MS analysis: The cell-free supernatant was diluted 100 times in a solution of 10 mM ammonium acetate (pH 9.2), which was dissolved in 60% (v/v) acetonitrile and 40% (v/v) water. The LC-MS platform consists of a Vanquish and a Thermo Scientific™ Q Exactive™ Focus (all from ThermoFisher Scientific, San Jose, CA) controlled by the Xcalibur 2.1 software. Separation by liquid chromatography was carried out with a SeQuant® ZIC® PHILIC (5 μm polymer 150×2.1 mm) column with a flow rate of 0.15 mL min-1. A gradient of A (5% ACN, 10 mM ammonium acetate, pH 9.2 by NH₄OH) and B (90% ACN, 10 mM ammonium acetate, pH 9.2 by NH₄OH) was used for optimum separation efficiency. The gradient was 0 min, 95% B; 2 min, 95% B; 3 min, 89.4% B; 5 min, 89.4% B; 6 min, 83.8% B; 7 min, 83.8% B; 8 min, 78.2% B; 9 min, 78.2% B; 10 min, 55.9% B; 12 min, 55.9% B; 13 min, 27.9% B; 16 min., 27.9% B; 18 min., 0% B; 23 min., 0% B; 24 min., 95% B; 30 min., 95% B. The temperature of the sample taker was kept at 6° C., the injection volume was 5 μL and the oven temperature was kept at 25° C. The device settings for the electrospray ionization were optimized for a flow rate of 0.15 mL min-1. Further parameters were set as follows: Flow rate of the mantle gas 32 (device-specific units), flow rate of the auxiliary gas 8 (device-specific units), flow rate of the sweep gas 0 (device-specific units), spray voltage-3.5 kV, capillary temperature 250° C. and auxiliary gas temperature 200° C.

The results of these experiments are represented in FIG. 5, which include column diagrams with the results of a ¹³C-based metabolic material flow analysis, which shows the biosynthesis of L-threonine from glycolaldehyde (GA) via the synthetic metabolic pathway. Only unlabeled threonine (M+0) or fully labeled threonine (M+4) was found in the culture medium.

The control strain TW619 produced only small amounts of threonine. In addition, no labeled threonine was detectable in the culture medium of this strain, as a result of which it was shown that threonine in this strain was prepared exclusively from glucose. In contrast, completely labeled threonine was found in the culture medium of the strains TW612 and TW613 which express the entire synthetic metabolic pathway. Through these experiments, it was possible to demonstrate without doubt that the synthetic metabolic pathway is suitable for the production of threonine.

Table 16 lists the SEQ ID numbers of the DNA sequences of genes encoding certain enzymes and the SEQ ID numbers of the amino acid sequences of the corresponding enzymes, respectively.

TABLE 16
Sequences for enzymes

| Enzyme | DNA sequence | Amino acid sequence |
|---|---|---|
| Ec.FucO | SEQ ID No. 102 | SEQ ID No. 103 |
| Ec.FucO^{OR} | SEQ ID No. 104 | SEQ ID No. 105 |
| (Ec.FucOI6L:L7V) | | |
| Go.Adh | SEQ ID No. 106 (codon-optimized) | SEQ ID No. 107 |
| Ec.FsaA | SEQ ID No. 108 | SEQ ID No. 109 |
| Ec.FsaA^{TA} | SEQ ID No. 110 | SEQ ID No. 111 |
| Sc.Ara1 | SEQ ID No. 112 | SEQ ID No. 113 |
| Sc.Ara2 | SEQ ID No. 114 | SEQ ID No. 115 |
| Pc.TadH | SEQ ID No. 116 (codon-optimized) | SEQ ID No. 117 |
| Pl.LgdA | SEQ ID No. 118 (codon-optimized) | SEQ ID No. 119 |
| Ps.Fdh | SEQ ID No. 120 (codon-optimized) | SEQ ID No. 121 |
| Xc.Fdh | SEQ ID No. 122 | SEQ ID No. 123 |
| Aa.TadH | SEQ ID No. 124 | SEQ ID No. 125 |
| Ppi.TadH | SEQ ID No. 126 | SEQ ID No. 127 |
| Ss.Adh4 | SEQ ID No. 128 (codon-optimized) | SEQ ID No. 129 |
| Bm.Fdh | SEQ ID No. 130 | SEQ ID No. 131 |
| Tt.lac11 | SEQ ID No. 132 (codon-optimized) | SEQ ID No. 133 |
| Tt.lac11^{v1} | SEQ ID No. 134 (codon-optimized) | SEQ ID No. 135 |
| Tt.lac11^{v2} | SEQ ID NO. 136 (codon-optimized) | SEQ ID No. 137 |
| Tt.lac11^{v3} | SEQ ID No. 138 (codon-optimized) | SEQ ID No. 139 |
| Ec.IlvD | SEQ ID No. 140 | SEQ ID No. 141 |
| Ss.IlvD | SEQ ID No. 142 (codon-optimized) | SEQ ID No. 143 |
| Xc.FucD | SEQ ID No. 144 (codon-optimized) | SEQ ID No. 145 |
| Pp.FucD | SEQ ID No. 146 | SEQ ID No. 147 |
| Bj.TarD | SEQ ID No. 148 | SEQ ID No. 149 |
| Aa.AraD | SEQ ID No. 150 | SEQ ID No. 151 |
| Hh.AraD | SEQ ID No. 152 | SEQ ID No. 153 |
| Hh.AraDC434S | SEQ ID No. 154 | SEQ ID No. 155 |
| Ca.AraD | SEQ ID No. 156 | SEQ ID No. 157 |
| Pm.AraD | SEQ ID No. 158 | SEQ ID No. 159 |
| Ec.UxaA | SEQ ID No. 160 | SEQ ID No. 161 |
| Ec.Mdh^{5Q} | SEQ ID No. 162 | SEQ ID No. 163 |
| Re.KdgT | SEQ ID No. 164 | SEQ ID No. 165 |
| Ec.AspC | SEQ ID No. 166 | SEQ ID No. 167 |

Table 17 lists the SEQ ID numbers of the DNA sequences for different plasmids.

TABLE 17
DNA sequences for plasmids

| Plasmid | DNA sequence |
|---|---|
| pACT3-Ec.fsaA^{TA}-Pc.tadH-Tt.lac11^{v1} | SEQ ID No. 168 |
| pACT3-Ec.fsaA^{TA}-Xc.fdh-Tt.lac11^{v1} | SEQ ID No. 169 |
| pACT3-Go.adh-Ec.fsaA^{TA}-Pc.tadH-Tt.lac11^{v1} | SEQ ID No. 170 |
| pEXT22-Ec.mdh^{5Q}-Hh.araD | SEQ ID No. 171 |

Table 18 lists the SEQ ID numbers of the DNA sequences of genes encoding different OHB reductase mutants and the SEQ ID numbers of the amino acid sequences of the corresponding OHB reductase mutants, respectively.

TABLE 18
Sequences for OHB reductase mutants

| Enzyme | DNA sequence | Amino acid sequence |
|---|---|---|
| Ec.Mdh^{5Q} | SEQ ID No. 162 | SEQ ID No. 163 |
| Ec.Mdh^{5Q} D34G | SEQ ID No. 172 | SEQ ID No. 173 |
| Ec.Mdh^{5Q} I35S | SEQ ID No. 174 | SEQ ID No. 175 |
| Ec.Mdh^{5Q} D34G I35K | SEQ ID No. 176 | SEQ ID No. 177 |
| Ec.Mdh^{5Q} D34G | SEQ ID No. 178 | SEQ ID No. 179 |
| I35R(Ec.Mdh^{7Q}) | | |
| Ec.Mdh^{5Q} D34G I35S | SEQ ID No. 180 | SEQ ID No. 181 |
| Ec.Mdh^{5Q} D34G I35T | SEQ ID No. 182 | SEQ ID No. 183 |

The DNA sequence for the plasmid pEXT22-Ec.mdh^{7Q}-Hh.araD is assigned the SEQ ID No. 184.

Sequence listing - free text:

| SEQ ID No. | Free text |
|---|---|
| 1-48: | primer sequence for plasmid construction |
| 49-89: | primer sequence for cloning a target gene into thepET28 |
| | expression vector |
| 90-101: | primer sequence for introducing mutations into the |
| | Ec.Mdh^{5Q} enzyme |
| 104: | sequence for mutant of Ec.FucO |
| 105: | mutant of Ec.FucO |
| 106: | codon-optimized sequence for Go.Adh |
| 110: | sequence for mutant of Ec.FsaA |
| 111: | mutant of Ec.FsaA |
| 116: | codon-optimized sequence for Pc.TadH |
| 118: | codon-optimized sequence for Pl.LgdA |
| 120: | codon-optimized sequence for Ps.Fdh |
| 126: | codon-optimized sequence for Ppi.TadH |
| 128: | codon-optimized sequence for Ss.Adh4 |
| 132: | codon-optimized sequence for Tt.lac11 |
| 134, 136, 138: | codon-optimized sequence for truncated variant ofTt.lac11 |
| 135: | variant of Tt.Lac11 truncated by 38 amino acids |
| 137: | variant of Tt.Lac11 truncated by 51 amino acids |
| 139: | variant of Tt.Lac11 truncated by 76 amino acids |
| 142: | codon-optimized sequence for Ss.IlvD |
| 144: | codon-optimized sequence for Xc.FucD |
| 154: | sequence for mutant of Hh.araD |
| 155: | mutant of Hh.araD with mutation of cysteine to serine |
| | at position 434 |
| 162, 172, 174, 176, 178, | |
| 180, 182: | sequence for mutant of Ec.Mdh |
| 163: | mutant of Ec.Mdh with mutations I12V R81A M85Q |
| | D86S G179D |
| 168, 169, 170, 171, 184: | sequence for plasmid |
| 173: | mutant of Ec.Mdh with mutations I12V R81A M85Q |
| | D86S G179D D34G |
| 175: | mutant of Ec.Mdh with mutations I12V R81A M85Q |
| | D86S G179D I35S |
| 177: | mutant of Ec.Mdh with mutations I12V R81A M85Q |
| | D86S G179D D34G I35K |
| 179: | mutant of Ec.Mdh with mutations I12V R81A M85Q |
| 181: | D86S G179D D34G I35R |
| 183: | mutant of Ec.Mdh with mutations I12V R81A M85Q |
| | D86S G179D D34G I35S |
| | mutant of Ec.Mdh with mutations I12V R81A M85Q |
| | D86S G179D D34G I35T |

### LIST OF USED REFERENCE NUMERALS AND ABBREVIATIONS

I xylose isomerase
II xylulose-1-kinase
III xylulose-1P-aldolase
IV Ethylene glycol dehydrogenase (membrane-bound)
V Ethylene glycol dehydrogenase (cytosolic)
VI methanol dehydrogenase
VII glycolaldehyde synthase
VIII D-threose aldolase
IX D-threose dehydrogenase
X D-threono-1,4-lactonase
XI D-threonate dehydratase
XII L-homoserine transaminase
XIII L-homoserine kinase
XIV L-threonine synthase
XV 2-keto-4-hydroxybutyrate (OHB) reductase
ATCC American Type Culture Collection
DH Dehydrogenase
DHB 2,4-dihydroxybutyrate, 2,4-dihydroxybutyric acid
EG Ethylene glycol
HEPES 2-(4-(2-hydroxyethyl)-1-piperazinyl)-ethanesulfonic acid
HMTB D/L-2-hydroxy-4-(methylthio) butyrate
IPTG isopropyl-3-D-thiogalactopyranoside
LB Lysogeny broth (LB) medium, complex nutrient medium for the cultivation of bacteria
NAD nicotinamide adenine dinucleotide
NADH protonated or reduced form of nicotinamide adenine dinucleotide
NADP nicotinamide adenine dinucleotide phosphate
NADPH protonated or reduced form of nicotinamide adenine dinucleotide phosphate
OD Optical density
OD600 Optical density at a wavelength of 600 nm
OHB 2-keto-4-hydroxybutyrate in the form of a 2-keto-4-hydroxybutyrate salt or of the acid 2-keto-4-hydroxybutyric acid
PCR Polymerase chain reaction
HiFi high-fidelity polymerase
RBS ribosome binding sequence
RE Restriction enzymes
UniProt. bioinformatic database for proteins of all living organisms and viruses (English: universal protein database)

1. A method for producing 2,4-dihydroxy butyrate (DHB) or L-threonine using a microbial metabolic pathway, comprising the following steps:
enzymatic conversion of glycolaldehyde to threose using a threose-aldolase,
enzymatic conversion of threose to threono-1,4-lactone using a threose dehydrogenase,
enzymatic conversion of threono-1,4-lactone to threonate using a threono-1,4-lactonase and
enzymatic conversion of threonate to 2-keto-4-hydroxybutyrate (OHB) using a threonate-dehydratase, and further comprising,
enzymatic conversion of 2-keto-4-hydroxybutyrate (OHB) to 2,4-dihydroxy butyrate (DHB) using a OHB reductase, or enzymatically converting 2-keto-4-hydroxybutyrate to L-homoserine using an L-homoserine transaminase, followed by a step of enzymatically converting L-homoserine to O-phospho-L-homoserine using a homoserine kinase using under ATP consumption, and
a step of enzymatically converting O-phospho-homoserine to L-threonine using an L-threonine synthase,
wherein the metabolic pathway is expressed in a microbial production strain which was previously modified from a wild type form into the microbial production strain by introducing at least one gene of such genes as are necessary for expression of the said enzymes into the production strain.

2. The method according to claim 1, expression of the genes is achieved by using plasmids or by integration of genes in the genome.

3. The method according to claim 1, wherein the production strain already has one or more enzymes required for the metabolic pathway in the wild type form.

4. The method according to claim 3, wherein a modified strain of the species Escherichia coli or the species Pseudomonas putida is used as a production strain.

5. The method according to claim 4, wherein a strain of the species Escherichia coli used as the production strain which has deletions in the genes coding for the aldehyde dehydrogenase (AldA) and/or the glycol aldehyde reductase (YqhD).

6. The method according to claim 5, wherein the genetic information the expressing enzyme D-threo-aldose-1-dehydrogenase from at least one of Paraburkholderia caryophylli (Pc.TadH) and Xanthomonas campestris (Xc.Fdh) or a genetic information expressing the enzyme D-arabinose dehydrogenase from Saccharomyces cerevisiae (Sc.Ara1) or from Acidovorax avenae (Aa.TadH) or genetic information expressing the enzyme L-fucose dehydrogenase from Burkholderia multivorans (Bm.Fdh) is introduced into a genome of the production strain.

7. The method according to claim 6, wherein for expression of D-threonate dehydratase in the production strain, the genetic information expressing the enzyme D-arabinonate dehydratase from Acidovorax avenae (Aa-AraD) and/or Herbaspirillum huttiense (Hh-AraD) and/or Paraburkholderia mimosarum (Pm.AraD) and/or that of the optimized mutant Hh.AraD C434S is introduced into the genome of the production strain.

8. The method according to claim 5 wherein for the expression of the D-threose aldolase in the production strain, the genetic information expressing the enzyme D-fructose-6-phosphate aldolase from Escherichia coli (Ec.FsaA) and/or that of the mutated variant Ec.FsaA L107Y: A129G (Ec.FsaA^{TA}) is introduced into the genome of the production strain.

9. The method according to claim 5 wherein for the expression of the threono-1,4-lactonase in the production strain, the genetic information expressing the enzyme gluconolactonase from Thermogutta terrifontis (Tt.Lac11) and/or that of a truncated variant of this enzyme (Tt.Lac11v1) is introduced into the genome of the production strain.

10. The method according to claim 5 wherein a threonate-importing enzyme is expressed in the production strain in addition to enzymes of the metabolic pathway.

11. The method according to claim 10, wherein the D-threonate-importing permease from Cupriavidus necator (Re.kdgT) is expressed in the production strain.

12. The method according to claim 11, further comprising at least one preceding step of microbially producing glycol aldehyde from ethylene glycol, methanol or xylose.

13. The method according to claim 12, wherein an OHB reductase which has a higher specificity for NADPH compared to NADH is used for the conversion of 2-keto-4-hydroxybutyrate (OHB) to 2,4-dihydroxybutyrate (DHB).

14. The method according to claim 13, wherein for the expression of the NADPH-preferring OHB reductase in the production strain, the genetic information expressing a mutated variant of the enzyme L-malate dehydrogenase from Escherichia coli (Ec.Mdh) is introduced into the genome of the production strain, wherein the mutated enzyme has a further mutation in at least one of positions D34 and I35 in addition to five point mutations I12V, R81A, M85Q, D86S and G179D as compared to the wild type enzyme.

15. The method according to claim 14, wherein for expression of the NADPH-preferring OHB reductase in the production strain, genetic information expressing one of the enzymes of the group consisting of Ec.Mdh I12V:R81A:M85Q:D86S:G179D:D34G,
Ec.Mdh I12V:R81A:M85Q:D86S:G179D:I35S,
Ec.Mdh I12V:R81A:M85Q:D86S:G179D:D34G:I35K,
Ec.Mdh I12V:R81A:M85Q:D86S:G179D:D34G:I35R (Ec.Mdh^{7Q}),
Ec.Mdh I12V:R81A:M85Q:D86S:G179D:D34G:I35S
and Ec.Mdh I12V:R81A:M85Q:D86S:G179D:D34G:I35T is introduced into the genome of the production strain.

16. An enzyme with 2-keto-4-hydroxybutyrate (OHB) reductase activity which catalyzes a conversion of 2-keto-4-hydroxybutyrate (OHB) to 2,4-dihydroxybutyrate (DHB), said enzyme being a mutant of the L-malatedehydrogenase from Escherichia coli (Ec.Mdh), wherein the mutated enzyme has a further mutation in at least one of positions D34 and I35 in addition to five point mutations I12V, R81A, M85Q, D86S and G179D as compared to the wild type enzyme.

17. The enzyme according to claim 15, wherein the enzyme is selected from the group consisting of the following enzymes:
Ec.Mdh I12V:R81A:M85Q:D86S:G179D:D34G,
Ec.Mdh I12V:R81A:M85Q:D86S:G179D: 13
Ec.Mdh I12V:R81A:M85Q:D86S:G179D:D34G:I35K,
Ec.Mdh I12V:R81A:M85Q:D86S:G179D:D34G:I35R (Ec.Mdh^{7Q}),
Ec.Mdh/12V:R81A:M85Q:D86S:G179D:D34G:I35S and
Ec.Mdh/12V:R81A:M85Q:D86S:G179D:D34G:I35T.

18. A method of using an enzyme according to claim 17 for a conversion of OHB to 2,4-DHB.


SEQUENCE LISTING (184 sequences)

SEQ ID NO: 1 - DNA, 60 bp, Artificial Sequence
  Primer sequence for Plasmid Construction
taagcactcgaggtttaactttaagaaggagatataccatggctaacagaatgctggtga

SEQ ID NO: 2 - DNA, 34 bp, Artificial Sequence
  Primer sequenz for Plasmid Construction
tgcttatctagattaccaggcggtatggtaaagc

SEQ ID NO: 3 - DNA, 60 bp, Artificial Sequence
  Primer sequence for Plasmid Construction
taagcactcgaggtttaactttaagaaggagatataccatggctaacagaatgattctga

SEQ ID NO: 4 - DNA, 29 bp, Artificial Sequence
  Primer sequence for Plasmid Construction
tgcttatctagattaggaccggaagtcga

SEQ ID NO: 5 - DNA, 56 bp, Artificial Sequence
  Primer sequence for Plasmid Construction
taagcactcgaggtttaactttaagaaggagatataccatggctgatacaatgctc

SEQ ID NO: 6 - DNA, 31 bp, Artificial Sequence
  Primer sequence for Plasmid Construction
taagcactcgagtgtgtgaaattgttatccg

SEQ ID NO: 7 - DNA, 16 bp, Artificial Sequence
  Primer sequence for Plasmid Construction
ttcgagctcggtaccc

SEQ ID NO: 8 - DNA, 63 bp, Artificial Sequence
  Primer sequence for Plasmid Construction
taagcaggatccgtttaactttaagaaggagatataccatggaactgtatctggatacttcag

SEQ ID NO: 9 - DNA, 34 bp, Artificial Sequence
  Primer sequence for Plasmid Construction
tgcttatctagattaaatcgacgttctgccaaac

SEQ ID NO: 10 - DNA, 36 bp, Artificial Sequence
  Primer sequence for Plasmid Construction
tgcttaatttaaatttaaatcgacgttctgccaaac

SEQ ID NO: 11 - DNA, 64 bp, Artificial Sequence
  Primer sequence for Plasmid Construction
taagcaatttaaatgtttaactttaagaaggagatataccatgtctaccgatagtttacaacag

SEQ ID NO: 12 - DNA, 29 bp, Artificial Sequence
  Primer sequence for Plasmid Construction
tgcttatctagattatgccggaaccggtg

SEQ ID NO: 13 - DNA, 56 bp, Artificial Sequence
  Primer sequence for Plasmid Construction
taagcaggtaccgtttaactttaagaaggagatataccatggctgatacaatgctc

SEQ ID NO: 14 - DNA, 30 bp, Artificial Sequence
  Primer sequence for Plasmid Construction
tgcttaggatccttaggaccggaagtcgag

SEQ ID NO: 15 - DNA, 58 bp, Artificial Sequence
  Primer sequence for Plasmid Construction
taagcaggtaccgtttaactttaagaaggagatataccatgcgcaaactgcttggcag

SEQ ID NO: 16 - DNA, 38 bp, Artificial Sequence
  Primer sequence for Plasmid Construction
tgcttagtcgacttagaaccccagtcctttggttttcg

SEQ ID NO: 17 - DNA, 56 bp, Artificial Sequence
  Primer sequence for Plasmid Construction
taagcagagctcgtttaactttaagaaggagatataccatgaaagtcgcagtcctc

SEQ ID NO: 18 - DNA, 35 bp, Artificial Sequence
  Primer sequence for Plasmid Construction
tgcttaggatccttacttattaacgaactcttcgc

SEQ ID NO: 19 - DNA, 58 bp, Artificial Sequence
  Primer sequence for Plasmid Construction
taagcaggatccgtttaactttaagaaggagatataccatgtcgactgatgcactggc

SEQ ID NO: 20 - DNA, 30 bp, Artificial Sequence
  Primer sequence for Plasmid Construction
tgcttatctagatcacatcaccgcgccgag

SEQ ID NO: 21 - DNA, 57 bp, Artificial Sequence
  Primer sequence for Plasmid Construction
taagcaggatccgtttaactttaagaaggagatataccatgaaagccaactctcccg

SEQ ID NO: 22 - DNA, 31 bp, Artificial Sequence
  Primer sequence for Plasmid Construction
tgcttatctagatcaggtgtagacgccgatg

SEQ ID NO: 23 - DNA, 63 bp, Artificial Sequence
  Primer sequence for Plasmid Construction
taagcaggatccgtttaactttaagaaggagatataccatgcagatttctatcaaacgcgcca

SEQ ID NO: 24 - DNA, 29 bp, Artificial Sequence
  Primer sequence for Plasmid Construction
tgcttaaagctttcatgcggcggtcctca

SEQ ID NO: 25 - DNA, 62 bp, Artificial Sequence
  Primer sequence for Plasmid Construction
taagcaatttaaatgtttaactttaagaaggagatataccatgaaggtcacagaaacacgcc

SEQ ID NO: 26 - DNA, 28 bp, Artificial Sequence
  Primer sequence for Plasmid Construction
tgcttatctagatcatggcgcggctccg

SEQ ID NO: 27 - DNA, 68 bp, Artificial Sequence
  Primer sequence for Plasmid Construction
taagcaatttaaatgtttaactttaagaaggagatataccatgaatacacgtcgccaattcctgtctg

SEQ ID NO: 28 - DNA, 30 bp, Artificial Sequence
  Primer sequence for Plasmid Construction
tgcttatctagatcatccagccgccggcac

SEQ ID NO: 29 - DNA, 57 bp, Artificial Sequence
  Primer sequence for Plasmid Construction
taagcaatttaaatgtttaactttaagaaggagatataccatgaatcggcgcacagg

SEQ ID NO: 30 - DNA, 30 bp, Artificial Sequence
  Primer sequence for Plasmid Construction
tgcttatctagactaaacgggctcgtgtgt

SEQ ID NO: 31 - DNA, 33 bp, Artificial Sequence
  Primer sequence for Plasmid Construction
ggttctgcctatggcagcgcaccggcaccgtcg

SEQ ID NO: 32 - DNA, 33 bp, Artificial Sequence
  Primer sequence for Plasmid Construction
cgacggtgccggtgcgctgccataggcagaacc

SEQ ID NO: 33 - DNA, 75 bp, Artificial Sequence
  Primer sequence for Plasmid Construction
taagcaggatccgtttaactttaagaaggagatataccatgaaaaatgttataaagataaatgaaaaagataatg

SEQ ID NO: 34 - DNA, 48 bp, Artificial Sequence
  Primer sequence for Plasmid Construction
tgcttatctagattatagtgttacaccgtttttaaatatagatatttc

SEQ ID NO: 35 - DNA, 59 bp, Artificial Sequence
  Primer sequence for Plasmid Construction
taagcaggatccgtttaactttaagaaggagatataccatgaagacctcaacagcagac

SEQ ID NO: 36 - DNA, 31 bp, Artificial Sequence
  Primer sequence for Plasmid Construction
tgcttatctagatcaggtgatcgcgccgatc

SEQ ID NO: 37 - DNA, 70 bp, Artificial Sequence
  Primer sequence for Plasmid Construction
tgtctttgctgatctgctacgtaccctctcatggaagttaggagtctgacgtgtaggctggagctgcttc

SEQ ID NO: 38 - DNA, 47 bp, Artificial Sequence
  Primer sequence for Plasmid Construction
gatagggacgacgtggtgttagctgtgcatatgaatatcctccttag

SEQ ID NO: 39 - DNA, 20 bp, Artificial Sequence
  Primer sequence for Plasmid Construction
cacagctaacaccacgtcgt

SEQ ID NO: 40 - DNA, 86 bp, Artificial Sequence
  Primer sequence for Plasmid Construction
aacccgacgctcatattggcactggaagccggggcataaactttaaccatataatacctcctaaagttaaacaaaattatttgtag

SEQ ID NO: 41 - DNA, 20 bp, Artificial Sequence
  Primer sequence for Plasmid Construction
attgccgaagtggatggtaa

SEQ ID NO: 42 - DNA, 20 bp, Artificial Sequence
  Primer sequence for Plasmid Construction
gtgactacatctccgagcaa

SEQ ID NO: 43 - DNA, 70 bp, Artificial Sequence
  Primer sequence for Plasmid Construction
tgcgacagtagcgtattgtggcacaaaaatagacacaccgggagttcatcgtgtaggctggagctgcttc

SEQ ID NO: 44 - DNA, 86 bp, Artificial Sequence
  Primer sequence for Plasmid Construction
cttaaaatgatcgatgtcagcaggtaggcaaaccaccattctaaggtcatataatacctcctaaagttaaacaaaattatttgtag

SEQ ID NO: 45 - DNA, 23 bp, Artificial Sequence
  Primer sequence for Plasmid Construction
catggtaaaagcagcaaacgcgt

SEQ ID NO: 46 - DNA, 23 bp, Artificial Sequence
  Primer sequence for Plasmid Construction
tatgaatcgccagtccggtctga

SEQ ID NO: 47 - DNA, 58 bp, Artificial Sequence
  Primer sequence for Plasmid Construction
taagcagagctcgtttaactttaagaaggagatataccatgtttgagaacattaccgc

SEQ ID NO: 48 - DNA, 31 bp, Artificial Sequence
  Primer sequence for Plasmid Construction
tgcttaggatccttacagcactgccacaatc

SEQ ID NO: 49 - DNA, 36 bp, Artificial Sequence
  Primer sequence for cloning of a target gene into the pET28-expression vector
agatatcatatggaactgtatctggatacttcagac

SEQ ID NO: 50 - DNA, 36 bp, Artificial Sequence
  Primer sequence for cloning of a target gene into the pET28-expression vector
agatatgaattcttaaatcgacgttctgccaaacgc

SEQ ID NO: 51 - DNA, 30 bp, Artificial Sequence
  Primer sequence for cloning of a target gene into the pET28-expression vector
agatatcatatgaaagtcgcagtcctcggc

SEQ ID NO: 52 - DNA, 39 bp, Artificial Sequence
  Primer sequence for cloning of a target gene into the pET28-expression vector
agatatgaattcttacttattaacgaactcttcgcccag

SEQ ID NO: 53 - DNA, 39 bp, Artificial Sequence
  Primer sequence for cloning of a target gene into the pET28-expression vector
ctggtgccgcgcggcagccatatgcgcaaactgcttggc

SEQ ID NO: 54 - DNA, 45 bp, Artificial Sequence
  Primer sequence for cloning of a target gene into the pET28-expression vector
gtcgacggagctcgaattcggatccttagaaccccagtcctttgg

SEQ ID NO: 55 - DNA, 38 bp, Artificial Sequence
  Primer sequence for cloning of a target gene into the pET28-expression vector
actttaagaaggagatataccatgcgcaaactgcttgg

SEQ ID NO: 56 - DNA, 47 bp, Artificial Sequence
  Primer sequence for cloning of a target gene into the pET28-expression vector
ggtgctcgagtgcggccgcaagcttgaaccccagtcctttggttttc

SEQ ID NO: 57 - DNA, 41 bp, Artificial Sequence
  Primer sequence for cloning of a target gene into the pET28-expression vector
actttaagaaggagatataccatggaaccgagtcagaatcc

SEQ ID NO: 58 - DNA, 37 bp, Artificial Sequence
  Primer sequence for cloning of a target gene into the pET28-expression vector
actttaagaaggagatataccatggaacgcgcagatc

SEQ ID NO: 59 - DNA, 38 bp, Artificial Sequence
  Primer sequence for cloning of a target gene into the pET28-expression vector
actttaagaaggagatataccatgtggagcgaaggtcc

SEQ ID NO: 60 - DNA, 37 bp, Artificial Sequence
  Primer sequence for cloning of a target gene into the pET28-expression vector
cagccatatgtcttcttcagtagcctcaaccgaaaac

SEQ ID NO: 61 - DNA, 39 bp, Artificial Sequence
  Primer sequence for cloning of a target gene into the pET28-expression vector
gaattcggatccttaatactttaaattgtccaagtttgg

SEQ ID NO: 62 - DNA, 37 bp, Artificial Sequence
  Primer sequence for cloning of a target gene into the pET28-expression vector
cagccatatggttaatgaaaaagtgaatccattcgac

SEQ ID NO: 63 - DNA, 38 bp, Artificial Sequence
  Primer sequence for cloning of a target gene into the pET28-expression vector
agctcgaattcttatatcatttctggatgaggaatacc

SEQ ID NO: 64 - DNA, 64 bp, Artificial Sequence
  Primer sequence for cloning of a target gene into the pET28-expression vector
catcacagcagcggcctggtgccgcgcggcagccatatgtctaccgatagtttacaacagtttc

SEQ ID NO: 65 - DNA, 54 bp, Artificial Sequence
  Primer sequence for cloning of a target gene into the pET28-expression vector
tgcggccgcaagcttgtcgacggagctcgaattcttatgccggaaccggtgcac

SEQ ID NO: 66 - DNA, 58 bp, Artificial Sequence
  Primer sequence for cloning of a target gene into the pET28-expression vector
catcacagcagcggcctggtgccgcgcggcagccatatgagtaatgccgaaaaagcac

SEQ ID NO: 67 - DNA, 52 bp, Artificial Sequence
  Primer sequence for cloning of a target gene into the pET28-expression vector
tgcggccgcaagcttgtcgacggagctcgaattcttaaaaattcaccggctg

SEQ ID NO: 68 - DNA, 56 bp, Artificial Sequence
  Primer sequence for cloning of a target gene into the pET28-expression vector
catcacagcagcggcctggtgccgcgcggcagccatatgtcttctactgaacctgc

SEQ ID NO: 69 - DNA, 53 bp, Artificial Sequence
  Primer sequence for cloning of a target gene into the pET28-expression vector
tgcggccgcaagcttgtcgacggagctcgaattcttacggggtcggaattaag

SEQ ID NO: 70 - DNA, 57 bp, Artificial Sequence
  Primer sequence for cloning of a target gene into the pET28-expression vector
catcacagcagcggcctggtgccgcgcggcagccatatggagaacgtgaatatggtg

SEQ ID NO: 71 - DNA, 52 bp, Artificial Sequence
  Primer sequence for cloning of a target gene into the pET28-expression vector
tgcggccgcaagcttgtcgacggagctcgaattcttacggggtgataacttg

SEQ ID NO: 72 - DNA, 40 bp, Artificial Sequence
  Primer sequence for cloning of a target gene into the pET28-expression vector
gcaggagccatatggatctgaatctgcaggacaaggtcgt

SEQ ID NO: 73 - DNA, 43 bp, Artificial Sequence
  Primer sequence for cloning of a target gene into the pET28-expression vector
gcaggagcaagctttcagacgagcgcacgatcgagatgcgtat

SEQ ID NO: 74 - DNA, 33 bp, Artificial Sequence
  Primer sequence for cloning of a target gene into the pET28-expression vector
agatatgctagcatgcctaagtaccgttccgcc

SEQ ID NO: 75 - DNA, 37 bp, Artificial Sequence
  Primer sequence for cloning of a target gene into the pET28-expression vector
agatatgaattcttaaccccccagtttcgatttatcg

SEQ ID NO: 76 - DNA, 53 bp, Artificial Sequence
  Primer sequence for cloning of a target gene into the pET28-expression vector
catcacagcagcggcctggtgccgcgcggcagccatatgccggcaaaattaaa

SEQ ID NO: 77 - DNA, 47 bp, Artificial Sequence
  Primer sequence for cloning of a target gene into the pET28-expression vector
tgcggccgcaagcttgtcgacggagctcgaattcttaagcgggacgg

SEQ ID NO: 78 - DNA, 53 bp, Artificial Sequence
  Primer sequence for cloning of a target gene into the pET28-expression vector
catcacagcagcggcctggtgccgcgcggcagccatatgcgtaccattatcgc

SEQ ID NO: 79 - DNA, 48 bp, Artificial Sequence
  Primer sequence for cloning of a target gene into the pET28-expression vector
tgcggccgcaagcttgtcgacggagctcgaattcttaggctttggctt

SEQ ID NO: 80 - DNA, 39 bp, Artificial Sequence
  Primer sequence for cloning of a target gene into the pET28-expression vector
ctggtgccgcgcggcagccatatgaacagtgcccccgac

SEQ ID NO: 81 - DNA, 44 bp, Artificial Sequence
  Primer sequence for cloning of a target gene into the pET28-expression vector
gtcgacggagctcgaattcggatcctcaggaacggttgatgtcg

SEQ ID NO: 82 - DNA, 39 bp, Artificial Sequence
  Primer sequence for cloning of a target gene into the pET28-expression vector
ctggtgccgcgcggcagccatatgtccgtccgcatcgtc

SEQ ID NO: 83 - DNA, 43 bp, Artificial Sequence
  Primer sequence for cloning of a target gene into the pET28-expression vector
gtcgacggagctcgaattcggatccttactccgccagcgcctt

SEQ ID NO: 84 - DNA, 41 bp, Artificial Sequence
  Primer sequence for cloning of a target gene into the pET28-expression vector
ctggtgccgcgcggcagccatatgtcgactgatgcactggc

SEQ ID NO: 85 - DNA, 38 bp, Artificial Sequence
  Primer sequence for cloning of a target gene into the pET28-expression vector
cggagctcgaattcggatcctcacatcaccgcgccgag

SEQ ID NO: 86 - DNA, 40 bp, Artificial Sequence
  Primer sequence for cloning of a target gene into the pET28-expression vector
ctggtgccgcgcggcagccatatgaaagccaactctcccg

SEQ ID NO: 87 - DNA, 39 bp, Artificial Sequence
  Primer sequence for cloning of a target gene into the pET28-expression vector
cggagctcgaattcggatcctcaggtgtagacgccgatg

SEQ ID NO: 88 - DNA, 44 bp, Artificial Sequence
  Primer sequence for cloning of a target gene into the pET28-expression vector
ctggtgccgcgcggcagccatatgcaatacatcaagatccatgc

SEQ ID NO: 89 - DNA, 47 bp, Artificial Sequence
  Primer sequence for cloning of a target gene into the pET28-expression vector
gtcgacggagctcgaattcggatccttatagcgttacgccgcttttg

SEQ ID NO: 90 - DNA, 41 bp, Artificial Sequence
  Primer sequence for introducing mutations into the Ec.Mdh5Q enzyme
tcagaactctctctgtatggcatcgctccagtgactcccgg

SEQ ID NO: 91 - DNA, 41 bp, Artificial Sequence
  Primer sequence for introducing mutations into the Ec.Mdh5Q enzyme
ccgggagtcactggagcgatgccatacagagagagttctga

SEQ ID NO: 92 - DNA, 40 bp, Artificial Sequence
  Primer sequence for introducing mutations into the Ec.Mdh5Q enzyme
gaactctctctgtatgattctgctccagtgactcccggtg

SEQ ID NO: 93 - DNA, 40 bp, Artificial Sequence
  Primer sequence for introducing mutations into the Ec.Mdh5Q enzyme
caccgggagtcactggagcagaatcatacagagagagttc

SEQ ID NO: 94 - DNA, 40 bp, Artificial Sequence
  Primer sequence for introducing mutations into the Ec.Mdh5Q enzyme
gaactctctctgtatggctctgctccagtgactcccggtg

SEQ ID NO: 95 - DNA, 40 bp, Artificial Sequence
  Primer sequence for introducing mutations into the Ec.Mdh5Q enzyme
caccgggagtcactggagcagagccatacagagagagttc

SEQ ID NO: 96 - DNA, 40 bp, Artificial Sequence
  Primer sequence for introducing mutations into the Ec.Mdh5Q enzyme
gaactctctctgtatggcaaagctccagtgactcccggtg

SEQ ID NO: 97 - DNA, 40 bp, Artificial Sequence
  Primer sequence for introducing mutations into the Ec.Mdh5Q enzyme
caccgggagtcactggagctttgccatacagagagagttc

SEQ ID NO: 98 - DNA, 40 bp, Artificial Sequence
  Primer sequence for introducing mutations into the Ec.Mdh5Q enzyme
gaactctctctgtatggccgtgctccagtgactcccggtg

SEQ ID NO: 99 - DNA, 40 bp, Artificial Sequence
  Primer sequence for introducing mutations into the Ec.Mdh5Q enzyme
caccgggagtcactggagcacggccatacagagagagttc

SEQ ID NO: 100 - DNA, 40 bp, Artificial Sequence
  Primer sequence for introducing mutations into the Ec.Mdh5Q enzyme
gaactctctctgtatggcaccgctccagtgactcccggtg

SEQ ID NO: 101 - DNA, 40 bp, Artificial Sequence
  Primer sequence for introducing mutations into the Ec.Mdh5Q enzyme
caccgggagtcactggagcggtgccatacagagagagttc

SEQ ID NO: 102 - DNA, 1149 bp, Escherichia coli
atggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 103 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 104 - DNA, 1149 bp, Artificial Sequence
  Sequence for mutant of Ec.FucO
atggctaacagaatgctggtgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 105 - protein, 382 aa, Artificial Sequence
  Mutant of Ec.FucO
MANRMLVNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 106 - DNA, 1032 bp, Artificial Sequence
  Codon-optimised sequence for Go.Adh
atggctgatacaatgctcgccgccgtcgtccgtgaattcggcaagccgctctccatcgagcggctacccatcccggacatcaagccccaccagatcctcgtgaaggtcgatacctgtggcgtctgccacactgacctgcacgccgcgcgcggggactggccgtccaagcccaacccgccgttcattcccgggcatgaaggcgtcggacacatcgtcgccgtcggcagtcaggtcggcgatttcgtcaagaccggcgatgtcgtgggcgtgccctggctctactccgcctgcggtcactgcgaacactgtctgggcggctgggaaacactctgcgaaaagcaggacgacaccggctacaccgtcaatggctgcttcgccgaatatgtcgtggcagacccgaactacgtcgcacacctgccctcgaccatcgacccgcttcaggcctcgccggtcctgtgcgcggggctgacggtctataagggcctgaaaatgacggaggcccgccccggccagtgggtcgcagtctcgggcgtcggcggtctcggccagatggccgtgcagtacgccgtcgccatgggcatgaatgtcgtcgcggtggacatcgatgacgaaaaactcgccacagccaaaaagctcggcgcatccctgaccgtcaacgccaaggacacggacccggccaggttcatccagcagcagatcggcggcgcacatggcgctctcgtcaccgctgtcggacggacggcgttttcgcaggccatgggctatgcccgccgcggcggcaccatcgtcctgaacggactgccgcccggcgatttcccggtctcgatcttcgacatggtcatgaacggcaccaccatccgtggctccatcgtcggaacacggctggacatgatcgaggccatggatttcttcgcccgcggcaaggtcaaatccgtcgtcacccccggaaaacttgaaaacatcaatacgatcttcgacgatctgcagaatggtcgcctcgaaggccggacagtgctcgacttccggtcctaa

SEQ ID NO: 107 - protein, 343 aa, Gluconobacter oxydans
MADTMLAAVVREFGKPLSIERLPIPDIKPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPARFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 108 - DNA, 663 bp, Escherichia coli
atggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgctgggaaccgcggtatatggcgcagcacaagggctgctgtcggcgctggcaggtgcggaatatgttgcgccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaa

SEQ ID NO: 109 - protein, 220 aa, Escherichia coli
MELYLDTSDVVAVKALSRIFPLAGVTTNPSIIAAGKKPLDVVLPQLHEAMGGQGRLFAQVMATTAEGMVNDALKLRSIIADIVVKVPVTAEGLAAIKMLKAEGIPTLGTAVYGAAQGLLSALAGAEYVAPYVNRIDAQGGSGIQTVTDLHQLLKMHAPQAKVLAASFKTPRQALDCLLAGCESITLPLDVAQQMISYPAVDAAVAKFEQDWQGAFGRTSI

SEQ ID NO: 110 - DNA, 663 bp, Artificial Sequence
  Sequence for mutant of Ec.FsaA
atggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgtatggaaccgcggtatatggcgcagcacaagggctgctgtcggcgctggcaggtgcggaatatgttggcccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaa

SEQ ID NO: 111 - protein, 220 aa, Artificial Sequence
  Mutant of Ec.FsaA
MELYLDTSDVVAVKALSRIFPLAGVTTNPSIIAAGKKPLDVVLPQLHEAMGGQGRLFAQVMATTAEGMVNDALKLRSIIADIVVKVPVTAEGLAAIKMLKAEGIPTYGTAVYGAAQGLLSALAGAEYVGPYVNRIDAQGGSGIQTVTDLHQLLKMHAPQAKVLAASFKTPRQALDCLLAGCESITLPLDVAQQMISYPAVDAAVAKFEQDWQGAFGRTSI

SEQ ID NO: 112 - DNA, 1035 bp, Saccharomyces cerevisiae
atgtcttcttcagtagcctcaaccgaaaacatagtcgaaaatatgttgcatccaaagactacagaaatatacttttcactcaacaatggtgttcgtatcccagcactgggtttggggacagcaaatcctcacgaaaagttagctgaaacaaaacaagccgtaaaagctgcaatcaaagctggatacaggcacattgatactgcttgggcctacgagacagagccattcgtaggtgaagccatcaaggagttattagaagatggatctatcaaaagggaggatcttttcataaccacaaaagtgtggccggttctatgggacgaagtggacagatcattgaatgaatctttgaaagctttaggcttggaatacgtcgacttgctcttgcaacattggccgctatgttttgaaaagattaaggaccctaaggggatcagcggactggtgaagactccggttgatgattctggaaaaacaatgtatgctgccgacggtgactatttagaaacttacaagcaattggaaaaaatttaccttgatcctaacgatcatcgtgtgagagccattggtgtctcaaatttttccattgagtatttggaacgtctcattaaggaatgcagagttaagccaacggtgaaccaagtggaaactcaccctcacttaccacaaatggaactaagaaagttctgctttatgcacgacattctgttaacagcatactcaccattaggttcccatggcgcaccaaacttgaaaatcccactagtgaaaaagcttgccgaaaagtacaatgtcacaggaaatgacttgctaatttcttaccatattagacaaggcactatcgtaattccgagatccttgaatccagttaggatttcctcgagtattgaattcgcatctttgacaaaggatgaattacaagagttgaacgacttcggtgaaaaatacccagtgagattcatcgatgagccatttgcagccatccttccagagtttactggtaacggaccaaacttggacaatttaaagtattaa

SEQ ID NO: 113 - protein, 344 aa, Saccharomyces cerevisiae
MSSSVASTENIVENMLHPKTTEIYFSLNNGVRIPALGLGTANPHEKLAETKQAVKAAIKAGYRHIDTAWAYETEPFVGEAIKELLEDGSIKREDLFITTKVWPVLWDEVDRSLNESLKALGLEYVDLLLQHWPLCFEKIKDPKGISGLVKTPVDDSGKTMYAADGDYLETYKQLEKIYLDPNDHRVRAIGVSNFSIEYLERLIKECRVKPTVNQVETHPHLPQMELRKFCFMHDILLTAYSPLGSHGAPNLKIPLVKKLAEKYNVTGNDLLISYHIRQGTIVIPRSLNPVRISSSIEFASLTKDELQELNDFGEKYPVRFIDEPFAAILPEFTGNGPNLDNLKY

SEQ ID NO: 114 - DNA, 1008 bp, Saccharomyces cerevisiae
atggttaatgaaaaagtgaatccattcgacttagcttcggtttctcctttagtcttaggaggtgcaatattgaaccagcaatatacagatgagccagaatccattccactggaagacataatcaaatacgcattttcgcatggtatcaatgcaattgacacttctccctactatggccctagtgaggttctttatggtagggcactgtccaatttaaggaatgaatttcccagagacacttattttatatgcacaaaggttgggcgtattggtgcggaagagtttaactattctagagatttcgtgagatttagtgttcacagatcgtgcgaaagattacacactacatatctcgatttggtgtatttgcacgatgtcgagtttgtcaaatttccagacatcttagaggcattaaaggaattgaggaccttgaagaacaaaggagttatcaaaaattttggcatatcaggctacccaatcgactttatcacctggcttgctgagtactgctctaccgaggaaagtgatattgggtcattggatgcagtattgtcctattgtaacttgaacttacaaaacaataagctattgaactttcgagaaagactgctacgcaatgctaaactgaaaatggtttgcaacgcatcaatcttgagcatgtcgctactaaggtcacaggagacaagacagtttcacccgtgttctcatgaattgagagaatgcgcatctcaggctgccaaatactgtcaagaacaaaatgtcgacttagccgatttggccacaagatacgcgatttcggaatgggtaggaaagggacccgttgttcttggagtcagtagcatggaagaattgaaacttgctctggataattacgaaatagtgaaatcgaatggcaacagattatcttctaaggacggacaactagtagaatatatccaaaagaatatttttaaagaacattttaatgaagagtggtcttcaggtattcctcatccagaaatgatataa

SEQ ID NO: 115 - protein, 335 aa, Saccharomyces cerevisiae
MVNEKVNPFDLASVSPLVLGGAILNQQYTDEPESIPLEDIIKYAFSHGINAIDTSPYYGPSEVLYGRALSNLRNEFPRDTYFICTKVGRIGAEEFNYSRDFVRFSVHRSCERLHTTYLDLVYLHDVEFVKFPDILEALKELRTLKNKGVIKNFGISGYPIDFITWLAEYCSTEESDIGSLDAVLSYCNLNLQNNKLLNFRERLLRNAKLKMVCNASILSMSLLRSQETRQFHPCSHELRECASQAAKYCQEQNVDLADLATRYAISEWVGKGPVVLGVSSMEELKLALDNYEIVKSNGNRLSSKDGQLVEYIQKNIFKEHFNEEWSSGIPHPEMI

SEQ ID NO: 116 - DNA, 1005 bp, Artificial Sequence
  Codon-optimised sequence for Pc.TadH
atgtctaccgatagtttacaacagtttcgtcgccgtgtggcaattccgggtccggctggttttggtgcagcaccgctgggcaatctgtttagccgtgttaccgatgaggccgcacaagaaactttaagtgccgcatggggtgctggtatccgctactttgataccgcaccgttctatggcatgggtctgagtgaacgccgtctgggtcgcgcactggccagctatccgcgtgatgccttcgtgattagcaccaaagtgggtcgtttactgatcgccgacgacagtgtgccggaaacccagcacggttatgttggtggtttaccgtaccgcgttgaatacgactatactgccgatggcgcccgccgtagcattgaaagctctttagaacgtttaggtttagatcgtattgacattgtgtatattcacgacgtggccgaggatacccatggtccgctgtggcgtcagcattatcgtaccgccgctggtggtgcaatgaaagctttaagccgtttacgcgatgaaggtgtgattggtggctggggtttaggcgtgaatcgtgttgaaccgtgtctgatggctttagccgatgccgatccggacgtctttttaatcgccggccgctatactttactggataccacagctttagatgctttaattccggcatgcgaagcacgcggtgcacgtttagttgttggtggtccgtacaatagtggtttactggctggtggcgatacctttgaatatgcccgcgcagatgccgcaatgttagcccgtcgcgcacgtttattaagccactgcgaacgctttggtgttgatttaaaggccgccgctttacagttttgtaaagccccgggcgttgttgcatgcgttattgccggtgcccgcaatgcagatgaggttcgtcagaactgcgcagcaatgacagcaccggtgccgcgcgaattttggcaagctttaaaggaagagggcttagttccgcaaggtgcaccggttccggcataa

SEQ ID NO: 117 - protein, 334 aa, Paraburkholderia caryophylli
MSTDSLQQFRRRVAIPGPAGFGAAPLGNLFSRVTDEAAQETLSAAWGAGIRYFDTAPFYGMGLSERRLGRALASYPRDAFVISTKVGRLLIADDSVPETQHGYVGGLPYRVEYDYTADGARRSIESSLERLGLDRIDIVYIHDVAEDTHGPLWRQHYRTAAGGAMKALSRLRDEGVIGGWGLGVNRVEPCLMALADADPDVFLIAGRYTLLDTTALDALIPACEARGARLVVGGPYNSGLLAGGDTFEYARADAAMLARRARLLSHCERFGVDLKAAALQFCKAPGVVACVIAGARNADEVRQNCAAMTAPVPREFWQALKEEGLVPQGAPVPA

SEQ ID NO: 118 - DNA, 1119 bp, Artificial Sequence
  Codon-optimised sequence for Pl.LgdA
atgagtaatgccgaaaaagcactgggcgtggctttaatcggtaccggctttatgggcaaatgccatgccatggcatggcgcaatgtggccaccgcatttggtggtttaccgccgcgtttagaggtgctggcagatatgccggcagataaggcacattctttagcaagcagctttggctttgcccgcggtaccgcagattggcgtgaagccgttaatgatccggcagtggacgtggtgagcattaccaccccgaacggcttacatcgcgaaatggccgaagcagcactggcagctggtaaacatgtttggctggagaagccgatggctttaagtgtggaagatgcccaagctatggaagcagccgcccgtgcaagcgatcgtcgcaccatcatcggctacaactatactcgcagcccggcctttcgcgccgccgtggatctgattgcagaaggcgcaattggccgtccgattcactttcgcggcatgtatgacgaggactacatggccgatccggatctgccgtggagctggcgtctgacacgtaaagatggtggtttaggtgcactgggtgatctgggttgccatctggttagcgtgatggtgagcttaatgggtccggttgcacgcgtgtatgcacaagctgataccgtgattaccgatcgccctcatcaaggtggcacagcacgcgttgaaaatgaggatcaagctcaagctttaatccgcttcgcaagcggtaccagcggtgaatttagctgcagccgtgtggcccgcggttatcgttgccgtctggcatgggaagtgcaaggtaccgaaggtactttacgctttgatcaagaacgcatgaacgaactgtggctgtatcagccgggtcgcccggaaatcgatggctttcgccgcatcttaactggtccggcccagccgggctttgcagcattttgcccgggcggtggccataactttggctttaatgagcagaaagtggttgaagccgaaatgctgcgccaagctattgctggtcgcggtaaagcatggccggacttcaccgatggtctgaccattgaacgcgtgattcacggtatggcaaccagtgcccaaactggtcagccggtgaatttttaa

SEQ ID NO: 119 - protein, 372 aa, Paracoccus laeviglucosivorans
MSNAEKALGVALIGTGFMGKCHAMAWRNVATAFGGLPPRLEVLADMPADKAHSLASSFGFARGTADWREAVNDPAVDVVSITTPNGLHREMAEAALAAGKHVWLEKPMALSVEDAQAMEAAARASDRRTIIGYNYTRSPAFRAAVDLIAEGAIGRPIHFRGMYDEDYMADPDLPWSWRLTRKDGGLGALGDLGCHLVSVMVSLMGPVARVYAQADTVITDRPHQGGTARVENEDQAQALIRFASGTSGEFSCSRVARGYRCRLAWEVQGTEGTLRFDQERMNELWLYQPGRPEIDGFRRILTGPAQPGFAAFCPGGGHNFGFNEQKVVEAEMLRQAIAGRGKAWPDFTDGLTIERVIHGMATSAQTGQPVNF

SEQ ID NO: 120 - DNA, 990 bp, Artificial Sequence
  Codon-optimised sequence for Ps.Fdh
atgtcttctactgaacctgctgcagcagctgctggtctggccattccggcactgggttatggcgccgcaaacgtgggcaatttatttcgtgcactgagcgacgatgaagcatgggccgttctggaagcagcttgggatgccggcattcgttattatgacaccgccccgcattatggtttaggtctgagcgaaaagcgtctgggtgcatttttacagaccaagccgcgcgacgaatttgtggttagcaccaaagctggtcgtttattacgtcctaatccggaacgccgtccgagcggtctggataccgataacgactttcacgtgccggatgatctgcgccgcgaatgggatttcaccgaacaaggtattcgcgccagtattgccgagagtcaagaacgtctgggtctggatcgcattgatctgctgtatttacatgatccggaacgtcatgatctggatctggcactggcaagtgcctttccggctttagaaaaagtgcgcgccgaaggcgtggtgaaagccattggcattggcagcatggtgagtgatgcactgacccgcgccgttcgtgaagcagatttagatttaatcatggtggccggtcgctatactttattagaacagccggcagccaccgaagttctgccggcttgtgcagaaaatgccaccggcattgtggccgccagtgtgtttaatagcggtttactggcacagagtgagccgaaacgtgatggccgctatgagtatggccagctgccggatgaactgtgggatcgtctggtgcgcattgccgccatttgtcgcaaccatgatgttccgctgccggccgcagccatccagtttccgctgcagagtgctttagtgcgtagcgtggttgtgggtggtagtcgcccggcacagctgacccagaatgcagagtatgccgctttagaaattccggctggtttatgggcagaactggcagaagcccgcttaattccgaccccgtaa

SEQ ID NO: 121 - protein, 329 aa, Pseudomonas sp. 1143
MSSTEPAAAAAGLAIPALGYGAANVGNLFRALSDDEAWAVLEAAWDAGIRYYDTAPHYGLGLSEKRLGAFLQTKPRDEFVVSTKAGRLLRPNPERRPSGLDTDNDFHVPDDLRREWDFTEQGIRASIAESQERLGLDRIDLLYLHDPERHDLDLALASAFPALEKVRAEGVVKAIGIGSMVSDALTRAVREADLDLIMVAGRYTLLEQPAATEVLPACAENATGIVAASVFNSGLLAQSEPKRDGRYEYGQLPDELWDRLVRIAAICRNHDVPLPAAAIQFPLQSALVRSVVVGGSRPAQLTQNAEYAALEIPAGLWAELAEARLIPTP

SEQ ID NO: 122 - DNA, 1149 bp, Xanthomonas campestris
atgaatacacgtcgccaattcctgtctgctgccgcggccggcaccacggcgctggccgcatcgccattgctggcacagtcgtccgcacccggaagcgtgatgcccacgcgcggccgcacggcggcctctgccgtgcaaactagcgcgccggccaagggcgcacgttatcggccgaccacgcggttgggctttggcggtgttgcaatcggcaatgggtttgcgccagccaccgatgcgcaaagcgagcagacgctggcggcggtgtgggcctcgggcgtgcgctatttcgatacctcgccgtggtacgggctgggcctgtccgaacgccgcaccggccatcatctgcataaccatgccgccgatagctacgtgctctcgaccaaggtggggcgcttgctgaccgcgaccgacaagccgccgaagaccatgtggcagcaaccgtccccgttcgattaccgctacgactacagcgccgccggcgtgcgccgctcgatcgaagacagcctgcaacggctgggcgtgtcgcagatcgacatcgtgtacatccacgacctctcgccggagaacgaaaaagacctcggcatgccctgggaacagcgctttgccgaggccgccaagggcgcgatgccggagctgaccaagatgcgcaaggagggcctgatcaaggcctggggctttggcgtcaatcgcccggaaccggccttgcgtgcgattgaagaagccgacccggatatcttcttgctggcctgccagtactcgttgctcgaccacgcgcaggccttgcatgacaccttcccgaagatcgccagacacggcgcctcggtggtggtgggcgcgccgctgctggccggttatctggccgggcgcgaccgttatctgtacgagggcacggtgccggagtgggcgccgggcaaacgccagaaggccctggcaatctgcgacaaacacggcgtggatctgcgtacggtgtcgctgcaatttgctgccgcaccggaggtggtgtcggcggtgattcccggcgcacgcacggccgaacaggcgcaggccaatgcggcctcgatgcgggtggcgattcctgccgcgctctgggacgacctcaagcgcgcgcaggtgatcgaagcggatgcgccggtgccggcggctggatga

SEQ ID NO: 123 - protein, 382 aa, Xanthomonas campestris
MNTRRQFLSAAAAGTTALAASPLLAQSSAPGSVMPTRGRTAASAVQTSAPAKGARYRPTTRLGFGGVAIGNGFAPATDAQSEQTLAAVWASGVRYFDTSPWYGLGLSERRTGHHLHNHAADSYVLSTKVGRLLTATDKPPKTMWQQPSPFDYRYDYSAAGVRRSIEDSLQRLGVSQIDIVYIHDLSPENEKDLGMPWEQRFAEAAKGAMPELTKMRKEGLIKAWGFGVNRPEPALRAIEEADPDIFLLACQYSLLDHAQALHDTFPKIARHGASVVVGAPLLAGYLAGRDRYLYEGTVPEWAPGKRQKALAICDKHGVDLRTVSLQFAAAPEVVSAVIPGARTAEQAQANAASMRVAIPAALWDDLKRAQVIEADAPVPAAG

SEQ ID NO: 124 - DNA, 1014 bp, Acidovorax avenae
atgaaggtcacagaaacacgccgcatcggcgaaggccccgcacggctcacgcgcttcggcgtgggttgcggcacgctggccgggctgtacaagcccacgtccgaggcagacgcccgggccacgctgcaggcggcatgggatgccggcctgcgctacttcgacaccgcccctttctacgggcacacgctgagcgagcaccgcgtgggcgccttcctgcgcgagcagccccgcgatgccttcgtcatcagcaccaaggtcggccggctgatgcgccccgattcctccgtgcagccgatggacaacggctgggccgccccgctgccgttccgcccggactacgactacagctacggcggcgtcatgcgctcgttcgaggacagcctgcagcggctcggcctgcagcgcatcgacctgctgttcgtgcacgacatcggcacctacacccatggcgaccggaacgccgggcactggaaggccctcacggcacaaggcggcttccgcgcgctggaagagctccgggccgatggccgcatcgccggcttcggcctgggcgtcaacgaagcccaagtcatcctcgacagcctgcaggagacgcggctggactgctccctgctcgccgggcgctacacgctgctggagcagcatagcctgccgctcatgaacacatgccagcggctcggcagtgccatcgtcatcggcggtccgttcaactccgggctgctggtgggcaacggcaagttcgactatgcggatgcgccggcggacatgctggcgcgtgcccaggcgctggacgaggcgtgcgccgcgcacggcgtggccctgccagcggccgcgctgcaatttccgatggcccatggcgccgtggcatcgtgcgtctccggcgcgcgcaacccggcccagctggaccagaccgtgggctggttcgagcaggacattcccgcagcgctctgggccgacctgcagcagcaaggcctgatcgcggaaggctgccccgttcccggagccgcgccatga

SEQ ID NO: 125 - protein, 337 aa, Acidovorax avenae
MKVTETRRIGEGPARLTRFGVGCGTLAGLYKPTSEADARATLQAAWDAGLRYFDTAPFYGHTLSEHRVGAFLREQPRDAFVISTKVGRLMRPDSSVQPMDNGWAAPLPFRPDYDYSYGGVMRSFEDSLQRLGLQRIDLLFVHDIGTYTHGDRNAGHWKALTAQGGFRALEELRADGRIAGFGLGVNEAQVILDSLQETRLDCSLLAGRYTLLEQHSLPLMNTCQRLGSAIVIGGPFNSGLLVGNGKFDYADAPADMLARAQALDEACAAHGVALPAAALQFPMAHGAVASCVSGARNPAQLDQTVGWFEQDIPAALWADLQQQGLIAEGCPVPGAAP

SEQ ID NO: 126 - DNA, 1086 bp, Artificial Sequence
  Codon-optimised sequence for Ppi.TadH
atgaatcggcgcacaggcgcggcgagcgagacctatatcaacgttgcgaggcaatccgtgaacaacaggcaagccctgattccgccgggaccgttgggtttcggcgcggcccccttgggcaacctctttgcgcgcgtcagcgaagaagcggctgaagagacattgagcgcggcatgggaagccggcatgcgctatttcgataccgcgccgttctacggcatgggtctgtccgaacagcgcctcggccgcgcgctcgcgcaccgtgcgcgcgacgaattcgtcgtctcgacgaaagtgggccgcctgctgattcccgatgcgtccgttcccgaagtccagcacggctatgtcggcgcgctgccgtttcgcgtcgaattcgattattcggcgcaaggcgtgctccgctccatcgacgagagtctcaagcgtctttcactcgatcgcatcgatatcgtctatatccacgacgtcgccgaagatgcgcatcgtgaggactggctgcggcaataccgtatcgccgtcgaaggcgcgatgcgggcgctgagcgatctgcgcgatcagggcgtgatcggcgcatgggggctcggcgtcaaccgggtcgagccgtgcatgatgacgttgaacgacgcggaccctgacgtgtttctgatcgccggaagatacacgctgctcgacacgtcggcgctcgatgcgctgatcccggcttgcgatgctcgcggcgtgcagttggtcgcgggcggtccgttcaactcggggctgctcgtgggcggcacgacgttcgaatatgccgaggcgccggcggaaatgctcgagaagcggcgcagacttctcgcgcactgcgagcgcttcggcgtggacctgaaggcggccgcgctgcagttcacggatgcgccgcgagcggtcgcgtgcgtgatcgcgggcgcgagaaacgcgagcgagatcagacagaactgcgcggcaatgaccgagccgattccggcggcattctgggcggcattgaaggccgatggcctgattcccgccgatgcgccggagccccttgcttcaggcggcgcgctccacgcgacgacacacgagcccgtttag

SEQ ID NO: 127 - protein, 361 aa, Paraburkholderia piptadeniae
MNRRTGAASETYINVARQSVNNRQALIPPGPLGFGAAPLGNLFARVSEEAAEETLSAAWEAGMRYFDTAPFYGMGLSEQRLGRALAHRARDEFVVSTKVGRLLIPDASVPEVQHGYVGALPFRVEFDYSAQGVLRSIDESLKRLSLDRIDIVYIHDVAEDAHREDWLRQYRIAVEGAMRALSDLRDQGVIGAWGLGVNRVEPCMMTLNDADPDVFLIAGRYTLLDTSALDALIPACDARGVQLVAGGPFNSGLLVGGTTFEYAEAPAEMLEKRRRLLAHCERFGVDLKAAALQFTDAPRAVACVIAGARNASEIRQNCAAMTEPIPAAFWAALKADGLIPADAPEPLASGGALHATTHEPV

SEQ ID NO: 128 - DNA, 1050 bp, Artificial Sequence
  Codon-optimised sequence for Ss.Adh4
atggagaacgtgaatatggtgaaaagcaaggccgctttactgaagaaattcagcgaaccgctgagcatcgaggatgtgaacattcccgaaccgcaaggtgaagaagtgctgatccgcattggtggtgctggtgtgtgccgcaccgatctgcgtgtgtggaagggcgtggaagccaaacaaggtttccgtttaccgatcattctgggccatgaaaacgccggcacaatcgttgaagtgggcgagctggcaaaagtgaagaagggcgataacgtggtggtgtacgcaacttggggcgatttaacttgtcgctattgccgcgagggtaaatttaatatttgtaaaaaccaaattatcccgggccagaccaccaacggtggctttagcgagtacatgctggtgaaaagtagccgctggctggtgaaactgaacagtctgagccccgttgaagccgcacctctggccgatgccggtacaaccagcatgggcgccatccgtcaagcactgccgttcattagcaagttcgccgaaccggttgtgattgtgaatggtatcggcggtttagccgtgtacaccattcagattctgaaggctttaatgaaaaacattaccatcgtgggtatcagccgcagcaaaaagcatcgcgacttcgctttagaactgggcgccgattacgtgagcgaaatgaaggatgccgaatctttaatcaataagctgaccgacggtttaggtgcaagcatcgcaatcgatctggtgggtaccgaagagaccacctacaatttaggcaagctgctggcccaagaaggtgccattattttagtgggtatggaaggcaaacgcgtgagtctggaagccttcgacacagcagtgtggaacaagaaactgctgggcagcaactacggctctttaaatgatttagaagatgtggtgcgtctgagcgaaagcggcaaaattaagccgtacatcatcaaggtgccgctggatgacatcaacaaagccttcaccaatctggatgagggccgtgtggatggtcgccaagttatcaccccgtaa

SEQ ID NO: 129 - protein, 349 aa, Sulfolobus solfataricus
MENVNMVKSKAALLKKFSEPLSIEDVNIPEPQGEEVLIRIGGAGVCRTDLRVWKGVEAKQGFRLPIILGHENAGTIVEVGELAKVKKGDNVVVYATWGDLTCRYCREGKFNICKNQIIPGQTTNGGFSEYMLVKSSRWLVKLNSLSPVEAAPLADAGTTSMGAIRQALPFISKFAEPVVIVNGIGGLAVYTIQILKALMKNITIVGISRSKKHRDFALELGADYVSEMKDAESLINKLTDGLGASIAIDLVGTEETTYNLGKLLAQEGAIILVGMEGKRVSLEAFDTAVWNKKLLGSNYGSLNDLEDVVRLSESGKIKPYIIKVPLDDINKAFTNLDEGRVDGRQVITP

SEQ ID NO: 130 - DNA, 777 bp, Burkholderia multivorans
atggatctgaatctgcaggacaaggtcgtgatcgtgacgggcggcgcgtcgggcatcggcggcgcgatctcgatgcggctcgccgaagaacgcgcgattccggtcgtgttcgcgcgccacgcgcccgacggcgcgttcctcgacgcgctcgcgcaacggcagccgcgcgcgacgtatctgccggtcgagctgcaggacgatgcgcaatgccgcgacgcggtcgctcagacgatcgcgacgttcggacgcctcgacgggctcgtgaacaacgcgggcgtcaacgacggcatcgggctcgacgcagggcgcgacgcgttcgtcgcatcgctcgagcgcaacctgatccactactacgcgatggcgcattactgcgtgccgcatctgaaggcgacgcgcggcgcgatcgtcaacatctcgtcgaagacggccgtgaccgggcagggcaatacgagcggctattgcgcgtcgaagggcgcgcagctcgcgctgacgcgcgaatgggcggtcgcgctgcgcgagcacggcgtgcgcgtgaacgccgtgattccggccgaggtcatgacgccgctctaccggaactggatcgcgacgttcgaggatcccgaggcgaagctcgccgagatcgcggcgaaggtgccgctcggcaagcgcttcacgacacccgacgagatcgccgatacggccgtgttcctgctgtcgccgcgcgcgtcgcacacgacgggcgaatggctgttcgtcgacggcggctatacgcatctcgatcgtgcgctcgtctga

SEQ ID NO: 131 - protein, 258 aa, Burkholderia multivorans
MDLNLQDKVVIVTGGASGIGGAISMRLAEERAIPVVFARHAPDGAFLDALAQRQPRATYLPVELQDDAQCRDAVAQTIATFGRLDGLVNNAGVNDGIGLDAGRDAFVASLERNLIHYYAMAHYCVPHLKATRGAIVNISSKTAVTGQGNTSGYCASKGAQLALTREWAVALREHGVRVNAVIPAEVMTPLYRNWIATFEDPEAKLAEIAAKVPLGKRFTTPDEIADTAVFLLSPRASHTTGEWLFVDGGYTHLDRALV

SEQ ID NO: 132 - DNA, 1068 bp, Artificial Sequence
  Codon-optimised sequence for Tt.lac11
atgcgcaaactgcttggcagctctgccatccacgtgctgtcccgtcgcaccggttatggtatgtacttcctgattctggccctgggtgtcgctacacaggttgtctttggagcagaaccgagtcagaatccgagcgaaaaggtggcatggattgaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaa

SEQ ID NO: 133 - protein, 355 aa, Thermogutta terrifontis
MRKLLGSSAIHVLSRRTGYGMYFLILALGVATQVVFGAEPSQNPSEKVAWIERADPGLDDLIAPDAQVEVLAEGFEWSEGPVWIPEGGYLLFSDVPKNTIYRWKEGQGIDIFLKPSGYTGFRERGGESGSNGLALDRQGRLLLCQHGDRRVARWEKGCFITLADQYEGKPLNSPNDLVVKSNGDIYFTDPPYGMTPEAQRDPNALGFCGVYRISADGKLTLLVRDMTRPNGIAFSPDEKTLYVAQSDPQRPLWMAFPVREDGTLGEGKVFFDAKPWQQSGLPGLPDGMKVDQKGNLFATGPGGVNIFRPDGTFLGRIRVKVPTANCAFGDDGSTLYITADMYLLRVKTKTKGLGF

SEQ ID NO: 134 - DNA, 957 bp, Artificial Sequence
  Codon-optimised sequence for truncated variant of Tt.lac11
atggaaccgagtcagaatccgagcgaaaaggtggcatggattgaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaa

SEQ ID NO: 135 - protein, 318 aa, Artificial Sequence
  Variant of Tt.Lac11 truncated by 38 amino acids
MEPSQNPSEKVAWIERADPGLDDLIAPDAQVEVLAEGFEWSEGPVWIPEGGYLLFSDVPKNTIYRWKEGQGIDIFLKPSGYTGFRERGGESGSNGLALDRQGRLLLCQHGDRRVARWEKGCFITLADQYEGKPLNSPNDLVVKSNGDIYFTDPPYGMTPEAQRDPNALGFCGVYRISADGKLTLLVRDMTRPNGIAFSPDEKTLYVAQSDPQRPLWMAFPVREDGTLGEGKVFFDAKPWQQSGLPGLPDGMKVDQKGNLFATGPGGVNIFRPDGTFLGRIRVKVPTANCAFGDDGSTLYITADMYLLRVKTKTKGLGF

SEQ ID NO: 136 - DNA, 918 bp, Artificial Sequence
  Variant of Tt.Lac11 truncated by 38 amino acids
atggaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaa

SEQ ID NO: 137 - protein, 305 aa, Artificial Sequence
  Variant of Tt.Lac11 truncated by 51 amino acids
MERADPGLDDLIAPDAQVEVLAEGFEWSEGPVWIPEGGYLLFSDVPKNTIYRWKEGQGIDIFLKPSGYTGFRERGGESGSNGLALDRQGRLLLCQHGDRRVARWEKGCFITLADQYEGKPLNSPNDLVVKSNGDIYFTDPPYGMTPEAQRDPNALGFCGVYRISADGKLTLLVRDMTRPNGIAFSPDEKTLYVAQSDPQRPLWMAFPVREDGTLGEGKVFFDAKPWQQSGLPGLPDGMKVDQKGNLFATGPGGVNIFRPDGTFLGRIRVKVPTANCAFGDDGSTLYITADMYLLRVKTKTKGLGF

SEQ ID NO: 138 - DNA, 843 bp, Artificial Sequence
  Codon-optimised sequence for truncated variant of Tt.lac11
atgtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaa

SEQ ID NO: 139 - protein, 280 aa, Artificial Sequence
  Variant of Tt.Lac11 truncated by 76 amino acids
MWSEGPVWIPEGGYLLFSDVPKNTIYRWKEGQGIDIFLKPSGYTGFRERGGESGSNGLALDRQGRLLLCQHGDRRVARWEKGCFITLADQYEGKPLNSPNDLVVKSNGDIYFTDPPYGMTPEAQRDPNALGFCGVYRISADGKLTLLVRDMTRPNGIAFSPDEKTLYVAQSDPQRPLWMAFPVREDGTLGEGKVFFDAKPWQQSGLPGLPDGMKVDQKGNLFATGPGGVNIFRPDGTFLGRIRVKVPTANCAFGDDGSTLYITADMYLLRVKTKTKGLGF

SEQ ID NO: 140 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 141 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 142 - DNA, 1677 bp, Artificial Sequence
  Codon-optimised sequence for Ss.IlvD
atgccggcaaaattaaattcaccgagccgttatcatggtatttacaatgcaccgcaccgcgccttcttacgtagcgtgggtttaacagatgaggagatcggcaaaccgctggtggcaatcgccaccgcatggagcgaagctggtccgtgtaatttccacactttagctttagcccgtgttgccaaagagggtaccaaggaagctggtctgagccctctggcattcccgaccatggtggtgaacgacaatatcggcatgggcagcgaaggcatgcgttattctctggtgagtcgcgatctgatcgccgatatggtggaggcccagtttaacgcccacgcattcgatggcttagtgggcatcggcggctgcgataagaccaccccgggcattttaatggccatggcccgtttaaatgtgccgagcatttatatttacggcggtagcgccgaaccgggctattttatgggcaagcgtttaacaatcgaggacgttcatgaagccatcggcgcatatttagccaagcgcattacagaaaacgaactgtatgaaattgaaaaacgcgcccaccctacactgggtacttgtagcggtctgttcaccgccaacaccatgggtagtatgagcgaagcactgggcatggctttaccgggtagtgcaagtccgacagcaacaagcagccgccgtgttatgtatgtgaaagagaccggcaaagcactgggttctttaattgaaaatggcatcaagagccgcgagattttaacctttgaagcattcgagaacgccatcaccactttaatggctatgggtggtagcacaaatgcagtgctgcatctgctggccattgcatacgaagccggcgtgaaactgactttagatgacttcaaccgcatcagcaagcgtacaccgtatatcgccagcatgaaacccggtggtgattatgtgatggccgatttagatgaagttggcggcgtgccggtggtgctgaaaaaactgctggacgccggtttactgcatggcgatgttctgaccgtgaccggtaagaccatgaagcagaatttagagcagtacaagtaccctaacgtgccgcatagccatatcgtgcgtgatgtgaagaacccgatcaaaccgcgcggcggtatcgttattttaaaaggctctttagcccccgaaggcgcagttattaaagtggccgccaccaacgtggttaagttcgaaggcaaggccaaggtgtacaatagcgaggacgatgccttcaaaggcgtgcaaagtggcgaggtgagtgagggtgaagttgtgatcatccgctatgagggtccgaaaggcgcaccgggtatgcccgaaatgctgcgtgttaccgccgccatcatgggtgccggtttaaacaatgtggcactggttaccgacggtcgttttagcggcgcaacacgcggtccgatggttggtcatgttgcaccggaggcaatggttggcggcccgatcgcaattgtggaagacggtgacaccattgtgatcgatgtggagagtgagcgtttagatctgaaactgagcgaagaggagatcaaaaatcgtctgaaacgttggagcccgccgagccctcgctacaaaagtggtttactggcaaaatacgccagtctggttagtcaagctagcatgggcgcagtgacccgtcccgcttaa

SEQ ID NO: 143 - protein, 558 aa, Sulfolobus solfataricus
MPAKLNSPSRYHGIYNAPHRAFLRSVGLTDEEIGKPLVAIATAWSEAGPCNFHTLALARVAKEGTKEAGLSPLAFPTMVVNDNIGMGSEGMRYSLVSRDLIADMVEAQFNAHAFDGLVGIGGCDKTTPGILMAMARLNVPSIYIYGGSAEPGYFMGKRLTIEDVHEAIGAYLAKRITENELYEIEKRAHPTLGTCSGLFTANTMGSMSEALGMALPGSASPTATSSRRVMYVKETGKALGSLIENGIKSREILTFEAFENAITTLMAMGGSTNAVLHLLAIAYEAGVKLTLDDFNRISKRTPYIASMKPGGDYVMADLDEVGGVPVVLKKLLDAGLLHGDVLTVTGKTMKQNLEQYKYPNVPHSHIVRDVKNPIKPRGGIVILKGSLAPEGAVIKVAATNVVKFEGKAKVYNSEDDAFKGVQSGEVSEGEVVIIRYEGPKGAPGMPEMLRVTAAIMGAGLNNVALVTDGRFSGATRGPMVGHVAPEAMVGGPIAIVEDGDTIVIDVESERLDLKLSEEEIKNRLKRWSPPSPRYKSGLLAKYASLVSQASMGAVTRPA

SEQ ID NO: 144 - DNA, 1326 bp, Artificial Sequence
  Codon-optimised sequence for Xc.FucD
atgcgtaccattatcgctttagaaacccatgatgttcgctttccgacaagccgtgagctggatggcagcgacgcaatgaacccggacccggactatagcgccgcctacgttgttctgcgtacagatggcgcagaagatctggccggctacggtctggtgtttaccattggtcgcggcaacgatgttcagacagcagccgtggcagctttagcagaacacgtggtgggtttaagcgttgacaaggtgattgccgatctgggtgcatttgcacgccgtttaaccaatgatagccaactgcgttggctgggtccggaaaaaggcgtgatgcacatggccatcggcgccgttattaatgccgcatgggatctggcagcacgcgccgcaaataaaccgctgtggcgctttatcgccgaactgaccccggaacagctggtggacaccatcgactttcgctatctgagcgatgcactgacccgtgatgaagcactggcaattctgcgtgatgcacagccgcagcgtgcagcacgtaccgccacactgatcgagcaaggttatccggcatataccaccagcccgggctggctgggctacagtgacgaaaaattagtgcgtttagccaaggaagccgttgccgatggcttccgcaccattaaactgaaagtgggcgccaacgtgcaagatgatattcgccgttgccgtctggcacgtgccgcaattggtccggacatcgccatggcagtggatgccaaccagcgctgggatgttggtccggccattgattggatgcgccagctggccgagtttgatatcgcttggattgaggaaccgacaagtccggatgatgtgctgggccatgcagcaatccgtcaaggtatcaccccggttccggtgagtaccggcgaacacacccagaaccgcgttgttttcaaacagctgctgcaagctggtgccgtggatctgattcaaatcgatgccgcccgtgttggcggcgttaatgagaatttagccattttactgctggccgccaaattcggcgtgcgtgtgtttccgcatgctggtggcgtgggtctgtgcgaactggttcagcatctggcaatggccgatttcgttgcaattaccggcaagatggaggatcgtgccatcgagtttgtggatcatctgcaccagcactttttagacccggtgcgcattcagcacggtcgctatctggccccggaagttccgggtttcagcgccgaaatgcacccggcaagcattgccgagtttagctacccggacggccgtttctgggttgaagatttagccgccagcaaagccaaagcctaa

SEQ ID NO: 145 - protein, 441 aa, Xanthomonas campestris
MRTIIALETHDVRFPTSRELDGSDAMNPDPDYSAAYVVLRTDGAEDLAGYGLVFTIGRGNDVQTAAVAALAEHVVGLSVDKVIADLGAFARRLTNDSQLRWLGPEKGVMHMAIGAVINAAWDLAARAANKPLWRFIAELTPEQLVDTIDFRYLSDALTRDEALAILRDAQPQRAARTATLIEQGYPAYTTSPGWLGYSDEKLVRLAKEAVADGFRTIKLKVGANVQDDIRRCRLARAAIGPDIAMAVDANQRWDVGPAIDWMRQLAEFDIAWIEEPTSPDDVLGHAAIRQGITPVPVSTGEHTQNRVVFKQLLQAGAVDLIQIDAARVGGVNENLAILLLAAKFGVRVFPHAGGVGLCELVQHLAMADFVAITGKMEDRAIEFVDHLHQHFLDPVRIQHGRYLAPEVPGFSAEMHPASIAEFSYPDGRFWVEDLAASKAKA

SEQ ID NO: 146 - DNA, 1236 bp, Pseudomonas putida
atgaacagtgcccccgactattcggcggcctacgtggtgctgcacaccgacgctgccgccctcgaaggccacggcctgaccttcaccatcggccgtggcaacgaaatatgcgcagcagcggtgcagtcgctggcccccttgatcgtcggccttacgctcgaagaaatcagcgccgacatgggcgctttctggcatcgctttaccgtcagcgacagccagttgcgctggctgggcccggaaaagggcgtgatccacctggccaccgccgccatcatcaacgcagtgtgggacctgtgggccaagcacgaaggcaagcccgtgtggaaactgctggccgacatgacgccggagcagctggtgcgctgcctggacttcagctacgtcaccgacgtgctcacccccgaagaggccatcgccctgctgcgccgccaggcgccgggcaaagccgagcgcgaggcacacatgctgcgtgaaggctaccccgggtacaccactgccccgggctggctgggctacagcgaagagaaaatgcgcaaactggcccgcgaggccgtggcagacggctggacccacatcaaacagaagatcggggccgacctggaagaagacatccgccgcgccagcatcctgcgcgacgaaatcggctgggaacgcaccctgatgatggatgccaaccaagtgtggggcgtcgaagagtcggtggccaacatgcgccgcctggccgcattcgaaccgctttggatcgaagaaccgaccagccccgatgacattcttggccacgccaccattcgccagcgcattgcgccaatcggtgtcgccaccggtgagcattgccacaaccgggtgatgttcaagcagatgttccaggccggtgcactggacttctgccagctggacgcagcccgcctgggtggcctgaacgaggtcctgatcgtgctgctgatggccgccaagtacgacgtgccggtctgcccgcacggcggtggcgtcgggctgtgcgaatatgtgcagaacatcgcgcttttcgactacatcgcggtttctgcctcgctgcacaaccgggtgctggagtacgtcgaccacctgcacgagcacttcatcgacccggtggtgatccatcgcgggcgctacatgccgccacaacgccctggctacagcatcgagatgcacgctgagaccctggaacgttaccagtaccccaacggcgccgtgtggctcgacatcaaccgttcctga

SEQ ID NO: 147 - protein, 411 aa, Pseudomonas putida
MNSAPDYSAAYVVLHTDAAALEGHGLTFTIGRGNEICAAAVQSLAPLIVGLTLEEISADMGAFWHRFTVSDSQLRWLGPEKGVIHLATAAIINAVWDLWAKHEGKPVWKLLADMTPEQLVRCLDFSYVTDVLTPEEAIALLRRQAPGKAEREAHMLREGYPGYTTAPGWLGYSEEKMRKLAREAVADGWTHIKQKIGADLEEDIRRASILRDEIGWERTLMMDANQVWGVEESVANMRRLAAFEPLWIEEPTSPDDILGHATIRQRIAPIGVATGEHCHNRVMFKQMFQAGALDFCQLDAARLGGLNEVLIVLLMAAKYDVPVCPHGGGVGLCEYVQNIALFDYIAVSASLHNRVLEYVDHLHEHFIDPVVIHRGRYMPPQRPGYSIEMHAETLERYQYPNGAVWLDINRS

SEQ ID NO: 148 - DNA, 1170 bp, Bradyrhizobium japonicum
atgtccgtccgcatcgtcgacgtccgcgagatcaccaagccgatctcatcgccgatccgcaacgcctatatcgatttcaccaagatgacgacgagcctcgtcgccgtcgtcactgacgttgtgcgcgaaggcaagcgcgtcgtcggctacggcttcaattccaacggccgctacgggcagggcggcctgatccgcgaacgcttcgcctcgcgcatcctggaggccgatccgaagaagcttctgaacgaggccggcgacaatctcgatcccgacaaggtctgggccgcgatgatgatcaacgagaagccgggcggccatggcgagcgctcggtcgcggtcggcaccatcgacatggcggtgtgggacgcggtggcgaagatcgcgggcaagccgctgtttcgcctgctcgccgaacgtcacggcgtcaaagccaacccgcgcgtgttcgtctatgccgccggcggctactactatcccggcaaggggctatcgatgctgcgcggcgagatgcgtggctatctcgaccgcggctacaacgtcgtgaagatgaagatcggcggcgcgccgatcgaggaggatcgcatgcgcatcgaggcggtgctggaggagatcggcaaggacgcgcagctcgccgtcgacgccaacggccgcttcaacctcgagaccggcatcgcctatgccaagatgctgcgcgattatccgctgttctggtacgaggaggtcggcgatcccctcgactacgcgctgcaggccgcgctcgccgaattctatcccggcccgatggcgacaggcgaaaacctgttcagccaccaggacgcgcgcaacctgctccgctatggcggcatgcgcccggaccgcgactggctgcaattcgactgcgcgctgtcctatggcctctgcgaataccagcgcacgctcgaagtgctgaagacccatggctggtcgccaagccgctgcatcccgcatggcggccaccagatgtcgctcaacatcgccgctggcctcggcctcggcggcaacgaaagctacccggacctgttccagccctacggcggcttccccgacggcgtgcgcgtcgagaacggccacatcaccatgccggaccttccgggcatcggcttcgaaggcaagtcggatctctacaaggagatgaaggcgctggcggagtaa

SEQ ID NO: 149 - protein, 389 aa, Bradyrhizobium japonicum
MSVRIVDVREITKPISSPIRNAYIDFTKMTTSLVAVVTDVVREGKRVVGYGFNSNGRYGQGGLIRERFASRILEADPKKLLNEAGDNLDPDKVWAAMMINEKPGGHGERSVAVGTIDMAVWDAVAKIAGKPLFRLLAERHGVKANPRVFVYAAGGYYYPGKGLSMLRGEMRGYLDRGYNVVKMKIGGAPIEEDRMRIEAVLEEIGKDAQLAVDANGRFNLETGIAYAKMLRDYPLFWYEEVGDPLDYALQAALAEFYPGPMATGENLFSHQDARNLLRYGGMRPDRDWLQFDCALSYGLCEYQRTLEVLKTHGWSPSRCIPHGGHQMSLNIAAGLGLGGNESYPDLFQPYGGFPDGVRVENGHITMPDLPGIGFEGKSDLYKEMKALAE

SEQ ID NO: 150 - DNA, 1539 bp, Acidovorax avenae
atgtcgactgatgcactggcgacgcagtcgctgctcatcctgaacgaccgcgacaacgtggctgtcgcgcgcaccgaactcggcgcgggcatggagcttcggctggccggcggcgaagtactggaggtgatggccactgtgcccgccggccacaagatcgcactcaggcccatcgcgaagggcgatgccgtgctgaagtacggccaggtcatcggcgctgccagcacgccgatcggggccggcgagcacgtgcatgtgcacaacgtcgagatgggcgactccagccccacccacctggtgggccgcgcctacgtcccgaccgatcccgtggagcagcctgcgaccttcatgggctacgtcagggccgatgggcgcgtcggcacgcgcaactacatcggcgtcatttcttcggtgaactgctccgccacggtctgcaaggccatcgcgcaggccttcacgggcccggcgctcggtgcctatccgggtgtggacggtgtcgtcgccatcacgcacggcaccggctgcggcatgagcggtgaaggcgaaggcatcgaactcctgcgccgcacgctcagcggctacatcgcccaccccaatttcgccgcggtgctggtgatcggactgggctgcgaggtcaaccaggtcgagggcctgaccggccgcctgccgttgccggaaggccgggtgcgcaccatgaccatccaggacgccggtggcacccgcgaggccatcgcccagggcatcgatatcgtgcgcgacatggcagcggcggcatccggcgcccggcgcactcaggtgccggcatcgcacctcgtggtggggctgcagtgcggcggatcggacggctactcgggcatcagcgccaacccggtactgggcgcggcggtcgacatgctggtgcgccacggcggcacggccatcctgtcggaaacacccgagatctatggcgccgagcatttgctgacggcgcgcgccgcatcggccgaagtggcagaacgcctgatggcgcgcctggcctggtgggaggcctacacccagcgcaacggcgcgaacatgaacaacaacccctcgcccggcaacaaggccggcggcatcaccaccatcctcgagaaatcgctgggcgcggtggccaagggcggtggcagcgggctgatggatgtggtcgaatacgcgcagcccgtgcacagccggggcctggtgttcatggacacccccggctacgatccggtgtcggccaccggccaggtcgcgggcggtgccaacctcatctgtttcactaccgggcggggatcgacctacgggtgcaagcccacgccgtcgctcaagctcgccaccaacaccgcgctgttccgcaacatgggcatggacatggacttcaacgccggcgacgtggtggacggaacgctgggcatcgcgcaggcgggccgccatcttttcgaactgatgctcgccacggcgtccggcgaccgcacgctgagcgagacgcacggtctgggcgacaacgaattcgtgccctggcagctcggcgcggtgatgtga

SEQ ID NO: 151 - protein, 512 aa, Acidovorax avenae
MSTDALATQSLLILNDRDNVAVARTELGAGMELRLAGGEVLEVMATVPAGHKIALRPIAKGDAVLKYGQVIGAASTPIGAGEHVHVHNVEMGDSSPTHLVGRAYVPTDPVEQPATFMGYVRADGRVGTRNYIGVISSVNCSATVCKAIAQAFTGPALGAYPGVDGVVAITHGTGCGMSGEGEGIELLRRTLSGYIAHPNFAAVLVIGLGCEVNQVEGLTGRLPLPEGRVRTMTIQDAGGTREAIAQGIDIVRDMAAAASGARRTQVPASHLVVGLQCGGSDGYSGISANPVLGAAVDMLVRHGGTAILSETPEIYGAEHLLTARAASAEVAERLMARLAWWEAYTQRNGANMNNNPSPGNKAGGITTILEKSLGAVAKGGGSGLMDVVEYAQPVHSRGLVFMDTPGYDPVSATGQVAGGANLICFTTGRGSTYGCKPTPSLKLATNTALFRNMGMDMDFNAGDVVDGTLGIAQAGRHLFELMLATASGDRTLSETHGLGDNEFVPWQLGAVM

SEQ ID NO: 152 - DNA, 1536 bp, Herbaspirillum huttiense
atgaaagccaactctcccgtcatccgcctgaacccggtcgatgatgtcgtcatcgcccgccagcagctcatctccggcaccgtgctgcaggacgaaggcggcctcaaagtccagggcctcattcccgccggccacaagatggcgacccgcgccatctctgcgggcgagccggtcaagcgctatggccagatcatcggcacggccagccaggacatcgcgcccggacagcacgtgcacacgcacaacctggccatggccgagttctcgcgtgaacaccatttcggcgctgacgtgaagccggtcgatttcgtcgccgagcccgcaaccttcatgggcatcgtgcgtcccgatggccgcgtggccacgcgcaactacatcggcgtactgacctcggtgaactgctcggccaccgcggcacgcgccatcgccgattacttccgccgcgacatccatcccgaagtgctggccgactatcccaacatcgatggcgtggtggccctgacccatggccagggttgcgccacggattcgcagggcgagccgctgcagatcctgcgccgcacgctggcgggttacgccacgcatcccaacttcgctgcggtgctggtggtgggcctgggctgcgagaccaaccagatctcgggcctgatggaaagccacaacctgaaggaaggcgaatacttccacaccttcaccatccagggcaccggcggcaccgccaagaccgtggcgctggggatcgagaagatcaagaagatgctgcccaaggccaatgacatcaagcgcgagccggtgtcggccaagcatctgacgctgggcctgcagtgcggtggttcggatggctattcgggcatcacggccaatccggcgctgggtgcggcagtggacttgctggtgcggcatggcggcacggccatcctttccgaaacgccggaaatctatggggccgaacacctgctcacccgccgcgcggtctcgccggaagtcggtgagaagctgctggcccgcatcgcctggtgggaggagtattgcgccaagaacgatgccgagatgaacaacaatccctcagcgggcaacaaggccggtggcttgaccaccatcctcgagaagtcgctgggcgcggttgccaagggtggcactaccaatctggtggatgtctacaagtatgcggagacggtcaccgcacgcggcttcgtcttcatggatacgccgggctacgacccgatctccgccaccggccaggtcgccggaggggccaacatgatctgcttcaccacgggccggggttctgcctatggctgcgcaccggcaccgtcgctcaagctggccaccaatactgcgctgtggcaacgtcaggaagaagacatggatatcaactgcggcgagattgccgacagcaatgtcaccccgcaggaggtcggcgaacgcttcttccagatgatcctcgatacggcatcgggcaagaagaccaagagcgagctgcatggctacgggcaagacgaattcgtgccctggcacatcggcgtctacacctga

SEQ ID NO: 153 - protein, 511 aa, Herbaspirillum huttiense
MKANSPVIRLNPVDDVVIARQQLISGTVLQDEGGLKVQGLIPAGHKMATRAISAGEPVKRYGQIIGTASQDIAPGQHVHTHNLAMAEFSREHHFGADVKPVDFVAEPATFMGIVRPDGRVATRNYIGVLTSVNCSATAARAIADYFRRDIHPEVLADYPNIDGVVALTHGQGCATDSQGEPLQILRRTLAGYATHPNFAAVLVVGLGCETNQISGLMESHNLKEGEYFHTFTIQGTGGTAKTVALGIEKIKKMLPKANDIKREPVSAKHLTLGLQCGGSDGYSGITANPALGAAVDLLVRHGGTAILSETPEIYGAEHLLTRRAVSPEVGEKLLARIAWWEEYCAKNDAEMNNNPSAGNKAGGLTTILEKSLGAVAKGGTTNLVDVYKYAETVTARGFVFMDTPGYDPISATGQVAGGANMICFTTGRGSAYGCAPAPSLKLATNTALWQRQEEDMDINCGEIADSNVTPQEVGERFFQMILDTASGKKTKSELHGYGQDEFVPWHIGVYT

SEQ ID NO: 154 - DNA, 1536 bp, Artificial Sequence
  Sequence for mutant of Hh.araD
atgaaagccaactctcccgtcatccgcctgaacccggtcgatgatgtcgtcatcgcccgccagcagctcatctccggcaccgtgctgcaggacgaaggcggcctcaaagtccagggcctcattcccgccggccacaagatggcgacccgcgccatctctgcgggcgagccggtcaagcgctatggccagatcatcggcacggccagccaggacatcgcgcccggacagcacgtgcacacgcacaacctggccatggccgagttctcgcgtgaacaccatttcggcgctgacgtgaagccggtcgatttcgtcgccgagcccgcaaccttcatgggcatcgtgcgtcccgatggccgcgtggccacgcgcaactacatcggcgtactgacctcggtgaactgctcggccaccgcggcacgcgccatcgccgattacttccgccgcgacatccatcccgaagtgctggccgactatcccaacatcgatggcgtggtggccctgacccatggccagggttgcgccacggattcgcagggcgagccgctgcagatcctgcgccgcacgctggcgggttacgccacgcatcccaacttcgctgcggtgctggtggtgggcctgggctgcgagaccaaccagatctcgggcctgatggaaagccacaacctgaaggaaggcgaatacttccacaccttcaccatccagggcaccggcggcaccgccaagaccgtggcgctggggatcgagaagatcaagaagatgctgcccaaggccaatgacatcaagcgcgagccggtgtcggccaagcatctgacgctgggcctgcagtgcggtggttcggatggctattcgggcatcacggccaatccggcgctgggtgcggcagtggacttgctggtgcggcatggcggcacggccatcctttccgaaacgccggaaatctatggggccgaacacctgctcacccgccgcgcggtctcgccggaagtcggtgagaagctgctggcccgcatcgcctggtgggaggagtattgcgccaagaacgatgccgagatgaacaacaatccctcagcgggcaacaaggccggtggcttgaccaccatcctcgagaagtcgctgggcgcggttgccaagggtggcactaccaatctggtggatgtctacaagtatgcggagacggtcaccgcacgcggcttcgtcttcatggatacgccgggctacgacccgatctccgccaccggccaggtcgccggaggggccaacatgatctgcttcaccacgggccggggttctgcctatggcagcgcaccggcaccgtcgctcaagctggccaccaatactgcgctgtggcaacgtcaggaagaagacatggatatcaactgcggcgagattgccgacagcaatgtcaccccgcaggaggtcggcgaacgcttcttccagatgatcctcgatacggcatcgggcaagaagaccaagagcgagctgcatggctacgggcaagacgaattcgtgccctggcacatcggcgtctacacctga

SEQ ID NO: 155 - protein, 511 aa, Artificial Sequence
  Mutant of Hh.araD with mutation of cysteine to serine at position 434
MKANSPVIRLNPVDDVVIARQQLISGTVLQDEGGLKVQGLIPAGHKMATRAISAGEPVKRYGQIIGTASQDIAPGQHVHTHNLAMAEFSREHHFGADVKPVDFVAEPATFMGIVRPDGRVATRNYIGVLTSVNCSATAARAIADYFRRDIHPEVLADYPNIDGVVALTHGQGCATDSQGEPLQILRRTLAGYATHPNFAAVLVVGLGCETNQISGLMESHNLKEGEYFHTFTIQGTGGTAKTVALGIEKIKKMLPKANDIKREPVSAKHLTLGLQCGGSDGYSGITANPALGAAVDLLVRHGGTAILSETPEIYGAEHLLTRRAVSPEVGEKLLARIAWWEEYCAKNDAEMNNNPSAGNKAGGLTTILEKSLGAVAKGGTTNLVDVYKYAETVTARGFVFMDTPGYDPISATGQVAGGANMICFTTGRGSAYGSAPAPSLKLATNTALWQRQEEDMDINCGEIADSNVTPQEVGERFFQMILDTASGKKTKSELHGYGQDEFVPWHIGVYT

SEQ ID NO: 156 - DNA, 1479 bp, Clostridium acetobutylicum
atgaaaaatgttataaagataaatgaaaaagataatgttgtagttgctttaaatgatttaaacaaaggcgatgtaattgaaatagatggaaaagtaattacagcagaagagcctgtaaaaaaaggtcataaaattgcaattacagatatacaaaagaactctaatatatataagtatggatttcctattggtcatgcgttagaagaaataaaaaaaggtcaatgggtacatactcataatataaaaaccaatttggatggaataaaggattatgagtacaataaacaaacttttgagaatccatttaaaaatgaaaatttaacttttaagggttatagaagagaagacggaacagtaggaataagaaatgaattatggatagttccaactgttggttgtgtgaatggtacagctgatttaatagctgaaagatttaaaagtgaaactgagtttaaagatgtacatgtatttaagcataattttggatgttcacagcttggtgatgatcacaataacacaagaactatattaggtaacattgtaaagcatcctaatgcaggaggagtattagttcttggacttggttgcgaaaataatactatggagagtttcaaggaatcacttcatagctacaataaagaaagagtaagatttttaatagctcaagatgtagaagatgaaatatcttccggatgtgagttgttaaaagaattatatgaaaagatacaaaaggatgaaagagaagaggtatctatttcagaacttaaaataggacttaaatgtggagcatcagatggtttttctggaataactgcaaatccacttttaggaaagttatcagactttttaatagcacagggtggaacaactatacttacagaagttcctgaaatgtttggagcagaaacaatactcatgaatagggcaaaagatgagaaagtatttgcaaaaacggtaaacttgattaatgattttaaaaaatatttcatgagctataatcaacctgtatatgaaaatccttctccaggaaacaaagcaggtggaataactacattagaagataaatcattaggatgtacacaaaaatcaggaagctctgaagtagttggagtacttaagtacggagaaactttagaaaataaagggttaaatcttttaagtgcgccaggtaatgatttagtggcatctactgctcttgcatcggcaggatgtcatatggtgttgtttacaactggtagaggtacaccatttggaacttttgtgccaactgttaagatttcaactaactcagatatatataataagaagaaaaattggatagattttaatgcgggtgcacttttagaaaatcaatctatggatcaagtgcttaaagaatttattaattatttactcggagttgcaaacggaaatatggcaaacaatgagaaaaataacataagagaaatatctatatttaaaaacggtgtaacactataa

SEQ ID NO: 157 - protein, 492 aa, Clostridium acetobutylicum
MKNVIKINEKDNVVVALNDLNKGDVIEIDGKVITAEEPVKKGHKIAITDIQKNSNIYKYGFPIGHALEEIKKGQWVHTHNIKTNLDGIKDYEYNKQTFENPFKNENLTFKGYRREDGTVGIRNELWIVPTVGCVNGTADLIAERFKSETEFKDVHVFKHNFGCSQLGDDHNNTRTILGNIVKHPNAGGVLVLGLGCENNTMESFKESLHSYNKERVRFLIAQDVEDEISSGCELLKELYEKIQKDEREEVSISELKIGLKCGASDGFSGITANPLLGKLSDFLIAQGGTTILTEVPEMFGAETILMNRAKDEKVFAKTVNLINDFKKYFMSYNQPVYENPSPGNKAGGITTLEDKSLGCTQKSGSSEVVGVLKYGETLENKGLNLLSAPGNDLVASTALASAGCHMVLFTTGRGTPFGTFVPTVKISTNSDIYNKKKNWIDFNAGALLENQSMDQVLKEFINYLLGVANGNMANNEKNNIREISIFKNGVTL

SEQ ID NO: 158 - DNA, 1530 bp, Paraburkholderia mimosarum
atgaagacctcaacagcagacgcagatgtcaccgtgcgactgcatcccgatgacgatgtgatcatcgcgctgcagcagcttgtgggtggcacaacggtggacggtggccgcgtaaccgtccgggggctggtggcccctggtcacaaggtcgcctcacgtgccatccagcaaggcgagccggttcgacggtacgggcagatcatcggctttgcctcgtgcgacatcggctgcggcgaccacgtccacacgcataacctgtcgatgggcagcttcgaccgcgactatgcgtttggaaaggacgcaaagccagcgcccgtcgcgagccgtgcgagcttcatgggtatcgtccgtcctgatggacgcgtcgcgacgcgcaattacatcggcattcttacgtcagtgaactgctctgcgaccgtcgcacgtgccattgcagaccacttcaaacctgaagtgctacgcgactatccaaacgtggacggggtcgtcgcactcacgcatggccagggttgcgcgatcgactcacaaggagaaggcctggcactgatccgtcgaacgctcggtgggtacgcacgacatcctaatttcgcggcggtgctcatcgtcggactcgggtgcgagaccaaccagattgggggcctgctcgaaagtcagggtcttgaggcgggcagcacgctgaaggtcatgaccattcaggctaccggtggcacgtcgcgcacagtggcggctggcatcgacgctgtgcaggcgctgctgccagaggccaatcgcgtgaagcgggaggcagtcgacgcaagtaatctcgtggttggtttgcagtgcggcgggtccgacggctattcgggtatctcggcgaatccggcgcttggtgctgcggtcgaccgtcttgttgcccacggcggcgttgccattctgtccgagacaccggaaatatacggcgcggagcacctgctcacccgccgcgccatttcacgcgcggtcggagaaaagctgatcgagcgcattcactggtgggaaacttattgtgcacgcatgggcgcagacctgaataacaacccgtcggcgggtaataaggcaggtggcctgacgacaattctcgagaagtcgttgggcgctgtggccaaaggcggcacgacgaacctgattgctgtttatcgctatgcggagccggtatcggcacacggtctggttttcatggacaccccgggctatgaccccgtctcggcaaccggccaggtagcaggcggagccaacctgatctgctttacaacggggcgcggctctgcatacggctgcgcgccttcgccctcgctgaaactgggcaccaacagcgaactatggcgcagacaggaagaagatatcgacctgaactgcggagagatcctcgacggaacgaggagcgtcgacgaaatgggccaggagattttcgagctcatgctggcaaccgcatcgggtatgcgatcaaagagtgagcttcacggctacggacaaaatgagttcgtaccctggcagatcggcgcgatcacctga

SEQ ID NO: 159 - protein, 509 aa, Paraburkholderia mimosarum
MKTSTADADVTVRLHPDDDVIIALQQLVGGTTVDGGRVTVRGLVAPGHKVASRAIQQGEPVRRYGQIIGFASCDIGCGDHVHTHNLSMGSFDRDYAFGKDAKPAPVASRASFMGIVRPDGRVATRNYIGILTSVNCSATVARAIADHFKPEVLRDYPNVDGVVALTHGQGCAIDSQGEGLALIRRTLGGYARHPNFAAVLIVGLGCETNQIGGLLESQGLEAGSTLKVMTIQATGGTSRTVAAGIDAVQALLPEANRVKREAVDASNLVVGLQCGGSDGYSGISANPALGAAVDRLVAHGGVAILSETPEIYGAEHLLTRRAISRAVGEKLIERIHWWETYCARMGADLNNNPSAGNKAGGLTTILEKSLGAVAKGGTTNLIAVYRYAEPVSAHGLVFMDTPGYDPVSATGQVAGGANLICFTTGRGSAYGCAPSPSLKLGTNSELWRRQEEDIDLNCGEILDGTRSVDEMGQEIFELMLATASGMRSKSELHGYGQNEFVPWQIGAIT

SEQ ID NO: 160 - DNA, 1488 bp, Escherichia coli
atgcaatacatcaagatccatgcgctggataacgtcgcggtcgctttagcagatttggctgaaggcacagaagtcagtgtcgataaccagactgttacgctgcgccaggatgttgctcgtggacataaatttgcgttaacggatatcgcaaaaggggccaatgtcatcaaatatggcctgccgattggttatgcattggcggatattgcggcaggtgttcacgttcacgcccacaatacgcgcacgaatctgagcgatctggatcagtatcgctatcaacctgattttcaggatctgcctgcgcaagcggcagatcgtgaagtgcagatctatcgtcgcgctaacggcgatgtcggggtgcgtaatgagctgtggatcctgccaaccgtgggctgtgtcaacggcatcgcgcggcagatccagaaccgttttctgaaagagaccaacaacgccgaaggtactgacggcgtgttcctcttcagccacacctacggctgctcacagctgggcgacgatcacattaatacccgcaccatgctgcaaaacatggtgcgccacccgaacgcaggcgcagtgctggtgattggtctgggctgtgaaaacaaccaggttgccgcattccgtgaaacgctgggcgatatcgatcctgaacgcgttcatttcatgatctgccaacagcaggatgatgagatcgaagccggaatcgagcatttgcatcagctgtataacgtgatgcgcaacgataaacgcgagccaggcaaactcagcgaactgaagtttggtctggagtgcggtggttctgacggtctttctggtattactgctaacccgatgctggggcgtttctctgactacgtgattgctaacggcggtactaccgtactgaccgaagtgccggagatgtttggcgcagagcagttgctgatggaccattgccgcgacgaagcaacgtttgaaaaactggtcaccatggtcaacgacttcaaacagtactttattgcccatgatcagccgatctatgaaaacccatcgccggggaacaaagcgggcggtatcaccacgctggaagacaaatcacttggctgtacccagaaagcgggttccagcgtcgtggttgacgtgctgcgttacggcgagcgtctgaaaacgccagggctgaacttgttaagtgcgccgggtaacgatgccgtagcgaccagcgccctggcgggtgcgggctgccatatggtgctgttcagtactggtcgtggcacgccgtatggtggatttgtgccgacggtgaaaatcgccaccaacagtgaactggcggcgaagaaaaaacactggatcgactttgacgcgggtcagttgatccacggtaaagcgatgccgcagttgctggaagaatttatcgacaccatcgttgagtttgccaacggtaagcaaacctgtaacgagcgtaacgacttccgcgaactggcgatcttcaaaagcggcgtaacgctataa

SEQ ID NO: 161 - protein, 495 aa, Escherichia coli
MQYIKIHALDNVAVALADLAEGTEVSVDNQTVTLRQDVARGHKFALTDIAKGANVIKYGLPIGYALADIAAGVHVHAHNTRTNLSDLDQYRYQPDFQDLPAQAADREVQIYRRANGDVGVRNELWILPTVGCVNGIARQIQNRFLKETNNAEGTDGVFLFSHTYGCSQLGDDHINTRTMLQNMVRHPNAGAVLVIGLGCENNQVAAFRETLGDIDPERVHFMICQQQDDEIEAGIEHLHQLYNVMRNDKREPGKLSELKFGLECGGSDGLSGITANPMLGRFSDYVIANGGTTVLTEVPEMFGAEQLLMDHCRDEATFEKLVTMVNDFKQYFIAHDQPIYENPSPGNKAGGITTLEDKSLGCTQKAGSSVVVDVLRYGERLKTPGLNLLSAPGNDAVATSALAGAGCHMVLFSTGRGTPYGGFVPTVKIATNSELAAKKKHWIDFDAGQLIHGKAMPQLLEEFIDTIVEFANGKQTCNERNDFRELAIFKSGVTL

SEQ ID NO: 162 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 163 - protein, 312 aa, Artificial Sequence
  Mutant of Ec.Mdh with mutations I12V R81A M85Q D86S G179D
MKVAVLGAAGGVGQALALLLKTQLPSGSELSLYDIAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 164 - DNA, 984 bp, Cupriavidus necator
atgcagatttctatcaaacgcgccatcgagcgcgtgcccggcggcatgatgatcgtgccgctgctgatcggttcgctcgttgccacgttcgcgccggatgggccgaagttttttggatcgtttaccggcgcgctttttaccggtgccctgcccatcctcgcggtgttctatgtctgcatgggggcaagcatcaacatcaaagccacgccttacattctcaagaagggcggcgtgttgttcggcaccaaggtcggcacggcaatggtgttgggcgtgatcatggggcacttcctgggcgaggcgccgatcagtgccgggatgtttgccggcctgtcgacgcttgccgtggtggccgcgatgaacgacaccaacggcggcctgtacatggcgctgatgggccagtacggcaagcctgaagacgtgggcgcctacagcatcatgtcgctcgagtccggcccgttcctgacgatggtgacgctgggcgtggcgggtttgtccgcgttcccatggccgacgatggtcggcagcattcttccgctcatgctcggcatgttgctgggcaacctggatcgggagatgcgcgatttcctgggtaaggccgtgccggtcatgattccctttttcgcactggcgctgggcgcaggcctggacctgcacaaggtctggcaggcgggcatgctgggcctcggccttggtgttgccgtggtggccgtcaccggctgcgcgctgtacttcgctgaccgcctcaccggcggcacgggtgtggcaggtgtcgcagccgccagcaccgccggcaatgccgccgccgtgccgacgctggtagcggcggctaatccggtctacgcagaatcggccaagagcgccacgatcctggtggccgcctgcgtggtggtcaccgccgtactgacgccattggtgaccgcctggattgcccgccgcgtggcgggcaaggaggccgaggcgttggtgaggaccgccgcatga

SEQ ID NO: 165 - protein, 327 aa, Cupriavidus necator
MQISIKRAIERVPGGMMIVPLLIGSLVATFAPDGPKFFGSFTGALFTGALPILAVFYVCMGASINIKATPYILKKGGVLFGTKVGTAMVLGVIMGHFLGEAPISAGMFAGLSTLAVVAAMNDTNGGLYMALMGQYGKPEDVGAYSIMSLESGPFLTMVTLGVAGLSAFPWPTMVGSILPLMLGMLLGNLDREMRDFLGKAVPVMIPFFALALGAGLDLHKVWQAGMLGLGLGVAVVAVTGCALYFADRLTGGTGVAGVAAASTAGNAAAVPTLVAAANPVYAESAKSATILVAACVVVTAVLTPLVTAWIARRVAGKEAEALVRTAA

SEQ ID NO: 166 - DNA, 1191 bp, Escherichia coli
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 167 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 168 - DNA, 7978 bp, Artificial Sequence
  Sequence for plasmid
ggatccgtttaactttaagaaggagatataccatggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgtatggaaccgcggtatatggcgcagcacaagggctgctgtcggcgctggcaggtgcggaatatgttggcccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaaatgtttaactttaagaaggagatataccatgtctaccgatagtttacaacagtttcgtcgccgtgtggcaattccgggtccggctggttttggtgcagcaccgctgggcaatctgtttagccgtgttaccgatgaggccgcacaagaaactttaagtgccgcatggggtgctggtatccgctactttgataccgcaccgttctatggcatgggtctgagtgaacgccgtctgggtcgcgcactggccagctatccgcgtgatgccttcgtgattagcaccaaagtgggtcgtttactgatcgccgacgacagtgtgccggaaacccagcacggttatgttggtggtttaccgtaccgcgttgaatacgactatactgccgatggcgcccgccgtagcattgaaagctctttagaacgtttaggtttagatcgtattgacattgtgtatattcacgacgtggccgaggatacccatggtccgctgtggcgtcagcattatcgtaccgccgctggtggtgcaatgaaagctttaagccgtttacgcgatgaaggtgtgattggtggctggggtttaggcgtgaatcgtgttgaaccgtgtctgatggctttagccgatgccgatccggacgtctttttaatcgccggccgctatactttactggataccacagctttagatgctttaattccggcatgcgaagcacgcggtgcacgtttagttgttggtggtccgtacaatagtggtttactggctggtggcgatacctttgaatatgcccgcgcagatgccgcaatgttagcccgtcgcgcacgtttattaagccactgcgaacgctttggtgttgatttaaaggccgccgctttacagttttgtaaagccccgggcgttgttgcatgcgttattgccggtgcccgcaatgcagatgaggttcgtcagaactgcgcagcaatgacagcaccggtgccgcgcgaattttggcaagctttaaaggaagagggcttagttccgcaaggtgcaccggttccggcataatctagagtttaactttaagaaggagatataccatggaaccgagtcagaatccgagcgaaaaggtggcatggattgaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaagtcgacctgcaggcatgcaagcttctgttttggcggatgagagaagaaattcgtcgcccgccataaactgccaggcatcaaattaagcagaaggccatcctgacggatggcctttttgcgtttctacaaactcttcctgtctagcaggtggcacttttcggggaaatgtgcgcggaacccctatttgtttatttttctaaatacattcaaatatgtatccgctcatgctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcgagctcggtacccgg

SEQ ID NO: 169 - DNA, 8122 bp, Artificial Sequence
  Sequence for plasmid
ggatccgtttaactttaagaaggagatataccatggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgtatggaaccgcggtatatggcgcagcacaagggctgctgtcggcgctggcaggtgcggaatatgttggcccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaaatgtttaactttaagaaggagatataccatgaatacacgtcgccaattcctgtctgctgccgcggccggcaccacggcgctggccgcatcgccattgctggcacagtcgtccgcacccggaagcgtgatgcccacgcgcggccgcacggcggcctctgccgtgcaaactagcgcgccggccaagggcgcacgttatcggccgaccacgcggttgggctttggcggtgttgcaatcggcaatgggtttgcgccagccaccgatgcgcaaagcgagcagacgctggcggcggtgtgggcctcgggcgtgcgctatttcgatacctcgccgtggtacgggctgggcctgtccgaacgccgcaccggccatcatctgcataaccatgccgccgatagctacgtgctctcgaccaaggtggggcgcttgctgaccgcgaccgacaagccgccgaagaccatgtggcagcaaccgtccccgttcgattaccgctacgactacagcgccgccggcgtgcgccgctcgatcgaagacagcctgcaacggctgggcgtgtcgcagatcgacatcgtgtacatccacgacctctcgccggagaacgaaaaagacctcggcatgccctgggaacagcgctttgccgaggccgccaagggcgcgatgccggagctgaccaagatgcgcaaggagggcctgatcaaggcctggggctttggcgtcaatcgcccggaaccggccttgcgtgcgattgaagaagccgacccggatatcttcttgctggcctgccagtactcgttgctcgaccacgcgcaggccttgcatgacaccttcccgaagatcgccagacacggcgcctcggtggtggtgggcgcgccgctgctggccggttatctggccgggcgcgaccgttatctgtacgagggcacggtgccggagtgggcgccgggcaaacgccagaaggccctggcaatctgcgacaaacacggcgtggatctgcgtacggtgtcgctgcaatttgctgccgcaccggaggtggtgtcggcggtgattcccggcgcacgcacggccgaacaggcgcaggccaatgcggcctcgatgcgggtggcgattcctgccgcgctctgggacgacctcaagcgcgcgcaggtgatcgaagcggatgcgccggtgccggcggctggatgatctagagtttaactttaagaaggagatataccatggaaccgagtcagaatccgagcgaaaaggtggcatggattgaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaagtcgacctgcaggcatgcaagcttctgttttggcggatgagagaagaaattcgtcgcccgccataaactgccaggcatcaaattaagcagaaggccatcctgacggatggcctttttgcgtttctacaaactcttcctgtctagcaggtggcacttttcggggaaatgtgcgcggaacccctatttgtttatttttctaaatacattcaaatatgtatccgctcatgctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcgagctcggtacccgg

SEQ ID NO: 170 - DNA, 9033 bp, Artificial Sequence
  Sequence for plasmid
ggtaccgtttaactttaagaaggagatataccatggctgatacaatgctcgccgccgtcgtccgtgaattcggcaagccgctctccatcgagcggctacccatcccggacatcaagccccaccagatcctcgtgaaggtcgatacctgtggcgtctgccacactgacctgcacgccgcgcgcggggactggccgtccaagcccaacccgccgttcattcccgggcatgaaggcgtcggacacatcgtcgccgtcggcagtcaggtcggcgatttcgtcaagaccggcgatgtcgtgggcgtgccctggctctactccgcctgcggtcactgcgaacactgtctgggcggctgggaaacactctgcgaaaagcaggacgacaccggctacaccgtcaatggctgcttcgccgaatatgtcgtggcagacccgaactacgtcgcacacctgccctcgaccatcgacccgcttcaggcctcgccggtcctgtgcgcggggctgacggtctataagggcctgaaaatgacggaggcccgccccggccagtgggtcgcagtctcgggcgtcggcggtctcggccagatggccgtgcagtacgccgtcgccatgggcatgaatgtcgtcgcggtggacatcgatgacgaaaaactcgccacagccaaaaagctcggcgcatccctgaccgtcaacgccaaggacacggacccggccaggttcatccagcagcagatcggcggcgcacatggcgctctcgtcaccgctgtcggacggacggcgttttcgcaggccatgggctatgcccgccgcggcggcaccatcgtcctgaacggactgccgcccggcgatttcccggtctcgatcttcgacatggtcatgaacggcaccaccatccgtggctccatcgtcggaacacggctggacatgatcgaggccatggatttcttcgcccgcggcaaggtcaaatccgtcgtcacccccggaaaacttgaaaacatcaatacgatcttcgacgatctgcagaatggtcgcctcgaaggccggacagtgctcgacttccggtcctaaggatccgtttaactttaagaaggagatataccatggaactgtatctggatacttcagacgttgttgcggtgaaggcgctgtcacgtatttttccgctggcgggtgtgaccactaacccaagcattatcgccgcgggtaaaaaaccgctggatgttgtgcttccgcaacttcatgaagcgatgggcggtcaggggcgtctgtttgcccaggtaatggctaccactgccgaagggatggttaatgacgcgcttaagctgcgttctattattgcggatatcgtggtgaaagttccggtgaccgccgaggggctggcagctattaagatgttaaaagcggaagggattccgacgtatggaaccgcggtatatggcgcagcacaagggctgctgtcggcgctggcaggtgcggaatatgttggcccttacgttaatcgtattgatgctcagggcggtagcggcattcagactgtgaccgacttacaccagttattgaaaatgcatgcgccgcaggcgaaagtgctggcagcgagtttcaaaaccccgcgtcaggcgctggactgcttactggcaggatgtgaatcaattactctgccactggatgtggcacaacagatgattagctatccggcggttgatgccgctgtggcgaagtttgagcaggactggcagggagcgtttggcagaacgtcgatttaaatgtttaactttaagaaggagatataccatgtctaccgatagtttacaacagtttcgtcgccgtgtggcaattccgggtccggctggttttggtgcagcaccgctgggcaatctgtttagccgtgttaccgatgaggccgcacaagaaactttaagtgccgcatggggtgctggtatccgctactttgataccgcaccgttctatggcatgggtctgagtgaacgccgtctgggtcgcgcactggccagctatccgcgtgatgccttcgtgattagcaccaaagtgggtcgtttactgatcgccgacgacagtgtgccggaaacccagcacggttatgttggtggtttaccgtaccgcgttgaatacgactatactgccgatggcgcccgccgtagcattgaaagctctttagaacgtttaggtttagatcgtattgacattgtgtatattcacgacgtggccgaggatacccatggtccgctgtggcgtcagcattatcgtaccgccgctggtggtgcaatgaaagctttaagccgtttacgcgatgaaggtgtgattggtggctggggtttaggcgtgaatcgtgttgaaccgtgtctgatggctttagccgatgccgatccggacgtctttttaatcgccggccgctatactttactggataccacagctttagatgctttaattccggcatgcgaagcacgcggtgcacgtttagttgttggtggtccgtacaatagtggtttactggctggtggcgatacctttgaatatgcccgcgcagatgccgcaatgttagcccgtcgcgcacgtttattaagccactgcgaacgctttggtgttgatttaaaggccgccgctttacagttttgtaaagccccgggcgttgttgcatgcgttattgccggtgcccgcaatgcagatgaggttcgtcagaactgcgcagcaatgacagcaccggtgccgcgcgaattttggcaagctttaaaggaagagggcttagttccgcaaggtgcaccggttccggcataatctagagtttaactttaagaaggagatataccatggaaccgagtcagaatccgagcgaaaaggtggcatggattgaacgcgcagatcccggcctcgacgatctgattgctccagatgcgcaagtggaagtgctggcagaaggttttgagtggagcgaaggtccggtatggattcccgaaggcggctacttgctgtttagcgatgttccgaagaacaccatttaccgctggaaagaaggtcaggggattgacatcttcctgaaaccgtcgggatataccggctttcgtgaacgtggtggtgaatcgggctcaaacggcttagccctggatcgccaaggccgccttctgttgtgtcagcatggtgatcgtcgtgtagctcgttgggagaaaggttgctttatcacactggcagaccagtatgagggcaaaccgctgaattccccgaatgacttggtagtcaagtcgaacggcgatatctacttcacggatccaccatatggcatgactcctgaggcccaacgcgatcccaatgcgctcggcttttgcggcgtttatcgcattagtgcggatgggaagctgacgctgttagttcgtgacatgactcgccctaacgggatcgccttcagcccagacgagaaaacgctctatgttgcccagtcagatccgcaacgcccgttatggatggcgttcccggttcgcgaagatgggaccttaggggaaggcaaagtgttcttcgatgcgaaaccgtggcagcaaagtggcttgccgggactcccagatggcatgaaagtggaccagaaaggcaatctgtttgcgactggtccgggtggtgtcaacatctttcgccctgatggtacctttctgggccggattcgggtgaaagtccctaccgctaactgtgcgtttggtgatgacggatctacgctttacattaccgcggacatgtatctgcttcgtgtgaaaacgaaaaccaaaggactggggttctaagtcgacctgcaggcatgcaagcttctgttttggcggatgagagaagaaattcgtcgcccgccataaactgccaggcatcaaattaagcagaaggccatcctgacggatggcctttttgcgtttctacaaactcttcctgtctagcaggtggcacttttcggggaaatgtgcgcggaacccctatttgtttatttttctaaatacattcaaatatgtatccgctcatgctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcgagctc

SEQ ID NO: 171 - DNA, 8592 bp, Artificial Sequence
  Sequence for plasmid
gagctcgtttaactttaagaaggagatataccatgaaagtcgcagtcctcggcgccgctggcggtgtcggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaaggatccgtttaactttaagaaggagatataccatgaaagccaactctcccgtcatccgcctgaacccggtcgatgatgtcgtcatcgcccgccagcagctcatctccggcaccgtgctgcaggacgaaggcggcctcaaagtccagggcctcattcccgccggccacaagatggcgacccgcgccatctctgcgggcgagccggtcaagcgctatggccagatcatcggcacggccagccaggacatcgcgcccggacagcacgtgcacacgcacaacctggccatggccgagttctcgcgtgaacaccatttcggcgctgacgtgaagccggtcgatttcgtcgccgagcccgcaaccttcatgggcatcgtgcgtcccgatggccgcgtggccacgcgcaactacatcggcgtactgacctcggtgaactgctcggccaccgcggcacgcgccatcgccgattacttccgccgcgacatccatcccgaagtgctggccgactatcccaacatcgatggcgtggtggccctgacccatggccagggttgcgccacggattcgcagggcgagccgctgcagatcctgcgccgcacgctggcgggttacgccacgcatcccaacttcgctgcggtgctggtggtgggcctgggctgcgagaccaaccagatctcgggcctgatggaaagccacaacctgaaggaaggcgaatacttccacaccttcaccatccagggcaccggcggcaccgccaagaccgtggcgctggggatcgagaagatcaagaagatgctgcccaaggccaatgacatcaagcgcgagccggtgtcggccaagcatctgacgctgggcctgcagtgcggtggttcggatggctattcgggcatcacggccaatccggcgctgggtgcggcagtggacttgctggtgcggcatggcggcacggccatcctttccgaaacgccggaaatctatggggccgaacacctgctcacccgccgcgcggtctcgccggaagtcggtgagaagctgctggcccgcatcgcctggtgggaggagtattgcgccaagaacgatgccgagatgaacaacaatccctcagcgggcaacaaggccggtggcttgaccaccatcctcgagaagtcgctgggcgcggttgccaagggtggcactaccaatctggtggatgtctacaagtatgcggagacggtcaccgcacgcggcttcgtcttcatggatacgccgggctacgacccgatctccgccaccggccaggtcgccggaggggccaacatgatctgcttcaccacgggccggggttctgcctatggctgcgcaccggcaccgtcgctcaagctggccaccaatactgcgctgtggcaacgtcaggaagaagacatggatatcaactgcggcgagattgccgacagcaatgtcaccccgcaggaggtcggcgaacgcttcttccagatgatcctcgatacggcatcgggcaagaagaccaagagcgagctgcatggctacgggcaagacgaattcgtgccctggcacatcggcgtctacacctgatctagagtcgacctgcaggcatgcaagcttctgttttggcggatgagagaagaaattcgtcgcccgccataaactgccaggcatcaaattaagcagaaggccatcctgacggatggcctttttgcgtttctacaaactcttcctgtctagcaggtggcacttttcggggaaatgtgcgcggaacccctatttgtttatttttctaaatacattcaaatatgtatccgctcatgagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatattgattacgtgtttgaatcattgcatcgcctgtaaatttttaacttgtcctatttttgtcattaccacgtatatacacatgtataacaattcagatatcgttaccaggatatgccgcatcagcggcatggaaggcggcactctgttgtttcatatgatacaggagtaaaaccgccgaagcccggcgtaagccggtactgattgatagatttcaccttacccatccccagccctgccagaccatacccgctttcagccatgagagagcttctgtgcgcggtcggagtggtcccgacgagggtttacccgaagtcggggcgtatctccgcgttagcgggccgtcagggccgcttacgagcgtgtactgagaacttccagcgagaagactgacagcgatgatgaagtagttacaacattcataattaaaagcgactctgttccggcccttcgggccggggcggggccgcttttcagttatgagggaggggctttgtggtttcagttctgcgctggaccggggtttttctggtggttgtgtctgagtgttgtaactaaagcggctccggtaggggcccgccgcttgcggtgggaggtgcatatctgtctgtccacaggacaggcagtgaataggttttctttttaaatgaatgtaattaagtagtttaaaggagatataaacaggtgtttaaaagatacattgcaccctgtaacgctggcggatggcgctttatgacatgaacggttgtaaccttatggggaagtcccttgcagtttaatgtggataagcaaaattccccgtctgtgaggcgtgttttgtatcgaaaacagggggggccggatgcaccagaagctggatgatgaggttgttttttgtatgtggtgctgattttttgtgcgctgtcgggcttcagacgtgcggaagcctccggcgcaggccggattattcagaggaggtcactttcagggggaagctgtggccagccggctgtaattacggttacgtgacagaatcatgcgctccttcacacgacgctcaacttcgcgttttaccgcctcacgattggcagtgaagcgcccttccgcgatttcgcgcgtcagctgccgtttcaccagggtgacaatatcctgacgttccctgtccgcatcacgacgcgcacgggcacgctttattccccgggacttaagctctgtctgataactgcgaaaacgctcacgaacaaaacgccaggctttcgctatcagttcatccatgcccagggtatccagcccctgctttttgcgttgtttgttttcccataccacacggctgcggcgcgcggcggccactgcctcctctgatacatcgagggcagcaaacagtgcagatgtgaacgtgatatcggtcggaatgtagcacccgataagcgggtcatattccgtctggtaggtaatcagtcccagctctgacaggaacgtcagggcccgggtggcacgggtgatggagagttttccggcagcagactccgtcgccagtccgcactcaatggccagcgtggtgatggagcactggacgcggttggccagcgggtcatagtggaaacacagcccctgcaggagcgcatcaatagcccgtcgacgcagcactggtggcatgcgtcgacgcagaccacgcgaacgggcatgcgccacatgaatggcgaaatcaaaacgggaagtgaagcccaccgccttttccatcagtttttcgcagaacttcagcgttcctgcaccttcacggggtgtaaacaccggattcgggttctttacctggcggtaatacgtttggtgaagatcagtcacaccatcctgcacttacaatgcgcagaaggagcgagcacagaaagaagtcttgaacttttccgggcatataactatactccccgcatagctgaattgttggctatacggtttaagtgggccccggtaatcttttcgtactcgcaaaagttgaagaagattatcggggtttttgcttttctggctcctgtaaatccacatcagaaccagttccttgccaccttacggcgtggccagccacaaaattccttaaacgatcagtaatctagcactaatcttctgaacactcaagaatgtaagcccatcatcacatgcatcgttttgcgcttcactttttatcagtgcggtcagaacttcagcctgagtcagaccatcttcatgacacatttgcatgagcatggccttatactttggttcaagaaatacttttacttccttgaacgaagctcttttacgggccactgataatctttgtttctctgcatcagaaagcggattcccctttctgtatgctcgttttgcgccagatgaggaagtcactgcattttctgtctgcgacatctcgcctcctcaatactcaaacagggatcgtttcgcagaggatactacagttttttgaaatcagcgacttgagaattgtgacgaagatctgtgttggcgcacaaatcaacggggattactgtcgtttaatgtgatttaaactgtgaaatagtatggttttcagttattgaaacgccgggagcggggaaaacttgctttttcccgtttccggggttggacaactgagcaacgcgaaggcgtcagctacgatgttccggggactgctgatcagcccctgttgtattcagccattctccggtcatccgaacgccagcaagacgtagcccagcgcgtcggccaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattc

SEQ ID NO: 172 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatggcatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 173 - protein, 312 aa, Artificial Sequence
  Mutant of Ec.Mdh with mutations I12V R81A M85Q D86S G179D D34G
MKVAVLGAAGGVGQALALLLKTQLPSGSELSLYGIAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 174 - DNA, 939 bp, Artificial Sequence
  Sequence for mutant of Ec.Mdh
atgaaagtcgcagtcctcggcgctgctggcggtgtcggccaggcgcttgcactactgttaaaaacccaactgccttcaggttcagaactctctctgtatgattctgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggtcagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 175 - protein, 312 aa, Artificial Sequence
  Mutant of Ec.Mdh with mutations I12V R81A M85Q D86S G179D I35S
MKVAVLGAAGGVGQALALLLKTQLPSGSELSLYDSAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 176 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatggcaaagctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 177 - protein, 312 aa, Artificial Sequence
  Mutant of Ec.Mdh with mutations I12V R81A M85Q D86S G179D D34G I35K
MKVAVLGAAGGVGQALALLLKTQLPSGSELSLYGKAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 178 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatggccgtgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 179 - protein, 312 aa, Artificial Sequence
  Mutant of Ec.Mdh with mutations I12V R81A M85Q D86S G179D D34G I35R
MKVAVLGAAGGVGQALALLLKTQLPSGSELSLYGRAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 180 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatggctctgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 181 - protein, 312 aa, Artificial Sequence
  Mutant of Ec.Mdh with mutations I12V R81A M85Q D86S G179D D34G I35S
MKVAVLGAAGGVGQALALLLKTQLPSGSELSLYGSAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 182 - DNA, 939 bp, Artificial Sequence
  Sequence for mutant of Ec.Mdh
atgaaagtcgcagtcctcggcgccgctggcggtgtcggccaggcgcttgcactactgttaaaaacccaactgccttcaggttcagaactctctctgtatggcaccgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 183 - protein, 312 aa, Artificial Sequence
  Mutant of Ec.Mdh with mutations I12V R81A M85Q D86S G179D D34G I35T
MKVAVLGAAGGVGQALALLLKTQLPSGSELSLYGTAPVTPGVAVDLSHIPTAVKIKGFSGEDATPALEGADVVLISAGVAAKPGQSRSDLFNVNAGIVKNLVQQVAKTCPKACIGIITNPVNTTVAIAAEVLKKAGVYDKNKLFGVTTLDIIRSNTFVAELKGKQPGEVEVPVIGGHSDVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 184 - DNA, 8592 bp, Artificial Sequence
  Sequence for plasmid
gagctcgtttaactttaagaaggagatataccatgaaagtcgcagtcctcggcgccgctggcggtgtcggccaggcgcttgcactactgttaaaaacccaactgccttcaggttcagaactctctctgtatggccgtgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcggctaaaccgggccagtctcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggccactctgatgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaaggatccgtttaactttaagaaggagatataccatgaaagccaactctcccgtcatccgcctgaacccggtcgatgatgtcgtcatcgcccgccagcagctcatctccggcaccgtgctgcaggacgaaggcggcctcaaagtccagggcctcattcccgccggccacaagatggcgacccgcgccatctctgcgggcgagccggtcaagcgctatggccagatcatcggcacggccagccaggacatcgcgcccggacagcacgtgcacacgcacaacctggccatggccgagttctcgcgtgaacaccatttcggcgctgacgtgaagccggtcgatttcgtcgccgagcccgcaaccttcatgggcatcgtgcgtcccgatggccgcgtggccacgcgcaactacatcggcgtactgacctcggtgaactgctcggccaccgcggcacgcgccatcgccgattacttccgccgcgacatccatcccgaagtgctggccgactatcccaacatcgatggcgtggtggccctgacccatggccagggttgcgccacggattcgcagggcgagccgctgcagatcctgcgccgcacgctggcgggttacgccacgcatcccaacttcgctgcggtgctggtggtgggcctgggctgcgagaccaaccagatctcgggcctgatggaaagccacaacctgaaggaaggcgaatacttccacaccttcaccatccagggcaccggcggcaccgccaagaccgtggcgctggggatcgagaagatcaagaagatgctgcccaaggccaatgacatcaagcgcgagccggtgtcggccaagcatctgacgctgggcctgcagtgcggtggttcggatggctattcgggcatcacggccaatccggcgctgggtgcggcagtggacttgctggtgcggcatggcggcacggccatcctttccgaaacgccggaaatctatggggccgaacacctgctcacccgccgcgcggtctcgccggaagtcggtgagaagctgctggcccgcatcgcctggtgggaggagtattgcgccaagaacgatgccgagatgaacaacaatccctcagcgggcaacaaggccggtggcttgaccaccatcctcgagaagtcgctgggcgcggttgccaagggtggcactaccaatctggtggatgtctacaagtatgcggagacggtcaccgcacgcggcttcgtcttcatggatacgccgggctacgacccgatctccgccaccggccaggtcgccggaggggccaacatgatctgcttcaccacgggccggggttctgcctatggctgcgcaccggcaccgtcgctcaagctggccaccaatactgcgctgtggcaacgtcaggaagaagacatggatatcaactgcggcgagattgccgacagcaatgtcaccccgcaggaggtcggcgaacgcttcttccagatgatcctcgatacggcatcgggcaagaagaccaagagcgagctgcatggctacgggcaagacgaattcgtgccctggcacatcggcgtctacacctgatctagagtcgacctgcaggcatgcaagcttctgttttggcggatgagagaagaaattcgtcgcccgccataaactgccaggcatcaaattaagcagaaggccatcctgacggatggcctttttgcgtttctacaaactcttcctgtctagcaggtggcacttttcggggaaatgtgcgcggaacccctatttgtttatttttctaaatacattcaaatatgtatccgctcatgagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatattgattacgtgtttgaatcattgcatcgcctgtaaatttttaacttgtcctatttttgtcattaccacgtatatacacatgtataacaattcagatatcgttaccaggatatgccgcatcagcggcatggaaggcggcactctgttgtttcatatgatacaggagtaaaaccgccgaagcccggcgtaagccggtactgattgatagatttcaccttacccatccccagccctgccagaccatacccgctttcagccatgagagagcttctgtgcgcggtcggagtggtcccgacgagggtttacccgaagtcggggcgtatctccgcgttagcgggccgtcagggccgcttacgagcgtgtactgagaacttccagcgagaagactgacagcgatgatgaagtagttacaacattcataattaaaagcgactctgttccggcccttcgggccggggcggggccgcttttcagttatgagggaggggctttgtggtttcagttctgcgctggaccggggtttttctggtggttgtgtctgagtgttgtaactaaagcggctccggtaggggcccgccgcttgcggtgggaggtgcatatctgtctgtccacaggacaggcagtgaataggttttctttttaaatgaatgtaattaagtagtttaaaggagatataaacaggtgtttaaaagatacattgcaccctgtaacgctggcggatggcgctttatgacatgaacggttgtaaccttatggggaagtcccttgcagtttaatgtggataagcaaaattccccgtctgtgaggcgtgttttgtatcgaaaacagggggggccggatgcaccagaagctggatgatgaggttgttttttgtatgtggtgctgattttttgtgcgctgtcgggcttcagacgtgcggaagcctccggcgcaggccggattattcagaggaggtcactttcagggggaagctgtggccagccggctgtaattacggttacgtgacagaatcatgcgctccttcacacgacgctcaacttcgcgttttaccgcctcacgattggcagtgaagcgcccttccgcgatttcgcgcgtcagctgccgtttcaccagggtgacaatatcctgacgttccctgtccgcatcacgacgcgcacgggcacgctttattccccgggacttaagctctgtctgataactgcgaaaacgctcacgaacaaaacgccaggctttcgctatcagttcatccatgcccagggtatccagcccctgctttttgcgttgtttgttttcccataccacacggctgcggcgcgcggcggccactgcctcctctgatacatcgagggcagcaaacagtgcagatgtgaacgtgatatcggtcggaatgtagcacccgataagcgggtcatattccgtctggtaggtaatcagtcccagctctgacaggaacgtcagggcccgggtggcacgggtgatggagagttttccggcagcagactccgtcgccagtccgcactcaatggccagcgtggtgatggagcactggacgcggttggccagcgggtcatagtggaaacacagcccctgcaggagcgcatcaatagcccgtcgacgcagcactggtggcatgcgtcgacgcagaccacgcgaacgggcatgcgccacatgaatggcgaaatcaaaacgggaagtgaagcccaccgccttttccatcagtttttcgcagaacttcagcgttcctgcaccttcacggggtgtaaacaccggattcgggttctttacctggcggtaatacgtttggtgaagatcagtcacaccatcctgcacttacaatgcgcagaaggagcgagcacagaaagaagtcttgaacttttccgggcatataactatactccccgcatagctgaattgttggctatacggtttaagtgggccccggtaatcttttcgtactcgcaaaagttgaagaagattatcggggtttttgcttttctggctcctgtaaatccacatcagaaccagttccttgccaccttacggcgtggccagccacaaaattccttaaacgatcagtaatctagcactaatcttctgaacactcaagaatgtaagcccatcatcacatgcatcgttttgcgcttcactttttatcagtgcggtcagaacttcagcctgagtcagaccatcttcatgacacatttgcatgagcatggccttatactttggttcaagaaatacttttacttccttgaacgaagctcttttacgggccactgataatctttgtttctctgcatcagaaagcggattcccctttctgtatgctcgttttgcgccagatgaggaagtcactgcattttctgtctgcgacatctcgcctcctcaatactcaaacagggatcgtttcgcagaggatactacagttttttgaaatcagcgacttgagaattgtgacgaagatctgtgttggcgcacaaatcaacggggattactgtcgtttaatgtgatttaaactgtgaaatagtatggttttcagttattgaaacgccgggagcggggaaaacttgctttttcccgtttccggggttggacaactgagcaacgcgaaggcgtcagctacgatgttccggggactgctgatcagcccctgttgtattcagccattctccggtcatccgaacgccagcaagacgtagcccagcgcgtcggccaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattc